(19) United States
(12) Patent Application Publication
(10) Pub. No.: US 2024/0138976 A1
(43) Pub. Date: May 2, 2024

(54) PROSTHETIC HEART VALVE

(71) Applicant: 4C Medical Technologies, Inc., Maple Grove, MN (US)

(72) Inventors: Jonathan Berndt, Crystal, MN (US); Jeffrey W. Chambers, Maple Grove, MN (US); Jason S. Diedering, Minneapolis, MN (US); Olivia Johnson, Minneapolis, MN (US); Shelby Jo Byron, Maple Grove, MN (US); Saravana B. Kumar, Minnetonka, MN (US); Jaqueline Wendel, Hopkins, MN (US)

(21) Appl. No.: 18/211,504

(22) Filed: Jun. 19, 2023

Related U.S. Application Data
(60) Provisional application No. 63/353,143, filed on Jun. 17, 2022.
(60) Provisional application No. 63/353,147, filed on Jun. 17, 2022.
(60) Provisional application No. 63/353,154, filed on Jun. 17, 2022.
(60) Provisional application No. 63/353,156, filed on Jun. 17, 2022.
(60) Provisional application No. 63/400,879, filed on Aug. 25, 2022.

(51) Int. Cl. A61F 2/24

(52) U.S. Cl. CPC A61F 2/2418; A61F 2002/0081; A61F 2220/0008; A61F 2250/0039

(57) ABSTRACT

Apparatus and methods for a prosthetic heart valve are provided. The apparatus may include a prosthetic heart-valve fitting. The fitting may be seated in a valve annulus of the heart. The fitting may be a fitting that does not include a tissue-penetrating anchor. The fitting may be a fitting that does not include a tissue-penetrating barb. The apparatus may include a brace. The brace may be urged by an atrial tissue of the heart to maintain a position of the fitting in the annulus.

### CROSS-REFERENCE TO RELATED APPLICATIONS

This application is a nonprovisional of U.S. Provisional Applications Nos. 63/353,147 filed Jun. 17, 2022, 63/353,154 filed Jun. 17, 2022, 63/353,143 filed Jun. 17, 2022, 63/353,156 filed Jun. 17, 2022, and 63/400,879 filed Aug. 25, 2022, all of which are hereby incorporated by reference in their entireties.

### BACKGROUND

The human heart includes four chambers and four heart valves that assist in the forward (antegrade) flow of blood through the heart. The chambers include the left atrium, left ventricle, right atrium, and right ventricle. The four heart valves include the mitral valve, the tricuspid valve, the aortic valve, and the pulmonary valve. See generally FIG. 1 for an anterior view of the heart 102.

Mitral valve 106 is located between the left atrium LA and left ventricle LV and helps control normal antegrade blood flow 112 from the left atrium LA to the left ventricle LV by acting as a one-way valve to prevent backflow into the left atrium LA.

Tricuspid valve 104 controls normal antegrade blood flow 112 from the right atrium RA to the right ventricle RV of the heart, preventing blood from flowing backwards from the right ventricle RV into the right atrium RA so that it is instead forced through the pulmonary valve and into the pulmonary arteries for delivery to the lungs. A properly functioning tricuspid valve opens and closes to enable blood flow in one direction.

These valves are all one-way valves, with leaflets that open to allow forward (antegrade) blood flow. The normally functioning valve leaflets close under the pressure exerted by reverse blood to prevent backflow (retrograde) of the blood into the chamber it just flowed out of. For example, tricuspid valve 104 when working properly provides a one-way valve between right atrium RA and right ventricle RV, opening to allow antegrade blood flow 112 from right atrium RA to right ventricle RV and closing to prevent retrograde blood flow 114 from right ventricle RV into right atrium RA. This retrograde flow may include regurgitation. Mitral valve 106 when working properly provides a one-way valve between left atrium LA and left ventricle LV, opening to allow antegrade blood flow 112 from left atrium LA to left ventricle LV and closing to prevent regurgitative, retrograde blood flow 116 from the left ventricle LV into the left atrium LA.

Native heart valves may be, or become, dysfunctional for a variety of reasons and/or conditions including but not limited to disease, trauma, congenital malformations, and aging. These types of conditions may cause the valve structure to fail to close properly resulting in regurgitant retrograde flow of blood from a ventricle to an atrium.

In some cases, the dysfunction may result from mitral valve 106 leaflet prolapse or deficient coaptation. The dysfunction may cause retrograde blood flow 116 into the left atrium LA. The backflow may place a burden on the left ventricle LV with a volume load that may lead to a series of left ventricular LV compensatory adaptations and adjustments, including remodeling of the ventricular chamber size and shape, that vary considerably during the prolonged clinical course of mitral regurgitation.

Tricuspid valve regurgitative, retrograde blood flow 114 may develop when the tricuspid valve 104 leaflets are unable to close or coapt properly, allowing blood to regurgitate from the right ventricle RV back into the right atrium RA.

Regurgitant, retrograde blood flow 114 through the tricuspid valve 104. Such regurgitation may result in shortness of breath, fatigue, heart arrhythmias, and even heart failure.

A native tricuspid valve 104 typically includes 3 native leaflets (anterior, posterior, and septal) that are inserted into the tricuspid annulus and attached through chordae tendineae to the papillary muscles of the right ventricle RV. The tricuspid annulus may be less fibrous than the mitral annulus, and the right coronary artery may surround the parietal attachment of the valve.

Dysfunctional tricuspid valve native leaflets may fail to properly coapt together and may allow upstream regurgitant flow 114 through gaps between the improperly coapted leaflets as shown in FIG. 1.

Dysfunctional tricuspid valves are also often associated with problems related to the left side of the heart, such as mitral valve regurgitation 116.

Native heart valves, therefore, may require functional repair and/or assistance, including a partial or complete replacement. Such intervention may take several forms including techniques such as open-heart surgery and open-heart implantation of a replacement heart valve.

Less invasive methods and devices for replacing a dysfunctional heart valve are known and may involve percutaneous access and catheter-facilitated delivery of the replacement valve. Some solutions may involve a replacement heart valve attached to a structural support such as a stent or other form of wire network designed to expand upon release from a delivery catheter. If a device is not properly positioned in a positioning attempt, it may be recaptured and positionally adjusted. The recapturing process in the case of a fully, or even partially, expanded device may require re-collapsing the device to a point that allows the operator to retract the collapsed device back into a delivery sheath or catheter, adjust the inbound position for the device and then re-expand to the proper position by redeploying the positionally-adjusted device distally out of the delivery sheath or catheter. Collapsing the already expanded device may be difficult because the expanded stent or wire network may be designed to achieve the expanded state which also resists contractive or collapsing forces.

In tricuspid valve replacement, it may be challenging to gain access to the native valve structure. Because the tricuspid annulus is typically large, known prosthetic tricuspid valves may have a large profile requiring large bore delivery catheters. This may limit access route possibilities. Four access routes are generally used for known tricuspid valve replacement devices: (1) trans-jugular, either surgically or percutaneously accessed (may provide a good angle to approach the tricuspid valve, but requires a vein large enough to accommodate the relatively large delivery catheter); (2) femoral access; (may be the safest route due to its relatively large size, but the resulting angle between the inferior vena cava and the tricuspid valve is steep); (3) transatrial approach (requiring an anterior right thoracotomy; may be used to obtain direct management of the access site); and (4) transapical.

Anchoring may be a challenge with tricuspid valve replacement. The tricuspid annulus is typically not calcific and is a dynamic structure in 3 dimensions.

Interference with the heart's electrical conduction system and pacing devices may be another tricuspid valve replacement challenge. The atrioventricular (AV) node lies in the atrioventricular septum and the bundle of His is a direct continuation of the AV node, passing through the right trigone of the central fibrous body to reach the ventricular septum. The close relationship between the tricuspid valve structure and the conduction system may result in interference of the conduction system during tricuspid valve replacement procedures.

The relatively low flow on the right side of the heart and the relatively large size of tricuspid valve prosthetics may result in thrombosis. Thus, antithrombotic, or anticoagulant therapy may be indicated. Because the tricuspid annulus may be saddle-shaped or irregularly shaped, apposition of a replacement prosthetic may be incomplete, resulting in paravalvular leakage or other residual regurgitation.

### BRIEF DESCRIPTION OF THE DRAWINGS

FIG. 1 illustrates a cross-sectional view of the heart showing certain features of the heart and retrograde blood flow resulting from tricuspid and mitral valve regurgitation compared with normal blood flow.

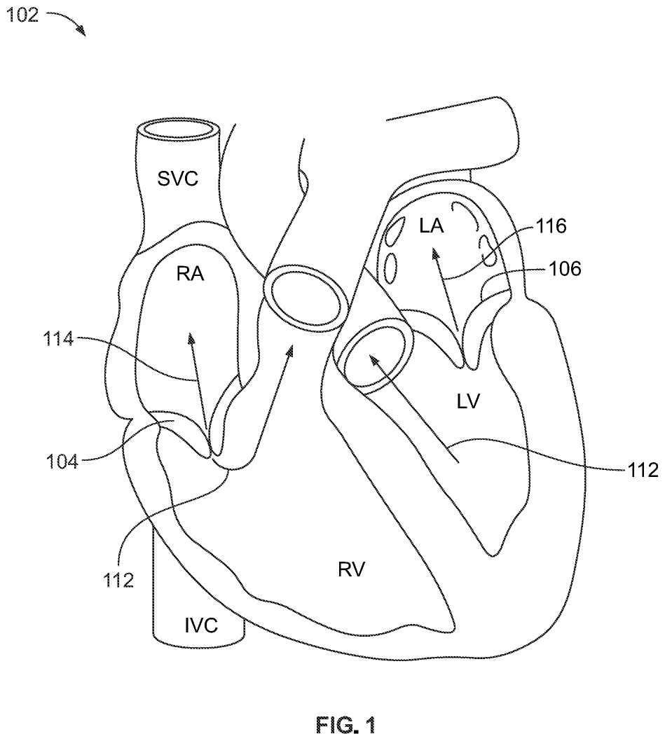

FIG. 2 illustrates schematically apparatus in accordance with the principles of the present invention.

FIG. 3 illustrates schematically apparatus in accordance with the principles of the present invention.

FIG. 4 illustrates schematically apparatus in accordance with the principles of the present invention.

FIG. 5 illustrates schematically apparatus in accordance with the principles of the present invention.

FIG. 6 illustrates schematically apparatus in accordance with the principles of the present invention.

FIG. 7 illustrates schematically apparatus in accordance with the principles of the present invention.

FIG. 8 illustrates schematically apparatus in accordance with the principles of the present invention.

FIG. 9 illustrates schematically apparatus in accordance with the principles of the present invention.

FIG. 10 illustrates schematically apparatus in accordance with the principles of the present invention.

FIG. 11 shows a view analogous to a partial view of FIG. 10 taken along view lines 11-11.

FIG. 12 illustrates schematically apparatus in accordance with the principles of the present invention.

FIG. 13 illustrates schematically apparatus in accordance with the principles of the present invention.

FIG. 14 illustrates schematically apparatus in accordance with the principles of the present invention.

FIG. 15 illustrates schematically apparatus in accordance with the principles of the present invention.

FIG. 16 illustrates schematically apparatus in accordance with the principles of the present invention.

FIG. 17 illustrates schematically apparatus in accordance with the principles of the present invention.

FIG. 18 illustrates schematically apparatus in accordance with the principles of the present invention.

FIG. 19 illustrates schematically apparatus in accordance with the principles of the present invention.

FIG. 20 illustrates schematically apparatus in accordance with the principles of the present invention.

FIG. 21 shows a view of FIG. 20 taken along view lines 21-21.

FIG. 22 illustrates schematically apparatus in accordance with the principles of the present invention.

FIG. 23 illustrates schematically apparatus in accordance with the principles of the present invention.

FIG. 24 illustrates schematically apparatus in accordance with the principles of the present invention.

FIG. 25 illustrates schematically apparatus in accordance with the principles of the present invention.

FIG. 26 illustrates schematically apparatus in accordance with the principles of the present invention.

FIG. 27 illustrates schematically apparatus in accordance with the principles of the present invention.

FIG. 28 illustrates schematically apparatus in accordance with the principles of the present invention.

FIG. 29 illustrates schematically apparatus in accordance with the principles of the present invention.

FIG. 30 shows a view of FIG. 29 taken along view lines 30-30.

FIG. 31 illustrates schematically apparatus in accordance with the principles of the present invention.

FIG. 32 shows a view of FIG. 31 taken along view lines 32-32.

FIG. 32A shows a view of FIG. 31 taken along view lines 32A-32A.

FIG. 33 illustrates schematically apparatus in accordance with the principles of the present invention.

FIG. 34 shows a view of FIG. 33 taken along view lines 34-34.

FIG. 35 illustrates schematically apparatus in accordance with the principles of the present invention.

FIG. 36 shows a view of FIG. 35 taken along view lines 36-36.

FIG. 37 illustrates schematically apparatus in accordance with the principles of the present invention.

FIG. 38 shows a view of FIG. 37 taken along view lines 38-38.

FIG. 38A shows a view of FIG. 37 taken along lines 38A-38A.

FIG. 39 illustrates schematically apparatus in accordance with the principles of the present invention.

FIG. 40 illustrates schematically apparatus in accordance with the principles of the present invention.

FIG. 41 illustrates schematically apparatus in accordance with the principles of the present invention.

FIG. 42 illustrates schematically apparatus in accordance with the principles of the present invention.

FIG. 43 illustrates schematically apparatus in accordance with the principles of the present invention.

FIG. 44 illustrates schematically apparatus in accordance with the principles of the present invention.

FIG. 45 illustrates schematically apparatus in accordance with the principles of the present invention.

### DETAILED DESCRIPTION

Apparatus and methods for treating a heart are provided.

The apparatus may include a prosthetic heart-valve fitting. The fitting may be seated in a valve annulus of the heart. The fitting may be a fitting that does not include a tissue-penetrating anchor. The fitting may be a fitting that does not include a tissue-penetrating barb.

The apparatus may include a brace. The brace may be urged by an atrial tissue of the heart to maintain a position of the fitting in the annulus. The brace may have an outer surface. In operation, some or all the outer surface may be in contact with atrial tissue. An upstream section of the brace with respect to a central axis of the apparatus may contact atrial tissue. A central section of the brace with respect to a central axis of the apparatus may contact atrial tissue. A lower section of the brace with respect to a central axis of the apparatus may contact atrial tissue. The brace may contact any tissue in the atrium. The atrial tissue may contact part of the brace which faces the upstream direction. The atrial tissue may contact part of the brace facing a direction which is radially outward from a central axis of the apparatus. The atrial tissue may contact part of the brace in the upstream direction and part of the brace which is radially outward from a central axis of the apparatus. The atrial tissue may be part of the heart's endocardium. The endocardium may be part of an interior, right lateral wall of a right atrium. The endocardium may be part of an interior, left lateral wall of a left atrium.

The fitting may have an inner wall. The fitting may have an outer wall. The fitting may have an annular surface which connects to the inner wall and the outer wall. The fitting may have an inside diameter. The inside diameter may be the diameter of the fitting at an upstream position along the central axis. The fitting may have an outside diameter. The outside diameter may be the diameter of the fitting at an upstream position relative to the central axis. The brace may include a first end. The brace may include a second end. The first end may be a downstream end of the brace. The second end may be an upstream end of the brace. The first end of the brace may extend from the fitting. The first end of the brace may meet the fitting at the plane where the inside diameter of the fitting is measured. The second end of the brace may contact the atrial tissue.

The brace may include a span. The span may extend between the first end and the second end. The span may be cylindrical. The span may have a width. The width may have a minimum value that, when the apparatus is in an unconstrained state, is a fraction of the fitting's outside diameter that may be quantified as ratio R.

Table 1 lists ranges that may include ratio R.

TABLE 1
Illustrative ranges that may include ratio R.
Range

| Lower limit | Upper limit |
|---|---|
| <0.2 | 0.2 |
| 0.2 | 0.22 |
| 0.22 | 0.24 |
| 0.24 | 0.26 |
| 0.26 | 0.28 |
| 0.28 | 0.3 |
| 0.2 | 0.3 |
| 0.3 | 0.32 |
| 0.32 | 0.34 |
| 0.34 | 0.36 |
| 0.36 | 0.38 |
| 0.38 | 0.4 |
| 0.3 | 0.4 |
| 0.4 | 0.42 |
| 0.42 | 0.44 |
| 0.44 | 0.46 |
| 0.46 | 0.48 |
| 0.48 | 0.5 |
| 0.4 | 0.5 |
| 0.54 | 0.56 |
| 0.56 | 0.58 |
| 0.58 | 0.6 |
| 0.5 | 0.6 |
| 0.6 | 0.62 |
| 0.62 | 0.64 |
| 0.64 | 0.66 |
| 0.66 | 0.68 |
| 0.68 | 0.7 |
| 0.7 | .72 |
| .72 | .74 |
| .74 | .76 |
| .76 | .78 |
| .78 | .8 |
| .8 | .82 |
| .82 | .84 |
| .84 | .86 |
| .86 | .88 |
| .88 | .90 |
| .90 | .92 |
| .92 | .94 |
| .94 | .96 |
| .96 | .98 |
| .98 | >.98 |
| Other suitable lower limits | Other suitable upper limits |

Nickel Titanium alloy or any other suitable material may be included in one or more of the apparatus, the fitting, the brace, the span, and any elements thereof.

A cross section of the span may be circular, elliptical, polyhedral, prismatic, triangular, rectangular, pentagonal, hexagonal, octagonal, or of any other suitable shape.

The span may have a length between the first end and the second end. The location of the width may be on a segment of the span. The width of the span may change over the length of the span. The width may be constant over the segment of the span. The segment may be the width of the span. The segment may be the narrowest width of the span. The segment may be an arbitrary location of the span.

The segment may be a fraction F of the length of the span. The center point may be at a location on the span that is at height H with 0 representing the first end and 1.0 representing the second end.

Table 2 lists illustrative ranges that may include the fraction F.

TABLE 2
Illustrative ranges that may include fraction F.
Range

| Lower limit | Upper limit |
|---|---|
| <0.1 | 0.1 |
| 0.1 | 0.2 |
| 0.2 | 0.3 |
| 0.3 | 0.4 |
| 0.4 | 0.5 |
| 0.5 | 0.6 |
| 0.6 | 0.7 |
| 0.7 | 0.8 |
| 0.8 | 0.9 |
| 0.9 | 1.0 |
| Other suitable lower limits | Other suitable upper limits |

The diameter of the span may be constant from the first end to the second end, namely, the width. The diameter of the span may have a minimum diameter at the width. The width may be on the span at a point away from the first end and second end. The diameter of the span may increase gradually from the width until reaching the first end and second end. When the width is smaller than the diameter of the span at the first end, the width may be a segment that has a constant diameter along the length of the segment before the width expands toward the width of the first end. The segment may have a center point that is in middle of the length of the segment. The center point of the segment may be at a location that corresponds to height H of the span. Table 3 lists illustrative ranges that may include the height H as a ratio of the height of the center point of the segment in relation to the span to the length of the span.

TABLE 3
Illustrative ranges that may include height H
(ratio of center point location to span length).
Range

| Lower limit | Upper limit |
|---|---|
| <0.1 | 0.1 |
| 0.1 | 0.2 |
| 0.2 | 0.3 |
| 0.3 | 0.4 |
| 0.4 | 0.5 |
| 0.5 | 0.6 |
| 0.6 | 0.7 |
| 0.7 | 0.8 |
| 0.8 | 0.9 |
| 0.9 | 1.0 |
| Other suitable lower limits | Other suitable upper limits |

When the heart is in a delivery state and the apparatus is deployed in the heart, the brace may provide clearance along an access path. The access path may extend from an entrance of a chamber of the heart to an exit of the chamber of the heart. The clearance may be a clearance that is not less than that required for passage of an instrument. The delivery state may be a state in which the heart contractions follow the patient's native heart rhythm. The delivery state may include pacing the heart at a selected rate such as a rate in the range 190-200 beats per minute. Table 4 illustrates ranges that may include the instrument diameter.

TABLE 4
Illustrative ranges that may include the instrument diameter.
Range

| Lower limit (Fr) | Upper limit (Fr) |
|---|---|
| <3 | 3 |
| 3 | 4 |
| 4 | 5 |
| 5 | 6 |
| 6 | 7 |
| 7 | 8 |
| 8 | 9 |
| 9 | 10 |
| 10 | 11 |
| 11 | 12 |
| 12 | 13 |
| 13 | 14 |
| 14 | 15 |
| 15 | 16 |
| 16 | 17 |
| 17 | 18 |
| 18 | 19 |
| 19 | 20 |
| 20 | 21 |
| 21 | 22 |
| 22 | 23 |
| 23 | 24 |
| 24 | 25 |
| 25 | 26 |
| 26 | 27 |
| 27 | 28 |
| 28 | 29 |
| 29 | 30 |
| 30 | 31 |
| 32 | 33 |
| 33 | 34 |
| 32 | 33 |
| 33 | 34 |
| 34 | 35 |
| 35 | 36 |
| 36 | 37 |
| 37 | 38 |
| 38 | 39 |
| 39 | 40 |
| 40 | >40 |
| Other suitable lower limits | Other suitable upper limits |

The instrument may include a catheter or any other suitable instrument.

The chamber may be a right atrium, a right ventricle, a left atrium, or a left ventricle.

Chamber entrances and exits may include a superior vena cava, an inferior vena cava, a piercing in a septal wall, a pulmonary artery, a heart valve, a native heart valve, a heart valve annulus, a native heart valve annulus and any other suitable entrance or exit.

Table 5 lists illustrative apparatus elements and anatomy between which clearance may be provided.

TABLE 5
Illustrative apparatus elements and anatomy
between which clearance may be provided.
Illustrative apparatus elements and anatomy

Brace, tissue
Fitting, tissue
Brace, fitting, tissue
Span, fitting, tissue
Arms, span, fitting, tissue
Arms, span, stand, tissue
Arms, fitting, tissue
Arms, stand, tissue
Arms, circumferential stabilizer, span, fitting, tissue
Arms, circumferential stabilizer, span, stand, tissue
Arms, circumferential stabilizer, fitting, tissue
Arms, circumferential stabilizer, stand, tissue
Arch, span, fitting, tissue
Arch, span, stand, tissue
Arch, fitting, tissue
Arch, stand, tissue
Support, span, fitting, tissue
Support, span, stand, tissue
Support, fitting, tissue
Support, stand, tissue
Other suitable elements and anatomy

The apparatus may include a window. The window may be sized to accommodate a hot spot of a node of the cardiac conduction system (CCS). The CCS may also be called the electrical system of the heart. The hot spot may also be called a trigger point. The hot spot may be a part of the CCS such as a node that, when stimulated, initiates a contraction of muscles of the heart. The hot spot may be in the sinoatrial node. The sinoatrial node may be in the right atrium of the heart. The window may, when the apparatus is in operation, face the node. The window may be sized to accommodate the node. The window may be sized to accommodate the node and a margin around the node.

By avoiding contact with an element of the CCS, stimulation of the CCS may be avoided. Avoiding stimulation of the CCS may avoid changing a rhythm of contractions of the heart. The node may be the sinoatrial (SA) node. The node may be the atrioventricular (AV) node. The node may be the bundle of His. The node may be the bundle branches. The node may be the Purkinje fibers.

The apparatus may include a brace. The brace may allow the valve to fit in the right atrium in a way that may avoid contact with a hot spot of the sinoatrial node. The brace may allow the valve to fit in the right atrium in a way that may avoid contact with a hot spot of the sinoatrial node. The brace may allow the valve to fit in the right atrium in a way that may avoid contact with a hot spot of the sinoatrial node and a margin around the sinoatrial node. The brace may allow the valve to fit in the right atrium in a way that may distribute pressure against the atrial tissue in a way that avoids stimulation of the CCS. The brace may allow the valve to fit in the right atrium in a way that distributes pressure against the atrial tissue in a way that may avoid changing a rhythm of contractions of the heart.

The apparatus may include a fitting. The fitting may allow the valve to fit in the right annulus in a way that may avoid contact with a hot spot of the atrioventricular node. The fitting may allow the valve to fit into the tricuspid valve in a way that may avoid contact with a hot spot of the atrioventricular node. The fitting may allow the valve to fit in the right atrium in a way that may avoid contact with a hot spot of the atrioventricular node. The fitting may allow the valve to fit in the right atrium in a way that may avoid contact with a hot spot of the atrioventricular node and a margin around the atrioventricular node. The fitting may allow the valve to fit in the right atrium in a way that may distribute pressure against the atrial tissue in a way that avoids stimulation of the CCS. The fitting may allow the valve to fit in the right atrium in a way that distributes pressure against the atrial tissue in a way that may avoid changing a rhythm of contractions of the heart.

The window may be constructed as a hole in a sheet of material. The window may be constructed as a cell in a cellular matrix. The cell may have a size that is similar to that of surrounding cells. The cell may have a size that is larger than that of surrounding cells. The window may be constructed as ends of surrounding cells terminating at a perimeter around, and defining, the window.

The sheet of material may include woven fabric. The sheet of material may include non-woven fabric. The woven or non-woven fabric may be the same as or similar to the material used for a paravalvular leak mitigation skirt. Some or all of one or more of the brace, span, second end, support, arch, arms, circumferential stabilizer, fitting, and stand may be covered with fabric or tissue. The covering may comprise fabric or tissue covering on an inner and/or an outer surface. The covering may help direct blood flow through the valve and through prosthetic leaflets to an outflow end of the valve into the right ventricle.

One or more of the brace, span, second end, support, arch, arms, circumferential stabilizer, fitting, and stand may include cellular matrix. One or more of the brace, span, second end, support, arch, arms, circumferential stabilizer, fitting, and stand may include cellular matrix may include one or more windows.

Table 6 lists illustrative apparatus elements and anatomy within which or between which a window may be provided to avoid contacting the CCS and a margin around the CCS, the CCS, a node of the CCS, or a part of a node of the CCS.

TABLE 6
Illustrative apparatus elements within which
or between which a window may be provided.
Illustrative apparatus elements

Brace,
Fitting,
Brace, fitting,
Span, fitting,
Arms, span, fitting,
Arms, span, stand,
Arms, fitting,
Arms, stand,
Arms, circumferential stabilizer, span, fitting,
Arms, circumferential stabilizer, span, stand,
Arms, circumferential stabilizer, fitting,
Arms, circumferential stabilizer, stand,
Arch, span, fitting,
Arch, span, stand,
Arch, fitting,
Arch, stand,
Support, span, fitting,
Support, span, stand,
Support, fitting,
Support, stand,
Other suitable elements

The margin may have an area that is a percentage of the area of the node. The margin may have an area that is a percentage of the area of the node. The margin may have an area that is a percentage of the area of a portion of the node that is at or near the surface of the atrial tissue. Table 7 lists illustrative ranges that may include the margin area (MA).

TABLE 7
Illustrative ranges that may include the margin area (MA).
Range

| Lower limit | Upper limit (%) |
|---|---|
| (MA, %) | (MA, %) |
| <1 | 1 |
| 2 | 5 |
| 5 | 10 |
| 10 | 20 |
| 20 | 30 |
| 30 | 40 |
| 40 | 50 |
| 50 | 60 |
| 60 | 70 |
| 70 | 80 |
| 80 | 90 |
| 90 | 100 |
| 100 | >100 |
| Other suitable lower limits | Other suitable upper limits |

A skirt may be a woven material such as a woven fabric. A skirt may be a non-woven material such as a non-woven fabric. The skirt may be used, for example, to prevent or minimize leakage of blood through various parts of a prosthetic value. The skirt may be used, for example, to prevent or minimize leakage of blood between various parts of a prosthetic valve and anatomy which surrounds the valve such as tissue. The skirt may be a paravalvular leak mitigation skirt.

A skirt may enhance any part of the apparatus to reduce leakage. The skirt may cover any side of a component of the apparatus. For example, the skirt may cover the inside of the component. The skirt may cover the outside of the component. The skirt may cover, in whole or in part, one or more sides of the apparatus or an element of the apparatus such as the brace, span, second end, support, arch, arms, circumferential stabilizer, fitting, stand, or other suitable component.

The window may distribute force on the atrial tissue to avoid disturbing a rhythm of the heart. The window may distribute mechanical loading from the node to other areas of the tissue to avoid disturbing the rhythm of the heart.

The types of tissue may include heart tissue, atrial tissue, ventricular tissue, annular tissue, septal tissue, and other tissue of the heart and its surroundings.

The types of annuli may include tricuspid annulus, mitral annulus, and other annuli of the heart and its surroundings.

Native tricuspid leaflets may be pinned against an annular tissue by a fitting. The annular tissue may be the tricuspid annular tissue. The fitting may be a fitting that does not extend in the upstream direction a distance that is sufficient to pin the native leaflets. This may leave the tricuspid native leaflet function intact. The native leaflet function may be supplemented by prosthetic leaflets. As the native leaflets continue to degrade in functionality, the prosthetic leaflets may eventually replace the native leaflet function.

The prosthetic leaflets may function as a one-way valve, allowing flow through the tricuspid annulus in the downstream direction while reducing or preventing regurgitative flow in the upstream direction. At least a portion of the outer surface of the fitting may be covered by a skirt covering. The skirt may be a woven fabric. The skirt may be a non-woven fabric. The fabric may aid in apposition. The fabric may reduce or prevent paravalvular leakage. Some or all of the inner surface of the fitting may be covered by a fabric. The fabric may aid in fluid flow. The fabric may prevent or reduce thrombosis. Fabric may cover at least part of the inner surface of the frame, including, optionally, the transition section of frame, with a fabric covering. Fabric may include covering at least part of the outer surfaces of the frame with a fabric covering. A variation or combination including, optionally, a fabric covering may be employed.

The apparatus may include at least one prosthetic valve leaflet. The apparatus may include 2, 3, 4, 5, 6, 7, 8, 9, 10, or any suitable number of leaflets.

The attachment sections for the prosthetic valve leaflet may include brace, span, fitting, and channel.

Table 8 lists illustrative leaflet material.

TABLE 8
Illustrative leaflet material.
Illustrative leaflet material

Natural tissue
Pericardial sac tissue
Porcine pericardium
Bovine pericardium
Polyurethane
Synthetic polymer
Natural polymer
Other suitable leaflet material

The fitting may have an inside diameter. The width, when the apparatus is in an unconstrained state, may be about equal to the inside diameter. The width may have a minimum value that, when the apparatus is in an unconstrained state, is a fraction of the fitting's inside diameter that may be quantified as ratio G.

Table 9 lists ranges that may include ratio G.

TABLE 9
Illustrative ranges that may include ratio G.
Range

| Lower limit | Upper limit |
|---|---|
| <0.2 | 0.2 |
| 0.2 | 0.25 |
| 0.25 | 0.3 |
| 0.3 | 0.35 |
| 0.35 | 0.4 |
| 0.4 | 0.42 |
| 0.42 | 0.44 |
| 0.44 | 0.46 |
| 0.46 | 0.48 |
| 0.48 | 0.5 |
| 0.4 | 0.5 |
| 0.54 | 0.56 |
| 0.56 | 0.58 |
| 0.58 | 0.6 |
| 0.54 | 0.56 |
| 0.56 | 0.58 |
| 0.58 | 0.6 |
| 0.5 | 0.6 |
| 0.6 | 0.62 |
| 0.62 | 0.64 |
| 0.64 | 0.66 |
| 0.66 | 0.68 |
| 0.68 | 0.7 |
| 0.7 | .72 |
| .72 | .74 |
| .74 | .76 |
| .76 | .78 |
| .78 | .8 |
| .8 | .82 |
| .82 | .84 |
| .84 | .86 |
| .86 | .88 |
| .88 | .90 |
| .90 | .92 |
| .92 | .94 |
| .94 | .96 |
| .96 | .98 |
| .98 | 1.0 |
| 1.0 | 1.05 |
| 1.05 | 1.1 |
| 1.1 | 1.15 |
| 1.15 | >1.15 |
| Other suitable lower limits | Other suitable upper limits |

The fitting may define a central axis. The fitting may include an outer wall. The outer wall may define the central axis. The fitting may include an inner wall. The inner wall may define the central axis. The inner wall may be coaxial with the outer wall. The fitting may include an annular web. The annular web may extend from the inner wall to the outer wall.

The outer wall may maintain a constant radius. The outer wall may taper. The outer wall may taper from a first radius to a second radius. The first radius may be on a downstream side of the fitting. The second radius may be on an upstream side of the fitting. The second radius may be smaller than the first radius such that the taper resists passage through the annulus.

The apparatus may include a support. The support may be part of the brace. The support may define a semispherical surface. The support may include a dome. The support may extend from the second end. The support may extend away from the second end. The support may extend away from the second end in a direction that is initially perpendicular to the span. The support may extend away from the second end in a direction that is initially perpendicular to the span and then curve until it approaches a direction that is parallel with the span. The support may extend in a direction that abuts the atrial tissue on at least one side. The span may have a length. The length may extend in the between the first and the second end. The support may include a lower edge. The lower edge may be spaced apart from the span. The lower edge may extend downstream relative to the second end. The lower edge may be disposed, when the apparatus is in an unconstrained state, opposite a location on the span that is a maximum of fraction I of the length from the second end.

Table 10 lists ranges that may include fraction I.

TABLE 10
Illustrative ranges that may include fraction I.
Range

| Lower limit | Upper limit |
|---|---|
| <0.05 | 0.05 |
| 0.05 | 0.1 |
| 0.1 | 0.2 |
| 0.2 | 0.22 |
| 0.22 | 0.24 |
| 0.24 | 0.26 |
| 0.26 | 0.28 |
| 0.28 | 0.3 |
| 0.2 | 0.3 |
| 0.3 | 0.32 |
| 0.32 | 0.34 |
| 0.34 | 0.36 |
| 0.36 | 0.38 |
| 0.38 | 0.4 |
| 0.3 | 0.4 |
| 0.4 | 0.42 |
| 0.42 | 0.44 |
| 0.44 | 0.46 |
| 0.46 | 0.48 |
| 0.48 | 0.5 |
| 0.4 | 0.5 |
| Other suitable lower limits | Other suitable upper limits |

The apparatus may include a cap. The cap may retain free ends of struts in a cellular matrix. The cap may include a ring that may retain the free ends. A cap may force a curvature in an element. A cap may force an element to be asymmetrical with respect to a central axis of the apparatus. The asymmetry may align with the blood flow more closely through an atrium of the heart such as the right atrium.

The cap may be collapsible. The cap may be expandable. The cap may engage an expanded configuration the upper tissue of the atrial chamber. The cap may be a cap that is not be expandable. The cap may be a cap that is not be collapsible. The cap may be configured to abut atrial tissue.

The apparatus may include an arm. The arm may be part of the brace. The arm may extend from the second end to a free end. The arm may extend away from the second end.

The arm may extend away from the second end in a direction that is initially perpendicular to the span. The arm may extend away from the second end in a direction that is initially perpendicular to the span and then curve until it approaches a direction that is parallel with the span. The arm may extend in a direction that abuts the atrial tissue.

The apparatus may include two arms. Each arm may extend from the second end to a free end. The two arms, when the apparatus is in an unconstrained state, may be disposed at arc length D, center-to-center of the arms, about the central axis, away from each other. Each arm may abut the atrial tissue.

Table 11 lists ranges that may include arc length D.

TABLE 11
Illustrative ranges that may include arc length D.
Range (°)

| Lower limit (°) | Upper limit (°) |
|---|---|
| <5 | 5 |
| 5 | 10 |
| 10 | 15 |
| 15 | 20 |
| 20 | 25 |
| 25 | 30 |
| 30 | 35 |
| 35 | 40 |
| 40 | 45 |
| 45 | 50 |
| 50 | 55 |
| 55 | 60 |
| 60 | 65 |
| 65 | 70 |
| 70 | 75 |
| 75 | 80 |
| 80 | 85 |
| 85 | 90 |
| 90 | 95 |
| 95 | 100 |
| 100 | 105 |
| 105 | 110 |
| 110 | 115 |
| 115 | 120 |
| 120 | 125 |
| 125 | 130 |
| 130 | 135 |
| 135 | 140 |
| 140 | 145 |
| 145 | 150 |
| 150 | 155 |
| 155 | 160 |
| 160 | 165 |
| 165 | 170 |
| 170 | 175 |
| 175 | 180 |
| Other suitable lower limits | Other suitable upper limits |

The apparatus may contain one or more arms. The apparatus may contain 1, 2, 3, 4, 5, 6, 7, 8, 9, 10, or any suitable number of arms. Each arm may extend from the second end to a free end. The arms, when the apparatus is in an unconstrained state, may be disposed, about the central axis, an equal amount of angular degrees distance from each other, such as an arc length D. The arms may be disposed, about the central axis, a non-equal amount of angular degrees distance from each other, such as one or more different arc lengths D.

When the apparatus contains two or more arms, the apparatus may have a circumferential stabilizer that extends circumferentially, relative to the central axis, from one arm to another arm. The circumferential stabilizer may include a crosspiece. The circumferential stabilizer may allow for less thick arms to be used while still avoiding buckling of the apparatus when deployed in the heart. Thickness of the arms may refer to the thickness of the arms in a circumferential direction around the central axis. Thickness of the arms may refer to the thickness of the arms in a radial direction perpendicular to the central axis. The circumferential stabilizer may be disposed, in a direction of the central axis, in a plane that is perpendicular to the central axis. The circumferential stabilizer may be disposed, in a direction of the central axis, in a plane that is not perpendicular to the central axis. A combination of one or more circumferential stabilizers and two or more arms may extend circumferentially for 360°. They may extend circumferentially, in a direction of the central axis, in a plane that is perpendicular to the central axis. When the plane is perpendicular to the central axis, the height of the plane may be equidistance between a free end of an arm and the second end. The height may be a height that is not equidistance between the free end of an arm and the second end.

The apparatus may include an arm. The arm may be part of the brace. The arm may extend from the fitting to a free end. The arm may extend from one position on the fitting to another position on the fitting. The arm may extend away from the fitting in a direction that is parallel to the central axis. The arm may initially extend away from the fitting in a direction that is parallel to the central axis and then curves toward the central axis.

When the apparatus contains a stand, the arm may extend from the stand to a free end. The arm may extend from one position on the stand to another position on the stand. The arm may extend away from the stand in a direction that is parallel to the central axis. The arm may initially extend away from the stand in a direction that is parallel to the central axis and then curves toward the central axis.

The arm may extend in a direction that abuts the atrial tissue.

The apparatus may include two arms. Each arm may be part of the brace. Each arm may extend from the fitting to a free end. The arms may extend each from a separate position on the fitting to another, separate position on the fitting. The arms may extend away from the fitting in a direction that is parallel to the central axis. The arms may initially extend away from the fitting in a direction that is parallel to the central axis and then curves toward the central axis.

When the apparatus contains a stand, the arms may extend from the stand to a free end. The arms may extend each from a separate position on the stand to another position on the stand. The arm may extend away from the stand in a direction that is parallel to the central axis. The arm may initially extend away from the stand in a direction that is parallel to the central axis and then curves toward the central axis.

The two arms, when the apparatus is in an unconstrained state, may be disposed an arc length D, about the central axis, away from each other. See Table 11. Each arm may abut the atrial tissue. The arms may extend in a direction that abuts the atrial tissue.

The apparatus may contain one or more arms. The apparatus may contain 1, 2, 3, 4, 5, 6, 7, 8, 9, 10, or any suitable number of arms. Each arm may extend from the fitting to a free end. The arms may extend from one position on the fitting to another position on the fitting. The arms may extend away from the fitting in a direction that is parallel to the central axis. The arms may initially extend away from the fitting in a direction that is parallel to the central axis and then curves toward the central axis.

When the apparatus contains a stand, each arm may extend from the stand to a free end. The arms may extend from one position on the stand to another position on the stand. The arms may extend away from the stand in a direction that is parallel to the central axis. The arms may initially extend away from the stand in a direction that is parallel to the central axis and then curves toward the central axis.

The arms, when the apparatus is in an unconstrained state, may be disposed, about the central axis, an equal amount of angular degrees distance from each other, such as D. See Table 11. The arms may be disposed, about the central axis, a non-equal amount of angular degrees distance from each other.

The arms may intersect each other. The arms may intersect each other at an apex. The arms may intersect each other at an apex along the central axis.

When the apparatus contains two or more arms, the apparatus may have a circumferential stabilizer. The circumferential stabilizer may extend circumferentially, relative to the central axis, from one arm to another arm. The circumferential stabilizer may be disposed, in a direction of the central axis, in a plane that is perpendicular to the central axis. The circumferential stabilizer may be disposed, in a direction of the central axis, in a plane that is not perpendicular to the central axis. A combination of one or more circumferential stabilizers and two or more arms may extend circumferentially for 360°. They may extend circumferentially, in a direction of the central axis, in a plane that is perpendicular to the central axis. When the plane is perpendicular to the central axis, the height of the plane may be equidistance between a fitting and an apex of one of the arms, for example, the higher apex. The height of the plane may be equidistance between a stand and an apex of one of the arms, for example, the higher apex. The height may be a height that is not equal to the distance between the fitting and the higher apex. The height may be a height that is not equidistance between the stand and the higher apex.

The apparatus may include a support. The support may define an arcuate surface. The support may be part of the brace. The support may extend between a first boundary of the support and a second boundary of the support.

The apparatus may include a span. The apparatus may be an apparatus that does not include a span.

When the apparatus includes a span, the first arcuate boundary may be associated with the second end. The first arcuate boundary may have a part that extends from one side of the second end down to the fitting and another part that extends from another side of the second end down to the fitting on another side. The two parts, when the apparatus is in an unconstrained state, may be disposed arc length D, about the central axis, away from each other on the second end. Valves for arc length D may be found in Table 11. These two parts may extend in the shape of an arc. Together, the two parts and the second end may form the first arcuate boundary. When the apparatus contains a stand, the two parts may extend down to the stand instead of directly to the fitting.

When the apparatus does not include a span, the first arcuate boundary may be defined by an arcuate curve that extends from a first position on the fitting to a second position on the fitting. The two positions, when the apparatus is in an unconstrained state, may be disposed arc length D, about the central axis, away from each other on the fitting. Valves for arc length D may be found in Table 11.

When the apparatus contains a stand, the first arcuate boundary may be defined by an arcuate curve that extends from the first position on the stand to the second position on the stand. The two positions, when the apparatus is in an unconstrained state, may be disposed arc length D, about the central axis, away from each other on the fitting. Valves for arc length D may be found in Table 11.

The second arcuate boundary may be defined by the fitting's outside diameter from one point where the first arcuate boundary contacts the fitting to the second point where the first arcuate boundary contacts the fitting. When the apparatus contains a stand, the second arcuate boundary may be defined by the stand's outside diameter from the first position on the stand where the first arcuate boundary contacts the stand to the second position on the stand where the first arcuate boundary contacts the stand.

The apparatus may define a window. The window may be defined by the first arcuate boundary and an edge. The edge may be a part of the fitting's outside diameter which is not included in the second arcuate boundary. When the apparatus contains a stand, the edge may be a part of the stand's outside diameter which is not included in the second arcuate boundary. The window may provide clearance.

The arcuate support may include a convex surface which may abut atrial tissue.

The apparatus may include an arch. The arch may be part of the brace. The arch may have a highest point referred to as an apex.

The apparatus may include a span. The apparatus may be an apparatus that does not include a span.

The arch may extend from one location on the fitting to another location on the fitting.

When the apparatus contains a stand, the arch may extend from a stand. The arch may extend from one location on the stand to another location on the stand.

The arch may be offset from a central axis defined by the fitting. The arch may contact the fitting at two bases. When the apparatus contains a stand, the arch may contact the stand at two bases. The distance of each base from the other may be measured in circular arc, when the apparatus is in an unconstrained state, about the central axis. The larger circular arc may be called the major arc (MaA). The smaller circular arc may be called the minor arc (MiA). Table 12 lists ranges that may include arc length MaA.

TABLE 12
Illustrative ranges that may include arc length MaA.
Range

| Lower limit (°) | Upper limit (°) |
|---|---|
| 355 | >355 |
| 350 | 355 |
| 340 | 350 |
| 330 | 340 |
| 320 | 330 |
| 310 | 320 |
| 300 | 310 |
| 290 | 300 |
| 280 | 290 |
| 270 | 280 |
| 260 | 270 |
| 250 | 260 |
| 240 | 250 |
| 230 | 240 |
| 220 | 230 |
| 210 | 220 |
| 200 | 210 |
| 190 | 200 |
| 185 | 190 |
| >180 | 185 |
| Other suitable lower limits | Other suitable upper limits |

Table 13 lists ranges that may include arc length MiA.

TABLE 13
Illustrative ranges that may include arc length MiA.
Range

| Lower limit (°) | Upper limit (°) |
|---|---|
| <5 | 5 |
| 5 | 10 |
| 10 | 15 |
| 15 | 20 |
| 20 | 25 |
| 25 | 30 |
| 30 | 35 |
| 35 | 40 |
| 40 | 45 |
| 45 | 50 |
| 50 | 55 |
| 55 | 60 |
| 60 | 65 |
| 65 | 70 |
| 70 | 75 |
| 75 | 80 |
| 80 | 85 |
| 85 | 90 |
| 90 | 95 |
| 95 | 100 |
| 100 | 105 |
| 105 | 110 |
| 110 | 115 |
| 115 | 120 |
| 120 | 125 |
| 125 | 130 |
| 130 | 135 |
| 135 | 140 |
| 140 | 145 |
| 145 | 150 |
| 150 | 155 |
| 155 | 160 |
| 160 | 165 |
| 165 | 170 |
| 170 | 175 |
| 175 | 180 |
| Other suitable lower limits | Other suitable upper limits |

When the apparatus includes a span, the arch may contact the span. The arch may be an arch that does not contact the span.

The arch may include a convex surface which may abut atrial tissue. The apex may abut atrial tissue.

The apparatus may include an ellipsoid. The ellipsoid may be part of the brace. The ellipsoid may extend from the fitting. When the apparatus contains a stand, the ellipsoid may extend from the stand.

The ellipsoid may extend in a direction that abuts the atrial tissue.

The ellipsoid may have a major axis. The ellipsoid may have a first minor axis. The ellipsoid may have a second minor axis.

The major axis may be transverse to the central axis. The minor axes may lie in a plane that includes the central axis. The minor axes may lie in a plane that is parallel to the central axis.

Table 14 lists ranges of ratios of three orthogonal axes.

TABLE 14
Illustrative ranges of axes.
Range, ratios of orthogonal axes

| First minor axis: | | Second minor axis: | |
|---|---|---|---|
| Major axis | | First minor axis | |
| Lower limit | Upper limit | Lower limit | Upper limit |
| <0.2 | 0.2 | <0.2 | 0.2 |
| 0.2 | 0.3 | 0.2 | 0.3 |
| 0.3 | 0.4 | 0.3 | 0.4 |
| 0.4 | 0.5 | 0.4 | 0.5 |
| 0.5 | 0.6 | 0.5 | 0.6 |
| 0.6 | 0.7 | 0.6 | 0.7 |
| 0.7 | 0.8 | 0.7 | 0.8 |
| 0.8 | 0.9 | 0.8 | 0.9 |
| 0.9 | >0.9 | 0.9 | >0.9 |
| Other suitable | Other suitable | Other suitable | Other suitable |
| lower limits | upper limits | lower limits | upper limits |

The ellipsoid may have two ends. The ends may be opposite each other along the major axis. At one end, the ellipsoid may contain a protrusion. At both ends, the ellipsoid may contain protrusions.

Two ends of the ellipsoid on opposite ends of the major axis may be wedged into openings of the inferior vena cava (IVC) and the superior vena cava (SVC). The pressure from being wedged into the openings may apply help keep the fitting within the annulus.

One end of the ellipsoid along the major axis may be wedged into the IVC. One end of the ellipsoid may be wedged into the SVC.

The opening of the IVC may include an inflection point or surface. The opening of the SVC may include an inflection point or surface.

One or both of ends of the ellipsoid may include a nubbin. The nubbin may define an inflection surface. The nubbin inflection surface may be sized to correspond to an inflection surface of the opening. The nubbin may therefore fit into the opening and the inflection points would conform to each other to provide resistance to further penetration through the opening. The ellipsoid may thus be wedged between the IVC and SVC and may stabilize the fitting within the annulus.

The ellipsoid may contain a cap. The cap may be positioned along the central axis. The cap may be positioned in a location that is not along the central axis.

The ellipsoid may contain a window. The window may be located along the central axis. The window may be positioned in a location that is not along the central axis. The window may be located on a side of the ellipsoid that is opposite the fitting. When the apparatus contains a stand, the window may be located on a side of the ellipsoid that is opposite the stand.

The ellipsoid may be wedged into the IVC. The ellipsoid may contain a mesh size that permits a catheter to be passed through the IVC and through the mesh. The catheter may proceed through the window of the ellipsoid into the right atrium. The catheter may proceed through the SVC out of the right atrium.

The ellipsoid may be wedged into the SVC. A catheter may process through the IVC into the right atrium. The catheter may proceed through the window into the ellipsoid. The ellipsoid may contain a mesh size that permits a catheter to be passed through the mesh, through the SVC, and out of the right atrium.

The ellipsoid may be symmetrical. The ellipsoid may be asymmetric.

The ellipsoid-containing prosthetic valve may be deployed into the annulus of the right atrium. The valve may self-expand into the right atrium. The elliptical shape of the valve may help the valve to orient such that the two ends along the major axis orient into the IVC and SVC, providing a stabilizing force to keep the fitting in the tricuspid annulus.

The ellipsoid-containing prosthetic valve with a bulge on one of the two major axis may be deployed into the annulus of the right atrium. The valve may self-expand into the right atrium. The elliptical shape of the valve may help the valve to orient such that the two ends along the major axis orient toward the IVC and SVC, with the end with the bulge going wedging into the IVC, providing a stabilizing force to keep the fitting in the tricuspid annulus.

The apparatus may include a prosthetic heart-valve fitting that: is configured to be seated in a valve annulus of the heart; and includes an anchoring element to maintain a position of the fitting in the annulus; and a brace that is configured to be urged by an annulus tissue of the heart to maintain a position of the fitting in the annulus. The anchoring element may include a paddle, a bar, a flange or any other suitable element that is configured to resist displacement of the fitting.

The fitting and the brace may be configured to provide a passage that: extends from an entrance of a chamber to an exit of the chamber; and has, along the passage, a minimum clearance for transit of an instrument when the apparatus is disposed in the heart in its operating position.

The apparatus may include a window. The window may have a size that accommodates a node of the cardiac conduction system. The window, in operation, may face the node. The window may be large enough to circumscribe the node. The window may be large enough to accommodate the node and a margin around the node.

Accommodation of the node by the window may avoid interference of the apparatus with the function of the node in the cardiac conduction system.

The brace may be configured to distribute force on the atrial tissue to maintain a rhythm of the heart. The distribution of force may avoid interference of the apparatus with the function of the node in the cardiac conduction system.

The annulus tissue may include right atrium annulus tissue. The annulus tissue may include tricuspid valve annulus tissue.

The fitting may include: an outer wall; an inner wall coaxial with the outer wall; and an annular web that extends from the inner wall to the outer wall. The outer wall may taper from a first radius to a second radius smaller than the first radius such that the first radius resists passage through the annulus. The support may include a convex surface configured to abut atrial tissue.

The apparatus may be monolithic. A monolithic apparatus may include one or more of the fitting, brace, channel, span, arm, circumferential stabilizer, stand, support, arch, ellipsoid, and any other suitable apparatus elements. The apparatus may be formed as a monolithic article. The apparatus may be delivered as a monolithic piece.

One or more of the parts of the apparatus may be assembled outside the patient and delivered in an assembled state. One or more of the fitting, brace, channel, span, arm, circumferential stabilizer, stand, support, arch, ellipsoid, leaflet, prosthetic leaflet, and any other suitable apparatus elements, may be assembled outside the patient and delivered in an assembled state. One or more of the parts of the apparatus may be assembled using hooks. One or more of the parts of the apparatus may be assembled using sutures. One or more of the parts of the apparatus may be assembled using clips. One or more of the parts of the apparatus may be assembled using snaps.

One or more of the fitting, brace, channel, span, arm, circumferential stabilizer, stand, support, arch, ellipsoid, cap, leaflet, prosthetic leaflet, and any other suitable apparatus elements, may be delivered in an unassembled state. One or more of the fitting, brace, channel, span, arm, circumferential stabilizer, stand, support, arch, ellipsoid, cap, leaflet, prosthetic leaflet, and any other suitable apparatus elements, may be assembled inside the patient. Deployment of the apparatus may thus be performed in one, two, three or more deliveries. The varying parts of the deployed apparatus may contain complementary tabs that allow for the different parts to be locked together in situ.

One or more of the fitting, brace, channel, span, arm, circumferential stabilizer, stand, support, arch, ellipsoid, cap, and any other suitable apparatus elements may be self-expanding. The apparatus may have elasticity that may provide biasing forces against the atrial tissue. The expansion may provide biasing forces against the annulus. One or more of the fitting, brace, channel, span, arm, circumferential stabilizer, stand, support, arch, ellipsoid, cap, leaflet, prosthetic leaflet, and any other suitable apparatus elements may be delivered to the heart in a collapsed configuration. The collapsed configuration may be delivered to the heart through a catheter. The delivery of the collapsed configuration may be through the IVC. The delivery of the collapsed configuration may be through the SVC. The apparatus may be expanded inside the heart by release from the catheter. One or more of the fitting, brace, channel, span, arm, circumferential stabilizer, stand, support, arch, ellipsoid, cap, and any other suitable elements of the apparatus may include mesh. The mesh may be self-expanding. The mesh may be constructed by laser-cutting a nitinol tube. The mesh may be formed into shapes shown and described herein using a mandrill or other suitable forming tool. The mesh may be heat-treated to set a shape. The mesh may have shape-memory behavior.

The apparatus may transit through a lumen of a delivery tube in a collapsed configuration and delivery to the subject heart chamber. The apparatus may transit through a lumen in a catheter. One or more of the fitting, brace, channel, span, arm, circumferential stabilizer, stand, support, arch, ellipsoid, cap, and any other suitable apparatus elements may be shaped to transit through a lumen of a delivery tube in a collapsed configuration.

One or more of the fitting, brace, channel, span, arm, circumferential stabilizer, stand, support, arch, ellipsoid, cap, and any other suitable apparatus elements may be shaped to fit the shape of an atrium, or part of the atrium. One or more of the fitting, brace, channel, span, arm, circumferential stabilizer, stand, support, arch, ellipsoid, cap, and any other suitable apparatus elements may be shaped to fit the shape of the right atrium, or part of the right atrium.

The prosthetic valve may include a prosthetic tricuspid valve. The prosthetic valve may include a prosthetic mitral valve.

The implantation may include anchoring of the prosthetic valve. The anchoring may be in one or more of the right atrium, the right ventricle, the left atrium, and the left ventricle.

The implantation may mitigate against regurgitation.

Apparatus and methods for treating a heart are provided.

The apparatus may include an implant. The implant may include a component.

The component may include: a fitting, a brace, an outer wall, an inner wall, an annular web, a leaflet, a channel, a span, a first end, a second end, a lower edge, a support, a rim, a stand, a dome, a cage, a first edge, a second edge, an arch, a first base, a second base, an apex, an arm, a first arm, a second arm, a crosspiece, and any other suitable component. A component may include a mesh. A mesh may include a cell. A cell may include struts.

A first component and a second component may be engaged to each other prior to deployment in the heart. A first component and a second component may be engaged to each other after deployment in the heart. The component may include one or more of the features shown. They may be engaged by hooks. They may be engaged by sutures. They may be engaged by clips. They may be engaged by snaps.

The apparatus may include a prosthetic heart-valve fitting. The fitting may be configured to be seated in a native valve annulus of the heart. The annulus may be a tricuspid valve annulus (TVA). The annulus may be a mitral valve annulus (MVA). The apparatus may include a brace. The brace may be configured to be urged by atrial tissue of the heart to maintain a position of the fitting in the annulus.

The fitting may be a fitting that does not include a tissue-penetrating anchor. The fitting may be a fitting that does not include a tissue-penetrating barb.

The fitting may define a central axis. The fitting may include an outer wall. The fitting may include an inner wall. The inner wall may be coaxial with the outer wall. The inner wall and outer wall may further be coaxial with the central axis. The fitting may include an annular web. The annular web may extend from the inner wall to the outer wall. The outer wall may taper from a first radius to a second radius smaller than the first radius. The first radius may resist passage through the annulus. The first radius may be closer to the atrium than the second radius. The second radius may be closer to the ventricle than the first radius. The inner wall may be configured to support a valve leaflet. The inner wall may be configured to be fixed to a channel that supports a valve leaflet. The inner wall may be configured to be connected to the channel after the fitting and the channel are separately delivered to the heart. The fitting and the channel may be delivered together to the heart.

The brace may include a first end. The brace may include a second end. The brace may include a span. The span may extend between the first end and the second end. The brace may be configured to be in contact at the second end with the atrial tissue, such as an atrial endocardium. The brace may be configured to maintain a position of the fitting based on contact between atrial tissue and the second end. The first end may be placed over the fitting. The first end may connect to the fitting. The brace may be configured to maintain a position of the fitting based on contact of the brace with the atrial tissue. The endocardium may be an interior, right lateral wall of a right atrium. The endocardium may be an interior, left lateral wall of a left atrium. The second end may connect to the atrial tissue, such as the endocardium. The span may go between the first end and the second. The span may have a constant diameter along its length. The span may be shaped like an hourglass which has a smaller width in the middle and a large width toward the ends. The span may have a bulge in the middle and have a smaller width at the ends.

The fitting may have an outside diameter.

The span may define a surface that in operation faces radially away from the axis. The span may have a width transverse to the axis, at a location on the span. The span may be coaxial with the central axis.

The apparatus may have an unconstrained state. The unconstrained state may include a state in which the apparatus is not compressed by the body, a delivery device, or other constraining element or condition. The apparatus may have a constrained state. The constrained state may include a state in which the apparatus is disposed in a delivery device. The constrained state may include a state in which the apparatus is deployed in the body.

When the apparatus is in an unconstrained state, a ratio of a width of the span to a width of the outer diameter may be between 0.1 and 3.0. It may be between 0.2 and 2.0. It may be between 0.3 and 1.0. It may be between 0.35 and 0.6.

The location of the width may be one quarter of the span away from the first end. The location of the width may be one half of the span away from the first end. The location of the width may be three quarters of the span away from the first end. Illustrative values for the location may be found in Table 15.

Table 15 lists illustrative ranges that may include locations of a width of the span.

TABLE 15
Illustrative ranges that may include locations of the width of the span.
Range

| Lower limit (% of span | Upper limit (% of span |
|---|---|
| away from first end) | away from first end) |
| 0 | 10 |
| 10 | 20 |
| 20 | 30 |
| 30 | 40 |
| 40 | 50 |
| 0 | 50 |
| 25 | 75 |
| 50 | 60 |
| 60 | 70 |
| 70 | 80 |
| 80 | 90 |
| 90 | 100 |
| 50 | 100 |
| Other suitable lower limits | Other suitable upper limits |

The span may have a length between the first end and the second end. The location of the width may be on a segment of the span. The width of the span may change over the length of the span. The width may be constant over the segment of the span. The segment may be the width of the span. The segment may be the narrowest width of the span. The segment may be an arbitrary location of the span.

Table 16 lists illustrative ranges that may include a length of the segment.

TABLE 16
Illustrative ranges that may include a length of the segment.
Range

| Lower limit (cm) | Upper limit (cm) |
|---|---|
| <1 | 1 |
| 1 | 1.2 |
| 1.2 | 1.4 |
| 1.4 | 1.6 |
| 1.6 | 1.8 |
| 1.8 | 2 |
| 1 | 2 |
| 2 | 2.2 |
| 2.2 | 2.4 |
| 2.4 | 2.6 |
| 2.6 | 2.8 |
| 2.8 | 3 |
| 2 | 3 |
| 3 | 3.2 |
| 3.2 | 3.4 |
| 3.4 | 3.6 |
| 3.6 | 3.8 |
| 3.8 | 4 |
| 3 | 4 |
| 4 | 4.2 |
| 4.2 | 4.4 |
| 4.4 | 4.6 |
| 4.6 | 4.8 |
| 4.8 | 5 |
| 4 | 5 |
| 5 | 5.2 |
| 5.2 | 5.4 |
| 5.4 | 5.6 |
| 5.6 | 5.8 |
| 5.8 | 6 |
| 5 | 6 |
| 6 | >6 |
| Other suitable lower limits | Other suitable upper limits |

The brace may include a support extending from the second end. The support may include a convex surface configured to abut the atrial tissue. The convex surface may be configured to conform with the atrial tissue when the apparatus is deployed in the atrium. The span may have a length between the first end and the second end. The support may have a lower edge that is spaced apart from the span. The lower edge may be disposed radially outward from the span. The support may have a lower edge that is disposed, when the apparatus is in an unconstrained state, opposite a location on the span that is no more than one quarter of the length from the second end. The location may be a location that is not more than one half of the length from the second end. The location may be a location that is not more than three quarters from the second end.

The support may have an elliptical shape. The support may have an elliptical shape with a protrusion. The support may have a rectangular shape. The support may have a rectangular shape with a protrusion. The support may have an oblong oval shape with a window. The window may be configured to be opposite the annulus.

The support may define a dome. The dome may include a convex surface configured to abut the atrial tissue. The convex surface may be configured to conform with the atrial tissue when the apparatus is deployed in the atrium. The support may define an arm. The arm may include a convex surface configured to abut the atrial tissue. The convex surface may be configured to conform with the atrial tissue when the apparatus is deployed in the atrium. The support may define a first arm and a second arm. The crosspiece may extend circumferentially, relative to the axis, from the first arm to the second arm. The support may define three arms. The crosspiece may extend circumferentially, relative to the axis, from the first arm to the second arm to the third arm. The support may define four arms. The brace may include a crosspiece. The crosspiece may extend circumferentially, relative to the axis, from the first arm to the second arm to the third arm to the fourth arm. The support may include any suitable number of arms. The support may include any suitable number of crosspieces.

The brace may include a support extending from the second end. The support may include a convex surface configured to abut the atrial tissue. The convex surface may include a configuration that conforms with the atrial tissue. The support may have a lower edge that is spaced apart from the span. The lower edge may be disposed radially outward from the span. Table 17 illustrates ranges that may include the percentage of the distance of the lower edge to the second end to the total length of the span.

TABLE 17
Illustrative ranges that may include the percentage of the distance
of the lower edge to the second end to the total length of the span.
Range

| Lower limit (%) | Upper limit (%) |
|---|---|
| <5 | 5 |
| 5 | 10 |
| 10 | 15 |
| 15 | 20 |
| 20 | 25 |
| 25 | 30 |
| 30 | 35 |
| 35 | 40 |
| 45 | 50 |
| 50 | 55 |
| 55 | 60 |
| 60 | 65 |
| 65 | 70 |
| 70 | 75 |
| 75 | >75 |
| Other suitable lower limits | Other suitable upper limits |

The fitting may define a rim. The support may extend to the rim. The support may be fixed to the rim. The support may be fixed to the rim before deployment in the heart. The support may be fixed to the rim after deployment in the heart.

The apparatus may include a stand. The stand may extend from the rim. The support may extend to the stand. The support may be fixed to the stand. The support and the stand may together define a cage. The support and the fitting may together define a cage.

The cage may form a shape that fits part of the shape of the atrium. The cage may form a shape that fits the entire shape of the atrium. The cage may define a sphere. The cage may define a spheroid. The cage may define an ellipsoid. The cage may define a hull.

The components may include a mesh of cells. The components may be arranged to define openings. The opening may be bounded by the components. A cell may be defined by the struts. The struts may define a void. The struts may circumscribe the void. The struts may include a biocompatible material. The struts may include nickel titanium. Nickel titanium may be known as Nitinol. The apparatus may include a cap. The cap may include a hub. The hub may hold free ends of the struts. The apparatus may be an apparatus that does not include a cap.

A void may have a size. The size may permit passage of an instrument through a mesh. The size may prohibit passage of the instrument through the mesh.

The instrument may include a catheter. The catheter may be used to deliver an apparatus. The delivery may be to the heart. The instrument may include a delivery device. The instrument may include an electrophysiological therapy device. The instrument may include a pacer.

A window may have a size. The size may permit passage of an instrument through a mesh. The size may prohibit passage of the instrument through the mesh.

The instrument may include a catheter. The instrument may include a delivery device. The instrument may include an electrophysiological therapy device. Instruments include a catheter and a pacer. Another example are instruments used to do a procedure on other parts of the heart such as the septum. The instruments may enter the atrium from, or exit to, the inferior vena cava (IVC). The instruments may enter the atrium from, or exit to, the superior vena cava (SVC). The instrument may enter, or exit from, the interior of the apparatus. The instruments may come from any location that allows for access to the heart. The cell density must be sufficiently high to avoid a particular piece from buckling on itself. The instrument may have a diameter. The instrument may have a diameter that is sufficiently small to pass through the support. The support may be configured such that an instrument may pass around it. The window may be configured such that the instrument may pass through it.

Table 18 illustrates ranges that may include the instrument diameter.

TABLE 18
Illustrative ranges that may include the instrument diameter.
Range

| Lower limit (mm) | Upper limit (mm) |
|---|---|
| <1 | 1 |
| 1 | 1.5 |
| 1.5 | 2 |
| 2 | 2.5 |
| 2.5 | 3 |
| 1 | 3 |
| 3 | 3.5 |
| 3.5 | 4 |
| 4 | 4.5 |
| 4.5 | 5 |
| 3 | 5 |
| 5 | 5.5 |
| 5.5 | 6 |
| 6 | 6.5 |
| 6.5 | 7 |
| 5 | 7 |
| 7 | 7.5 |
| 7.5 | 8 |
| 8 | 8.5 |
| 8.5 | 9 |
| 7 | 9 |
| 9 | 9.5 |
| 9.5 | 10 |
| 10 | 10.5 |
| 10.5 | 11 |
| 9 | 11 |
| 11 | 11.5 |
| 11.5 | 12 |
| 12 | 12.5 |
| 12.5 | 13 |
| 11 | 13 |
| 13 | 13.5 |
| 13.5 | 14 |
| 14 | 14.5 |
| 14.5 | 15 |
| 13 | 15 |
| 15 | 15.5 |
| 15.5 | 16 |
| 16 | >16 |
| Other suitable lower limits | Other suitable upper limits |

The fitting may define a rim. The support may contact the rim.

Different components may have different cell densities. Higher cell densities may provide higher mechanical strength. Lower cell densities may provide larger voids for the passage of instruments.

One or more of the fitting, the brace, the span, the support, the rim, the stand, the arm, the crosspiece, and any other suitable component may include mesh.

Table 19 illustrates ranges of cell sizes.

TABLE 19
Illustrative ranges of cell sizes.
Range

| Lower limit (cm) | Upper limit (cm) |
|---|---|
| <1 | 1.0 |
| 1.0 | 1.1 |
| 1.1 | 1.2 |
| 1.2 | 1.3 |
| 1.3 | 1.4 |
| 1.4 | 1.5 |
| 1.0 | 1.5 |
| 1.5 | 1.6 |
| 1.6 | 1.7 |
| 1.7 | 1.8 |
| 1.8 | 1.9 |
| 1.9 | 2.0 |
| 1.5 | 2.0 |
| 2.0 | 2.1 |
| 2.1 | 2.2 |
| 2.2 | 2.3 |
| 2.3 | 2.4 |
| 2.4 | 2.5 |
| 2.0 | 2.5 |
| 2.5 | 2.6 |
| 2.6 | 2.7 |
| 2.7 | 2.8 |
| 2.8 | 2.9 |
| 2.9 | 3.0 |
| 2.5 | 3.0 |
| 3.0 | 3.1 |
| 3.1 | 3.2 |
| 3.2 | 3.3 |
| 3.3 | 3.4 |
| 3.4 | 3.5 |
| 3.0 | 3.5 |
| 3.5 | >3.5 |
| Other suitable lower limits | Other suitable upper limits |

The brace may define a window. The support may include a first edge. The stand may include a second edge. The first edge and second edge together may define the window.

The first edge may lie in a first plane. The first plane may be parallel to the axis. The stand may define a stand diameter.

Table 20 lists illustrative ranges that may include the offset.

TABLE 20
Illustrative ranges that may include the
offset, as a fraction of the stand diameter.
Range

| Lower limit (fraction) | Upper limit (fraction) |
|---|---|
| 0 | 0.1 |
| 0.1 | 0.2 |
| 0.2 | 0.3 |
| 0.3 | 0.4 |
| 0.4 | 0.5 |
| 0.5 | 0.6 |
| 0.6 | 0.7 |
| 0.7 | 0.8 |
| 0.9 | 1.0 |
| Other suitable lower limits | Other suitable upper limits |

The first plane may be offset from the axis by no less than 0.1× the stand diameter, by not less than 0.2× the stand diameter, by not less than 0.3× the stand diameter, by not less than 0.4× the stand diameter, by not less than 0.5× the stand diameter, by not less than 0.6× the stand diameter, by not less than 0.7× the stand diameter, by not less than 0.8× the stand diameter, by not less than 0.9× the stand diameter, or by not less than 1× the stand diameter. The second edge may lie in a second plane. The second edge may be perpendicular to the axis.

The second edge may lie in a plane that is perpendicular to the axis.

The brace may define an arch. The arch may have a first base. The first base may be supported by the fitting. The arch may have a second base. The second base may be supported by the fitting. The arch may have an apex. The axis and the apex may be an axis and apex that do not intersect with each other. The axis and the apex may intersect with each other. The first base and the second base may be spaced apart from each other along the fitting.

The first base and the second base may be spaced apart from each other along the fitting by an arc length about the central axis, as measured center-to-center on the bases. Table 21 illustrates ranges that may include an arc length between the bases.

TABLE 21
Illustrative ranges that may include an arc length between the bases.
Range (°)

| Lower limit | Upper limit |
|---|---|
| <15 | 15 |
| 15 | 30 |
| 30 | 45 |
| 45 | 60 |
| 60 | 75 |
| 75 | 90 |
| 90 | 105 |
| 105 | 120 |
| 120 | 135 |
| 135 | 150 |
| 150 | 165 |
| 165 | 180 |
| Other suitable arc lengths | Other suitable arc lengths |

For the sake of illustration, meshes of the components shown in the drawings may illustrated as solid surfaces with outlines.

The fitting may be a fitting that does not include a tissue-penetrating anchor. The fitting may be a fitting that does not include a tissue-penetrating barb. The fitting and the brace may be configured to be engaged to each other prior to deployment in the heart. The fitting and the brace may be configured to be engaged to each other after deployment in the heart.

The inner wall may be configured to support a valve leaflet. The inner wall may be configured to be fixed to a channel that includes a valve leaflet.

The inner wall may be configured to be connected to the channel after the fitting and the channel are separately delivered to the heart.

The brace may be configured to maintain the position based on contact with a left wall of an atrium of the heart.

The fitting may have an outside diameter. The span may define a surface that in operation faces radially away from the axis. The span may have a width transverse to the axis, at a location on the span; and, when the apparatus is in an unconstrained state: the width may have a predetermined ratio to the outside diameter.

The location may be defined as being one quarter of the span away from the first end. The location may be defined as being one half of the span away from the first end. The location may be defined as being three quarters of the span away from the first end.

The span may have a length between the first end and the second end. The location may be defined as being a segment of the span that: is centered at a midpoint of the span; and the location extends over a quarter of the length. The location may extend over a half of the length. The location may extend over three quarters of the length.

The support may include a convex surface that is configured to abut atrial tissue.

The span may have a length between the first and the second end; the support may include a lower edge that is: spaced apart from the span; and is disposed, when the apparatus is in an unconstrained state, opposite a location on the span that is no more than a predetermined height along the span.

The support may define a dome. The support may define an arm. The fitting may define a rim; and the support may extend to and be fixed to the rim.

The fitting may defines a rim. The stand may extend from the rim; and the support may extend to and be fixed to the stand.

The arm may be a first arm. The support may include a second arm and a crosspiece. The crosspiece may extend circumferentially, relative to the axis, from the first arm to the second arm.

The crosspiece may be disposed, in a direction of the central axis, between the first end and the second end.

The support and the stand together may define a cage. The support may include a first edge. The stand may include a second edge. The first and second edges together may define an opening of the cage.

The first edge may lie in a first plane that is parallel to the axis.

The stand may define a stand diameter. The first plane may be offset from the axis by no less than a predetermined fraction of the stand diameter.

The second edge may lie in a second plane that is perpendicular to the axis.

The second plane may be offset from a midpoint of the length by no less than a predetermined fraction of the length.

The second edge may lie in a plane that is perpendicular to the axis.

The brace defines an arch having: a first base supported by the fitting; a second base supported by the fitting; and an apex. The central axis may be an axis that does not intersect the apex. The first base and the second base may be spaced apart from each other along the fitting.

Illustrative embodiments of apparatus and methods in accordance with the principles of the disclosure will now be described with reference to the accompanying drawings, which form a part hereof. It is to be understood that other embodiments may be utilized and that structural, functional and procedural modifications, additions or omissions may be made, and features of illustrative embodiments, whether apparatus or method, may be combined, without departing from the scope and spirit of the disclosure.

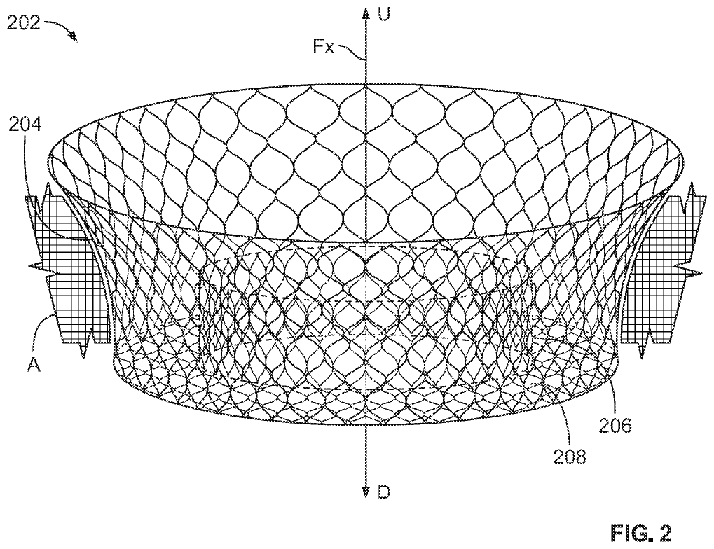

FIG. 2 shows illustrative prosthetic valve fitting 202 in valve annulus A. Fitting 202 may include an inner wall 206. Fitting 202 may include an annular web 208.

Valve 202 may have one or more features in common with one or more of the valves shown or described herein.

Fitting 202 may define a central axis Fx. Axis Fx may define an upstream direction (“U”). Axis Fx may define a downstream direction (“D”). The outer wall 204 of fitting 202 may define an axis that is parallel to axis Fx. The inner wall 206 of fitting 202 may define an axis that is parallel to axis Fx. The axes of fitting 202 and outer wall 204 may be axis Fx. The axes of fitting 202 and inner wall 206 may be axis Fx.

Fitting 202 may include a mesh. A mesh may include a cell. A cell may include struts.

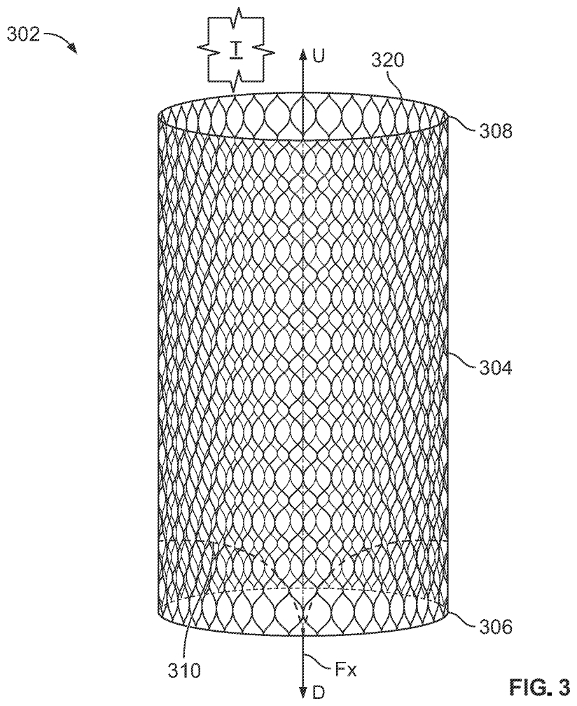

FIG. 3 shows illustrative prosthetic valve brace 302. Brace 302 may include span 304. Brace 302 may include a first end 306. Brace 302 may include a second end 308. Brace 302 may include leaflets 310. Leaflets 310 may be prosthetic leaflets. Span 304 may define an axis parallel to axis Fx. The axis of span 304 may be axis Fx.

Leaflets may be made from various materials. See Table 8.

Brace 302 may include a mesh. A mesh may include a cell. A cell may include struts.

Brace 302 may have an outer surface 320. In operation, some or all the outer surface 320 may be in contact with atrial tissue.

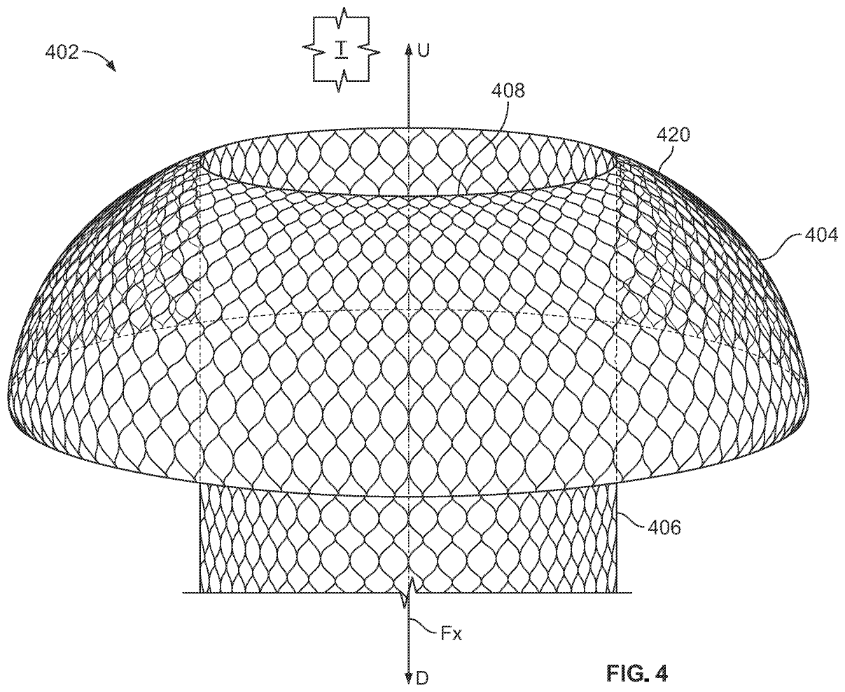

FIG. 4 shows illustrative prosthetic valve upper brace 402. Brace 402 may include support 404. Brace 402 may include span 406. Brace 402 may include a second end 408 in the U direction of the span. Support 404 may begin at second end 408. Support may initially extend away from the second end in a direction that is a direction that is not parallel with axis Fx.

Support 404 may include a mesh. Span 406 may include a mesh. A mesh may include a cell. A cell may include struts.

Brace 402 may have an outer surface 420. In operation, some or all the outer surface 420 may be in contact with atrial tissue.

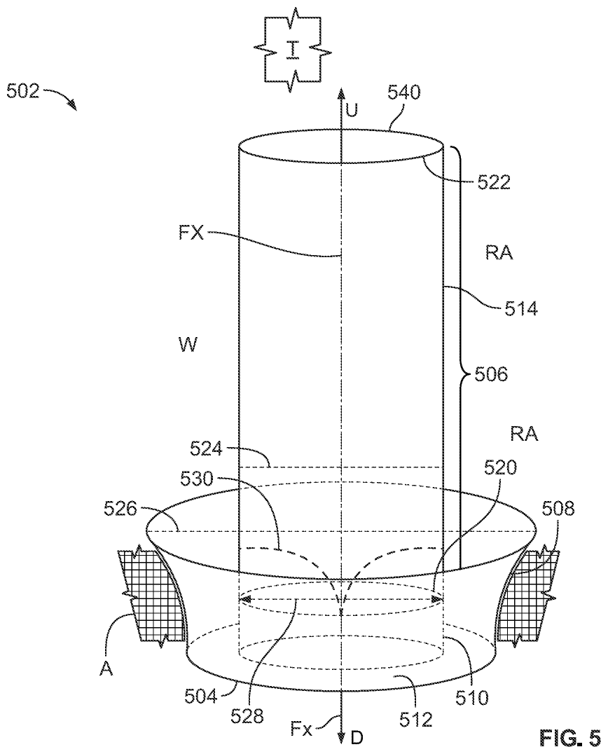

FIG. 5 shows illustrative prosthetic valve 502 in valve annulus A. Valve 502 may include one or more of the features that are shown or described in connection with FIGS. 2-4 and may have one or more features in common with one or more of the other valves shown or described herein. Valve 502 may include fitting 504. Valve 502 may include brace 506.

Fitting 504 may include an inner wall 510. Fitting 504 may include an annular web 512. Fitting 504 may include an outer wall 508. Fitting 504 may have an outside diameter 526. Fitting 504 may have an inside diameter 528.

Brace 506 may include span 514. Brace 506 may have first end 520. Brace 506 may have second end 522. Brace 506 may contact fitting 504 at first end 520. First end 520 may be connected to fitting 504.

Brace 506 may be configured to abut tissue T of right atrium RA at second end 522. Span 514 may in operation bear a compressive force to press fitting 504 into annulus A and brace 506 against tissue T.

Span 514 may have a width 524. Width 524 may be the width of span 514 throughout the length of span 514. Width 524 may equal to inside diameter 528. Width 524 may be smaller than outside diameter 526. Width 524 may be smaller than inside diameter 528. Width 524 may be a ratio R of outside diameter 526. See Table 1. Width 524 may be a ratio G of inside diameter 528. See Table 9.

Span 514 may include one or more prosthetic leaflets 530.

Brace 506 may have one or more elements that transmit force from second end 522.

Brace 506 may have an outer surface 540. In operation, some or all the outer surface 540 may be in contact with atrial tissue.

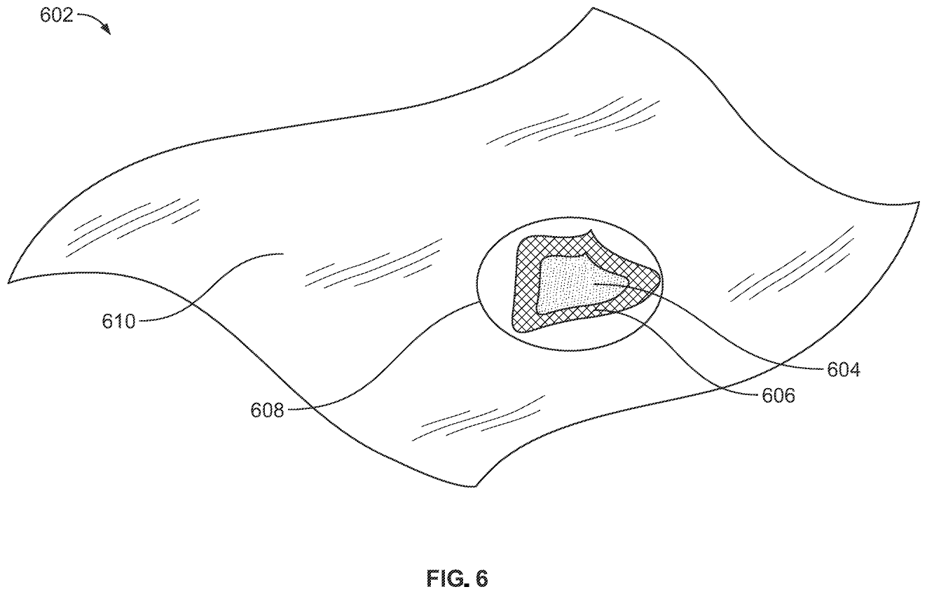

FIG. 6 shows a stretch of an illustrative prosthetic valve 602. Stretch 610 may include a fabric. Stretch 610 may include a woven substance. Stretch 610 may include a non-woven substance. Stretch 610 may be part of a skirt. Stretch 610 may cover a side of a component of valve 602. Stretch 610 may intervene between the component and the heart. For example, stretch 610 may intervene between a fitting and a valve annulus. The skirt may reduce or prevent regurgitation of blood.

Stretch 610 may contact atrial tissue. Stretch 610 may define window 608. Window 608 may be a cutout of stretch 610. Stretch 610 may avoid contacting the atrial tissue at window 608. The atrial tissue may contain part of the cardiac conduction system (CCS) at the place where window 608 is located. For example, window 608 may encompass a hot spot of node 604 of the CCS. Window 608 may encompass node 604 of the CCS. Window 608 may encompass node 604 and margin 606 around node 604 of the CCS.

Window 608 may prevent stretch 610 from contacting a hot spot of node 604. Window 608 may prevent stretch 610 from contacting node 604. Window 608 may prevent stretch 610 from contacting node 604 and a margin 606 around a node. By avoiding contact, stimulation of the CCS may be avoided. Avoiding stimulation of the CCS may avoid changing a rhythm of contractions of the heart. The node may be the sinoatrial (SA) node. The node may be the atrioventricular (AV) node. The node may be the bundle of His. The node may be the bundle branches. The node may be the Purkinje fibers.

Various areas of margin may be provided by a window in addition to the window avoiding the CCS itself. See Table 7.

See Table 6 for illustrative apparatus elements and anatomy which may also provide for a window for avoiding the CCS.

Window 608 may provide clearance for a device to pass through such as a catheter. The device may pass through from an entrance of the atrium to an exit of the atrium. The device may pass through valve 602 at window 608 as it transits through the atrium. The device may be a catheter.

See Table 5 for illustrative apparatus elements and anatomy which may also provide for a window for clearance.

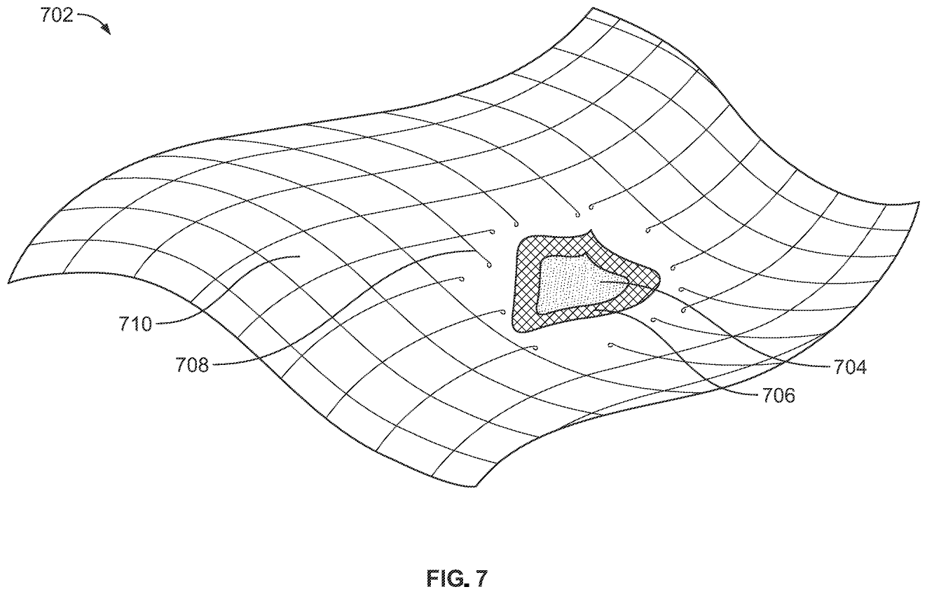

FIG. 7 shows a stretch of an illustrative prosthetic valve 702. Stretch 710 may be a mesh. The mesh may include cells. The cells may include struts. The mesh may have cells which have struts that end in a certain region of stretch 710, thereby defining window 708. Strut ends may be designed to avoid applying point source pressure to the atrial tissue.

Window 708 may prevent stretch 710 from contacting a hot spot of node 704. Window 708 may prevent stretch 710 from contacting node 704. Window 708 may prevent stretch 710 from contacting node 704 and a margin 706 around a node.

Window 708 may provide clearance for a device to pass through such as a catheter. The device may pass through from an entrance of the atrium to an exit of the atrium. The device may pass through valve 702 at window 708 as it transits through the atrium.

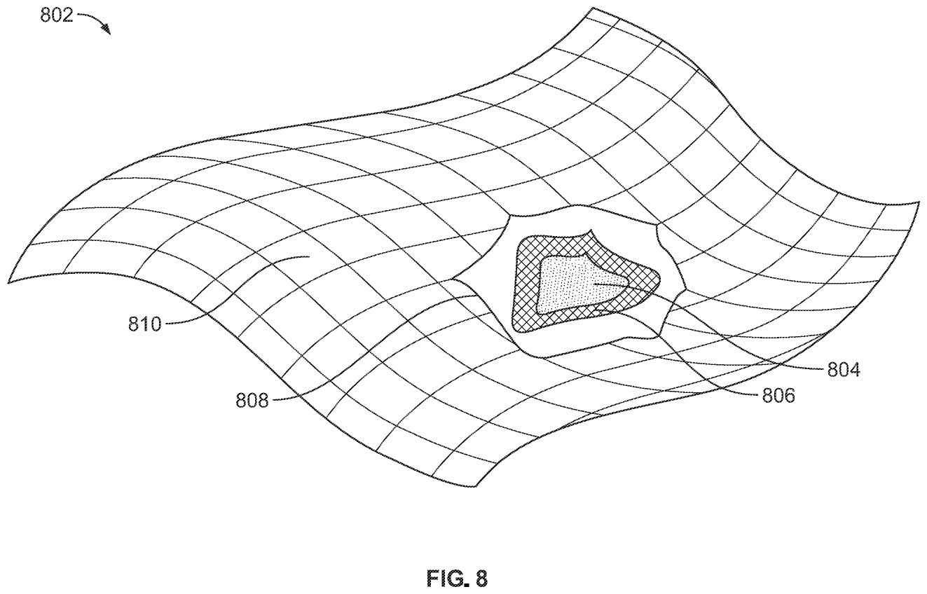

FIG. 8 shows a stretch of an illustrative prosthetic valve 802. Stretch 810 may be a mesh. The mesh may include cells. The cells may include struts. The mesh may have cells of a certain size or sizes which are too small to define a window which may prevent stretch 810 from contacting a hot spot of node 804. The mesh may have cells of a certain size or sizes which are too small to define a window which provides a clearance through stretch 810 of the device. The mesh may contain a larger cell which defines window 808. Window 808 may be sufficiently large to allow for avoiding contact with a hot spot of node 804. Window 808 may be sufficiently large to allow for avoiding contact with node 804. Window 808 may be sufficiently large to allow for avoiding contact with node 804 and margin 806. Window 808 may be sufficiently large to provide clearance for a device to pass through valve 802 such as a catheter.

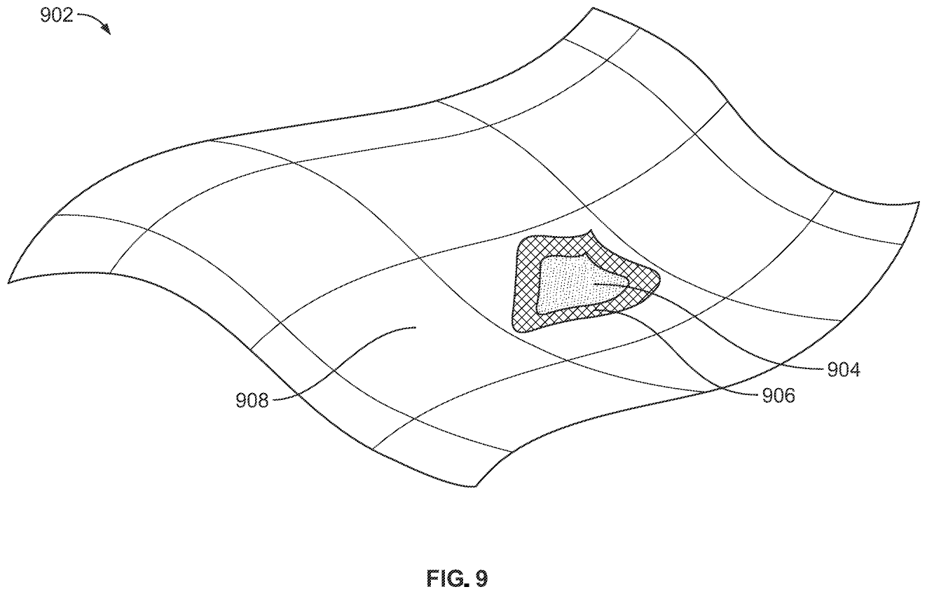

FIG. 9 shows a stretch of an illustrative prosthetic valve 902. Stretch 908 may be a mesh. The mesh may include cells. The cells may include struts. The mesh may have cells of a size or sizes which are sufficiently large to define a window which may prevent stretch 908 from contacting a hot spot of node 904. The mesh may have cells of a size or sizes which are sufficiently large to define a window which may prevent stretch 908 from contacting node 904. The mesh may have cells of a size or sizes which are sufficiently large to define a window which may prevent stretch 908 from contacting node 904 and margin 906. The mesh may be sufficiently large to provide clearance for a device to pass through stretch 908 such as a catheter.

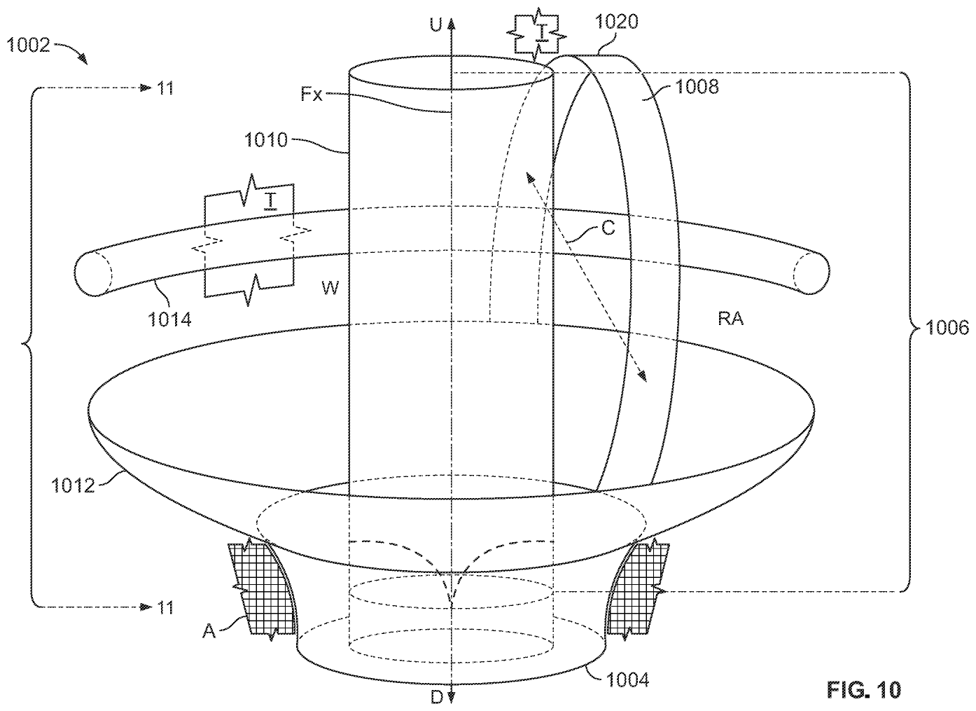

FIG. 10 shows illustrative valve 1002 in valve annulus A. Valve 1002 may include one or more of the features that are shown or described in connection with FIGS. 2-9 and may have one or more features in common with one or more of the other valves shown or described herein. Valve 1002 may include fitting 1004. Valve may include brace 1006. Brace may include stand 1012. Brace may include span 1010. Brace may include arch 1008.

Valve 1002 may illustrate how a device passes through clearance C in valve 1002 to transit from one part of the atrium to another part of the atrium. A catheter 1014 may transit from an entrance into the right atrium such as the IVC or SVC to an exit from the right atrium such as the SVC or IVC. The catheter 1014 may transit from the IVC to the right atrium. When encountering valve 1002, catheter 1014 may pass between span 1010 and the atrial tissue. Catheter 1014 may then continue through arch 1008, which may have clearance C through a window large enough for catheter 1014 to pass through. Catheter 1014 may have a French (Fr) size as found in Table 4.

Brace 1006 may have an outer surface 1020. In operation, some or all the outer surface 1020 may be in contact with atrial tissue.

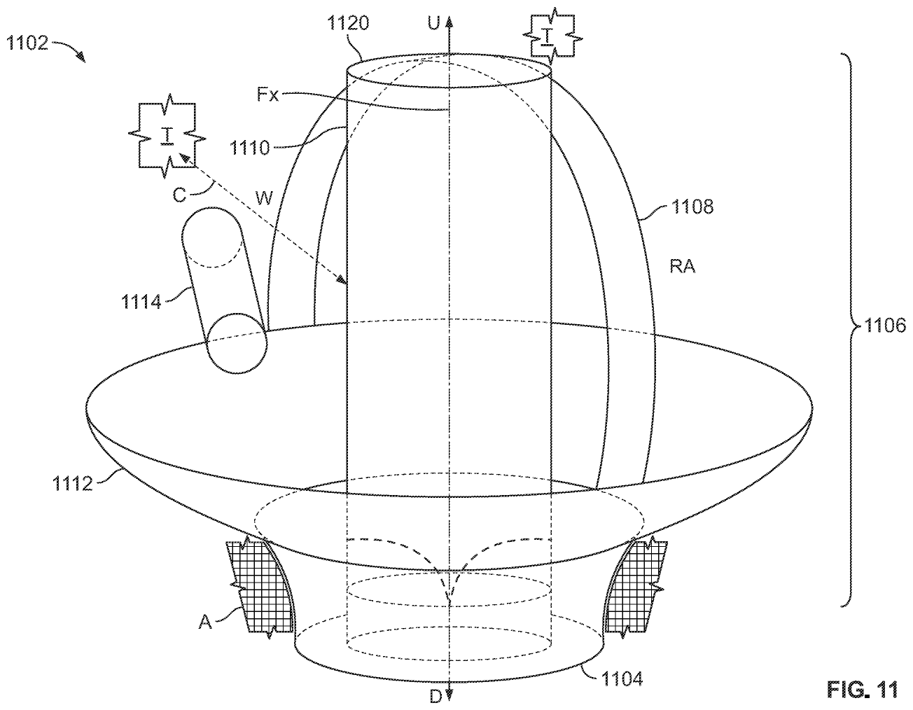

FIG. 11 is a view of valve 1102 in valve annulus A such as that taken along lines 11-11 (shown in FIG. 10). Valve 1102 may include one or more of the features that are shown or described in connection with FIGS. 2-10 and may have one or more features in common with one or more of the other valves shown or described herein. Valve 1102 may include fitting 1104. Valve may include brace 1106. Brace may include stand 1112. Brace may include span 1110. Brace may include arch 1108.

Catheter 1114 may pass by valve 1102 on the side span 1110 and arch 1108.

However, in FIG. 10, catheter 1014 may pass by span 1010 but pass through arch 1008.

When encountering valve 1102, catheter 1114 may pass between span 1010 and the atrial tissue T through clearance C.

Brace 1106 may have an outer surface 1120. In operation, some or all the outer surface 1120 may be in contact with atrial tissue.

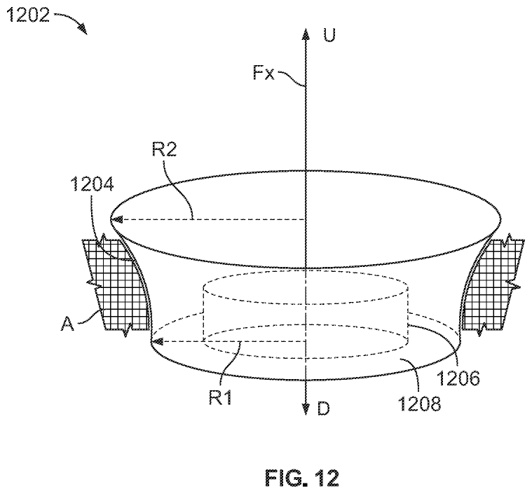

FIG. 12 shows illustrative prosthetic valve fitting 1202 in valve annulus A. Fitting 1202 may include one or more of the features that are shown or described in connection with FIGS. 2-11 and may have one or more features in common with one or more of the other valves shown or described herein. Fitting 1202 may include an outer wall 1204. Fitting 1202 may include an inner wall 1206. Fitting 1202 may include an annular web 1208.

Fitting 1202 may define radius R1. Radius R1 may extend from axis Fx to outer wall 1204. Radius R1 may be a radius of the fitting at downstream end D of fitting 1202. Radius R1 may be a minimum radius of fitting 1202 of any position along axis Fx.

Radius R2 may extends from axis Fx to outer wall 1204. Radius R2 may be a radius of the fitting at upstream direction U of fitting 1202. Radius R2 may be a maximum radius of fitting 1202 at any position along axis Fx.

Radius R1 may be smaller than radius R2. Fitting 1202 may be tapered from radius R2 to radius R1. The taper may help fitting 1202 seat into annulus A. The taper may help fitting 1202 resist passage through annulus A. A ratio of R1 to R2 may be 0.9.

Annular web 1208 may be located at downstream direction D of fitting 1202. Radius R1 may be a radius of annular web 1208. Annular web 1208 may be located at any point along axis Fx.

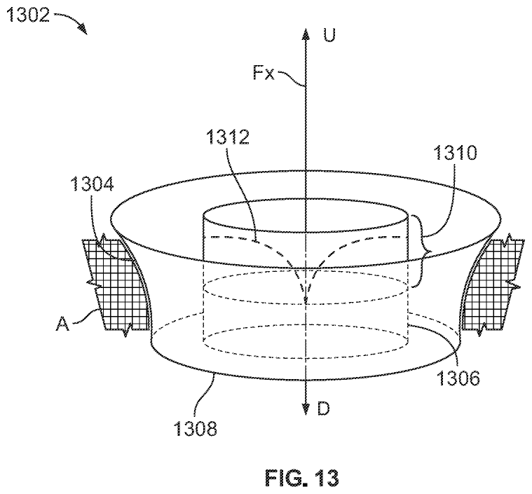

FIG. 13 shows illustrative prosthetic valve 1302 in valve annulus A. Valve 1302 may include one or more of the features that are shown or described in connection with FIGS. 2-12 and may have one or more features in common with one or more of the other valves shown or described herein. Valve 1302 may include fitting 1304. Fitting 1304 may include an inner wall 1306. Fitting 1304 may include an annular web 1308. Valve 1302 may include channel 1310. Channel 1310 may include leaflet 1312. Channel 1310 may define an axis parallel to axis Fx. Channel 1310 may have the same axis as axis Fx.

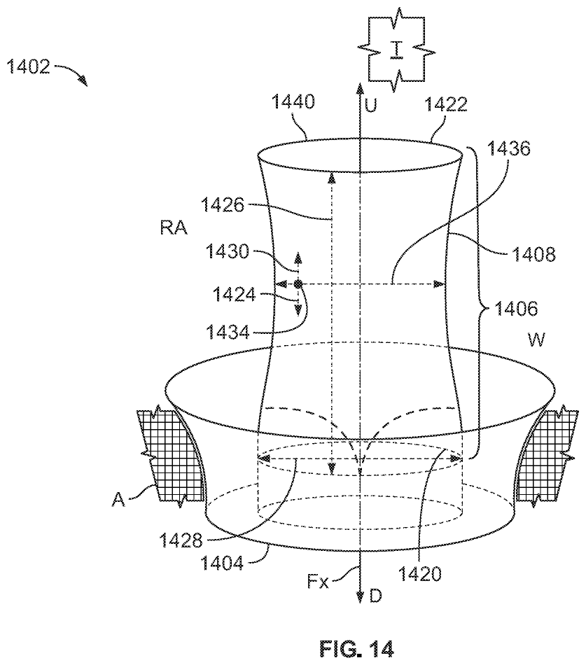

FIG. 14 shows illustrative prosthetic valve 1402 in valve annulus A. Valve 1402 may include one or more of the features that are shown or described in connection with FIGS. 2-13 and may have one or more features in common with one or more of the other valves shown or described herein. Valve 1402 may include fitting 1404. Valve 1402 may include brace 1406. Brace 1406 may include span 1408. Brace 1406 may include first end 1420. Brace 1406 may include second end 1422.

Span 1408 may have a length 1426. Span 1408 may have a width 1436. Width 1436 may be a minimum width of span 1408. Width 1436 may be a maximum width of span 1408. Width 1436 may be in between a minimum and maximum width for span 1408. Span 1408 may have different widths at different points along its length.

Width 1436 may smaller than inside diameter 1428. Width 1436 may larger than inside diameter 1428. Width 1436 may be equal to inside diameter 1428. Width 1436 may be ratio Ri of the inside diameter 1428. Smaller widths 1436 may increase a clearance between the span and the atrial wall, potentially providing a larger window to pass a device such as a catheter past valve 1402.

Width 1436 may be at a position on span 1408 between first end 1420 and second end 1422. Width 1436 may be a point along span 1408 such that in the upstream direction U and downstream direction D, the width of span 1408 is wider than at width 1436. Width 1436 may be constant along a segment 1430 of span 1408. Width 1436 may be located at a point along the length 1426 of the span 1408. If width 1436 is constant along segment 1430, then a center point of the segment may be used to indicate the width's location along span 1408. Fraction F may be a ratio of the length 1424 of the segment 1430 to the length 1426 of the span 1408. See Table 2.

Segment 1430 may have a center point 1434 that is in middle of the length of the segment. Height H is a ratio of the height of the center point 1434 in relation to the length 1426 of the span 1408. See Table 3.

Brace 1406 may have an outer surface 1440. In operation, some or all the outer surface 1440 may be in contact with atrial tissue.

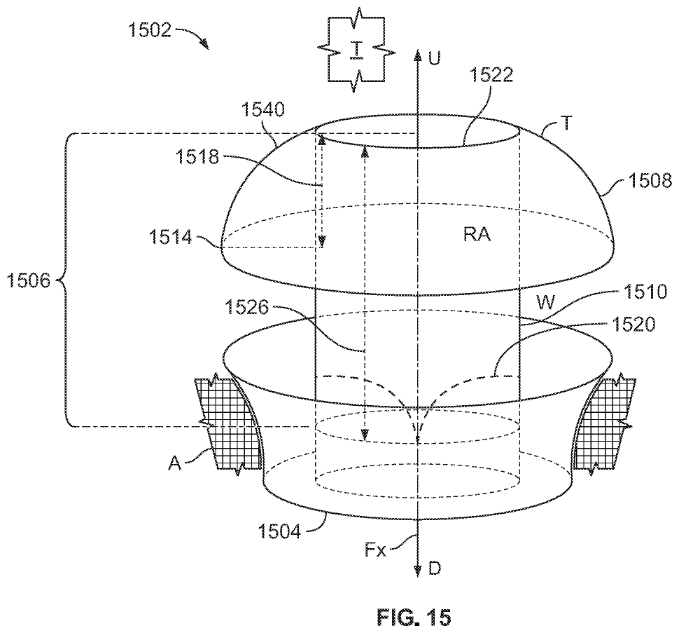

FIG. 15 shows illustrative prosthetic valve 1502 in valve annulus A. Valve 1502 may include one or more of the features that are shown or described in connection with FIGS. 2-14 and may have one or more features in common with one or more of the other valves shown or described herein. Valve 1502 may include fitting 1504. Valve 1502 may include brace 1506. Brace 1506 may include span 1510. Span 1510 may include artificial leaflet 1520. Brace 1506 may define a second end 1522 at the end of span 1510 at the latter's end along axis Fx in direction U. Brace 1506 may include support 1508. Brace 1506 may be configured to abut atrial tissue T. Brace 1506 may abut tissue T at the second end 1522. Support 1508 may abut T at its point furthest in direction U. Support 1508 may abut tissue T at any point along its surface.

One or more of fitting 1504, span 1510, and support 1508, alone or in combination, may define Window W.

Valve 1502 may contact the atrial tissue such that a window overlays an element of the CCS, such as a node. A window may prevent changing a rhythm of the heart's contraction. A window may provide clearance to pass through a device from one side of the right atrium to another side, clearing valve 1502. The device may be, for example, a catheter.

Support 1508 may extend from the second end 1522 to a lower edge 1514. Support 1508 may extend outward from the second end 1522 in a direction that is a direction that may not be parallel with axis Fx. Support 1508 may initially extend, relative to axis Fx, between 45° and perpendicular from the second end. As the support 1508 extends away from the second end 1522, support 1508 may curve in direction D, between 30° and 60°, relative to axis Fx. Finally, support 1508 may curve further in direction D, between 0° and 45°, relative to axis Fx.

Span 1510 may have a span length 1526. Support 1508 may have a support length 1518. Support length 1518 may be the distance, along axis Fx, from the second end 1522 to the edge 1514 of the support. The fraction of the support length 1518 to the span length 1526 is fraction I. See Table 10.

Brace 1506 may have an outer surface 1540. In operation, some or all the outer surface 1540 may be in contact with atrial tissue.

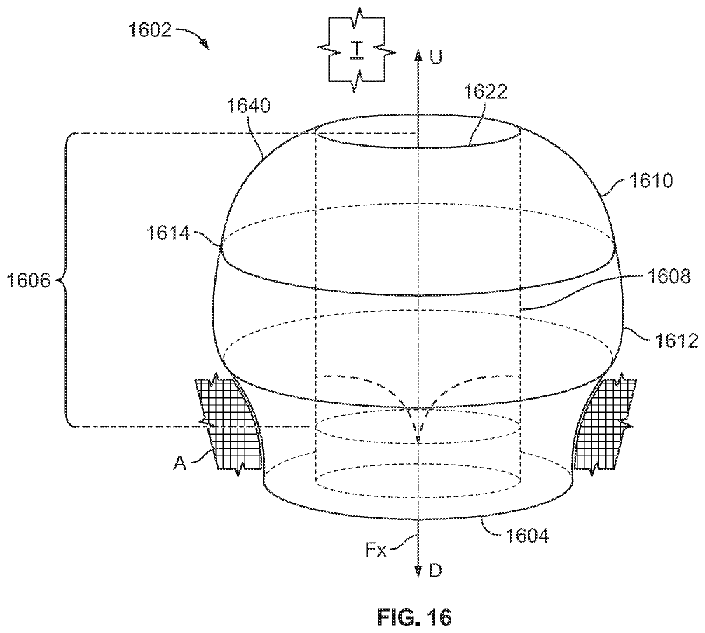

FIG. 16 shows illustrative prosthetic valve 1602 in valve annulus A. Valve 1602 may include one or more of the features that are shown or described in connection with FIGS. 2-14 and may have one or more features in common with one or more of the other valves shown or described herein.

Valve 1602 may include fitting 1604. Valve 1602 may include brace 1606. Brace 1606 may include span 1608. Brace 1606 may include a stand 1612. Brace 1606 may define a second end 1622 at the end of span 1608 at the latter's end along axis Fx in direction U. Brace 1606 may include support 1610. Brace 1606 may be configured to abut atrial tissue T. Brace 1606 may abut tissue T at the second end 1622. Support 1610 may abut tissue T at second end 1622. Support 1610 may abut tissue T at any point along its surface.

Support 1610 may extend from the second end 1622 to a lower edge 1614. Support 1610 may extend outward from the second end 1622 in a direction that is a direction that may not be parallel with axis Fx. Support 1610 may initially extend, relative to axis Fx, between 45° and perpendicular from the second end. As the support 1610 extends away from the second end 1622, support 1610 may curve in direction D, between 30° and 60°, relative to axis Fx. Finally, support 1610 may curve further in direction D, between 0° and 45°, relative to axis Fx.

Support 1610 may extend in downstream direction, away from span 1608, to stand 1612 and meet stand 1612 at edge 1614 of support. Stand 1612 may extend in direction U from fitting 1604.

Brace 1606 may have an outer surface 1640. In operation, some or all the outer surface 1640 may be in contact with atrial tissue.

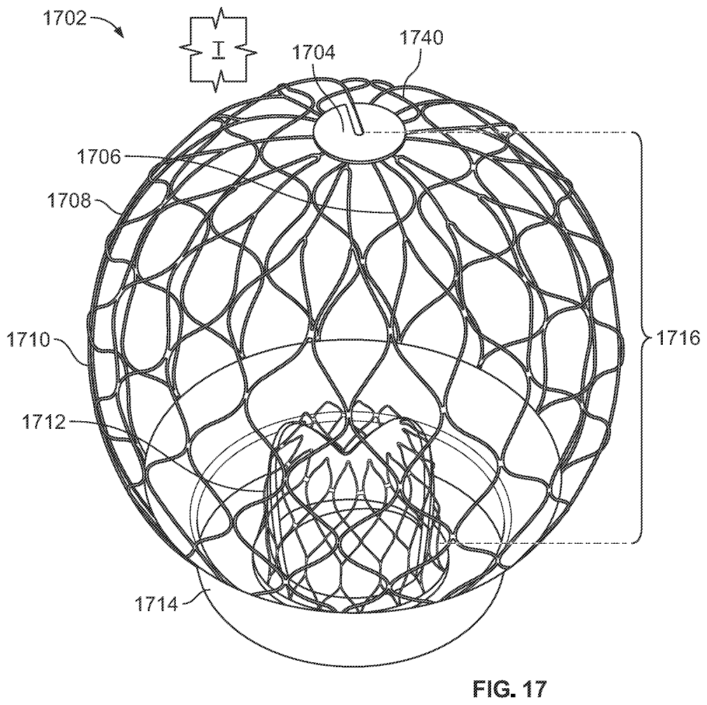

FIG. 17 shows illustrative prosthetic valve 1702. Valve 1702 may include one or more of the features that are shown or described in connection with FIGS. 2-14 and may have one or more features in common with one or more of the other valves shown or described herein. Valve 1702 may include fitting 1712. Valve 1702 may include brace 1716. Brace 1716 may include support 1708. Brace 1716 may include a cap 1704. Fitting 1712 made be made of mesh 1710. Fitting 1712 may be covered by skirt 1714. Brace 1716 may be made of mesh 1710. Mesh 1710 may be made of struts 1706.

Brace 1716 may have an outer surface 1740. In operation, some or all the outer surface 1740 may be in contact with atrial tissue.

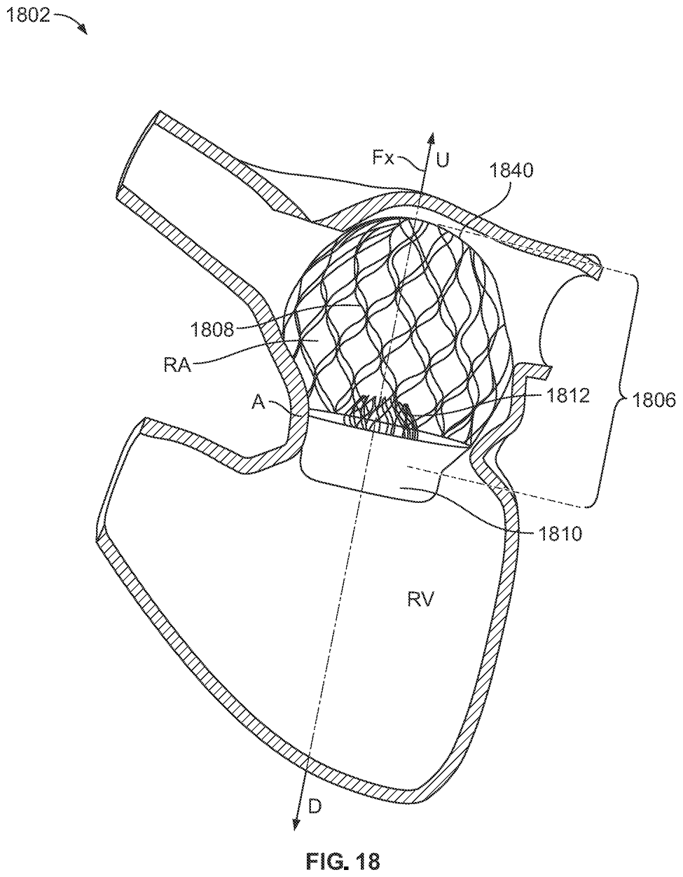

FIG. 18 shows illustrative prosthetic valve 1802 in right atrium RA and annulus A. Valve 1802 may include one or more of the features that are shown or described in connection with FIGS. 2-14 and may have one or more features in common with one or more of the other valves shown or described herein. Valve 1802 may be disposed in the right side of the heart (shown in posterior view). Valve 1802 may include fitting 1810. Valve 1802 may include channel 1812. Valve 1802 may include brace 1806. Brace 1806 may include support 1808. Support 1808 may contact the right atrium to provide a counter pressure for keeping fitting 1810 in annulus A.

Brace 1806 may have an outer surface 1840. In operation, some or all the outer surface 1840 may be in contact with atrial tissue.

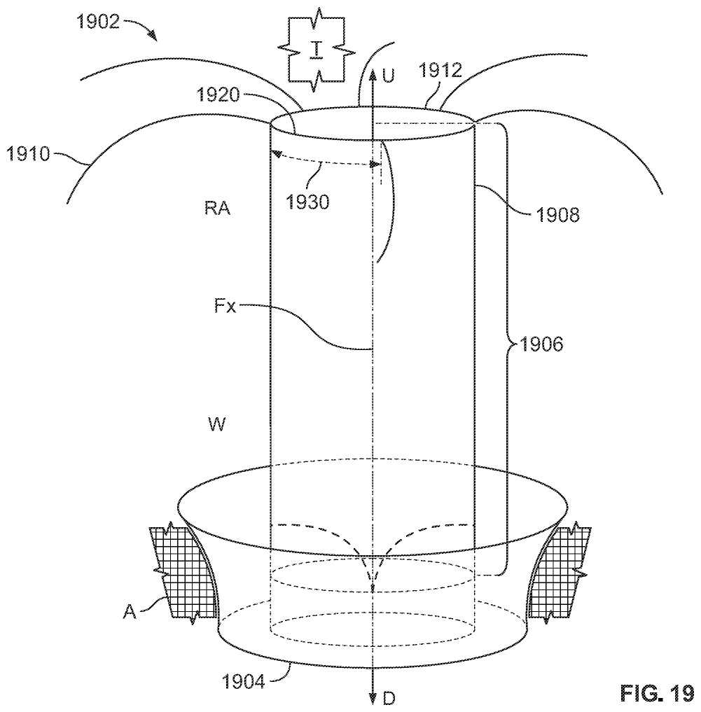

FIG. 19 shows illustrative prosthetic valve 1902 in valve annulus A. Valve 1902 may include one or more of the features that are shown or described in connection with FIGS. 2-14 and may have one or more features in common with one or more of the other valves shown or described herein. Valve 1902 may include fitting 1904. Valve may include brace 1906. Brace 1906 may include span 1908. Brace 1906 may define a second end 1912. Brace 1906 may include arms 1910 coming off span 1908 at the second end 1912. Arms 1910 may contact the right atrium to provide a counter pressure for keeping fitting 1904 in annulus A.

Arms 1910 may be disposed less than 180° arc length 1930, about axis Fx, away from each other. Values for arc length 1930 may be found in Table 11.

Arms 1910 may be one arm 1910. Arms 1910 may be more than one arm. Several arms 1910 may be used to distribute pressure around the tissue such as the right atrial tissue. Distributing the pressure may reduce or prevent changes to the heart beating rhythm.

One or more of fitting 1904, span 1908, and arms 1910, individually or together, may define a window. The window may provide clearance for a catheter to transit the right atrium.

Brace 1906 may have an outer surface 1920. In operation, some or all the outer surface 1920 may be in contact with atrial tissue.

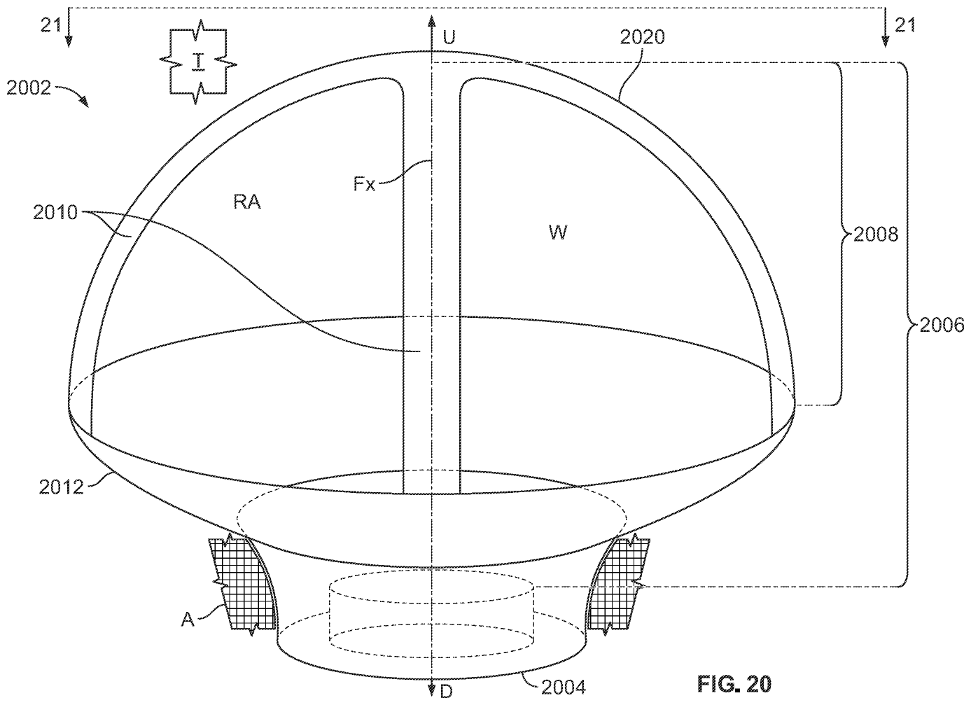

FIG. 20 shows illustrative prosthetic valve 2002 in valve annulus A. Valve 2002 may include one or more of the features that are shown or described in connection with FIGS. 2-14 and may have one or more features in common with one or more of the other valves shown or described herein. Valve 2002 may include fitting 2004. Valve 2002 may include brace 2006. Brace 2006 may include support 2008. Brace 2006 may include stand 2012. Brace 2006 may include arms 2010. Arms 2010 may extend to stand 2012. Arms 2010 may extend to fitting 2004. Stand 2012 and arms 2010 may define window W. Fitting 2004 and arms 2010 may define window W. Brace 2006 may include stand 2012.

The window may allow valve 2002 to contact the atrial tissue without contacting the cardiac conduction system (CC S). For example, the window may avoid contacting a hot spot of a node of the CCS. The window may avoid contacting a node of the CCS. The window may avoid contacting a node of the CCS along with a margin around the node. Avoiding contact with a node of the CCS may avoid changing a rhythm of contractions of the heart.

Arms 2010 may allow for distribution of pressure against T so that even if a node of the CCS is contacted, the node may not be stimulated to change its existing rhythm.

The window may provide clearance for an instrument to pass through such as a catheter. The device may pass through from an entrance of the atrium to an exit of the atrium.

Brace 2006 may have an outer surface 2020. In operation, some or all the outer surface 2020 may be in contact with atrial tissue.

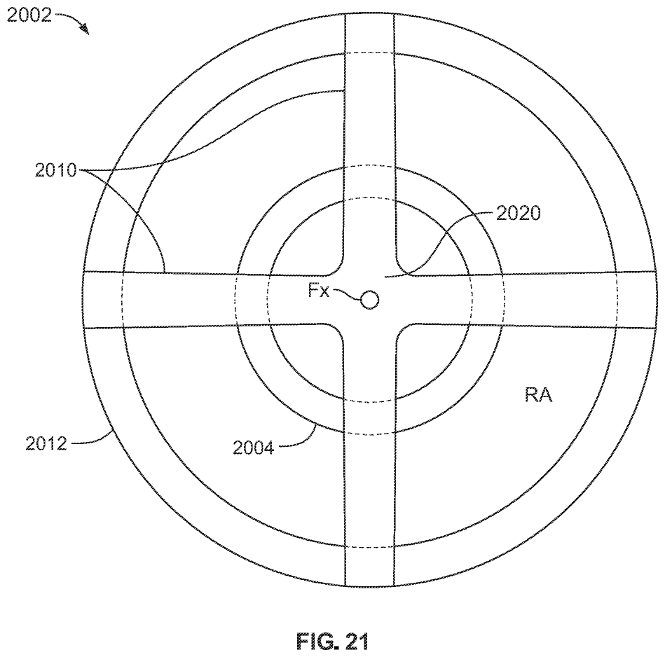

FIG. 21 shows prosthetic valve 2002, as viewed along view lines 21-21 (shown in FIG. 20).

Valve 2002 may include fitting 2004. Valve 2002 may include stand 2012. Valve 2002 may include arms 2010. Valve 2002 may include an outer surface 2020. This view may show how four arms 2010 may meet at central axis Fx in a direction U.

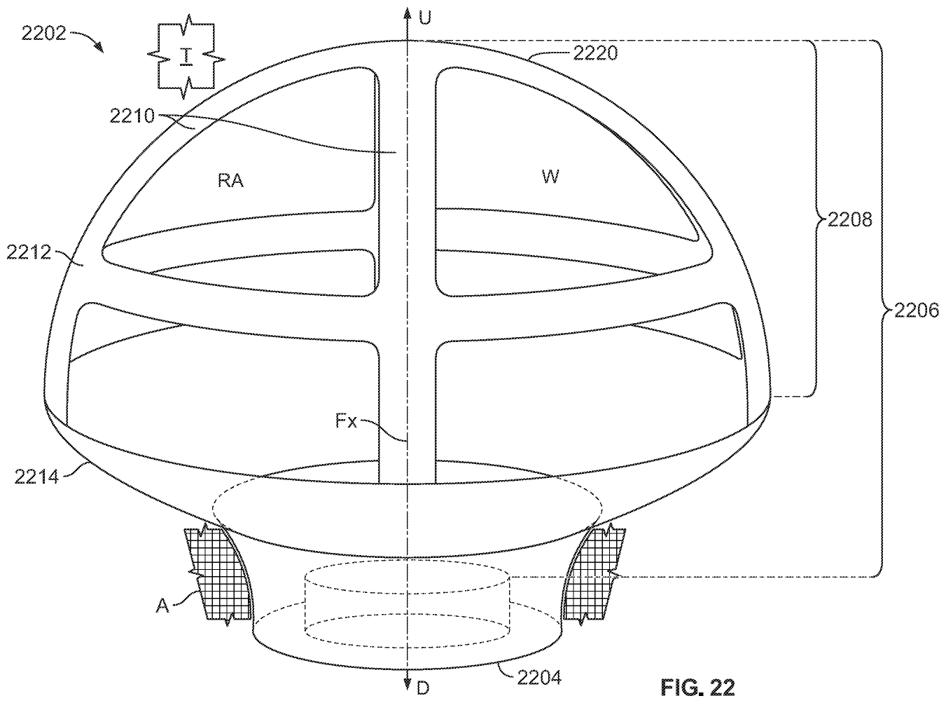

FIG. 22 shows illustrative prosthetic valve 2202 in valve annulus A. Valve 2202 may include one or more of the features that are shown or described in connection with FIGS. 2-14 and may have one or more features in common with one or more of the other valves shown or described herein. Valve 2202 may include fitting 2204. Valve 2202 may include brace 2206. Brace 2206 may include stand 2214. Brace 2206 may include support 2208. Brace 2206 may be configured to abut tissue T of right atrium RA.

Support 2208 may include arms 2210. Support 2208 may include circumferential stabilizer 2212. Arms 2210 may in operation bear a compressive force against T to press fitting 2204 into annulus A.

Circumferential stabilizer 2212 may be disposed on a plane which is perpendicular to axis Fx. Circumferential stabilizer 2212 may extend between arms 2210. Circumferential stabilizer 2212 may extend for 360°, in a direction of axis Fx, in a plane that is perpendicular to axis Fx. A combination of circumferential stabilizers 2212 and arms 2210 may extend for 360°, in a direction of axis Fx, in a plane that is perpendicular to axis Fx.

Stand 2214, circumferential stabilizer 2212, and arms 2210 may define window W. Window W may allow valve 2202 to contact atrial tissue T without contacting the CCS. For example, the window may avoid contacting a hot spot of a node of the CCS, a node of the CCS, or a node and a margin around the node of the CCS. Avoiding contact with a node of the CCS may avoid changing a rhythm of contractions of the heart.

Arms 2210 and circumferential stabilizer 2212 may allow for distribution of pressure against tissue T such that even if a node of the CCS is contacted, the node may not be stimulated to change its existing rhythm.

The window may be a clearance for a device to pass through such as a catheter. The device may pass through from an entrance of the atrium to an exit of the atrium.

Circumferential stabilizer 2212 may provide strength to valve 2202. Circumferential stabilizer 2212 may provide strength to valve 2202 and thereby allow for less material in arms 2210 than may be needed if circumferential stabilizer 2212 is not provided. Less material in the arms may provide for thinner arms which may allow for a larger window W.

Brace 2206 may have an outer surface 2220. In operation, some or all the outer surface 2220 may be in contact with atrial tissue.

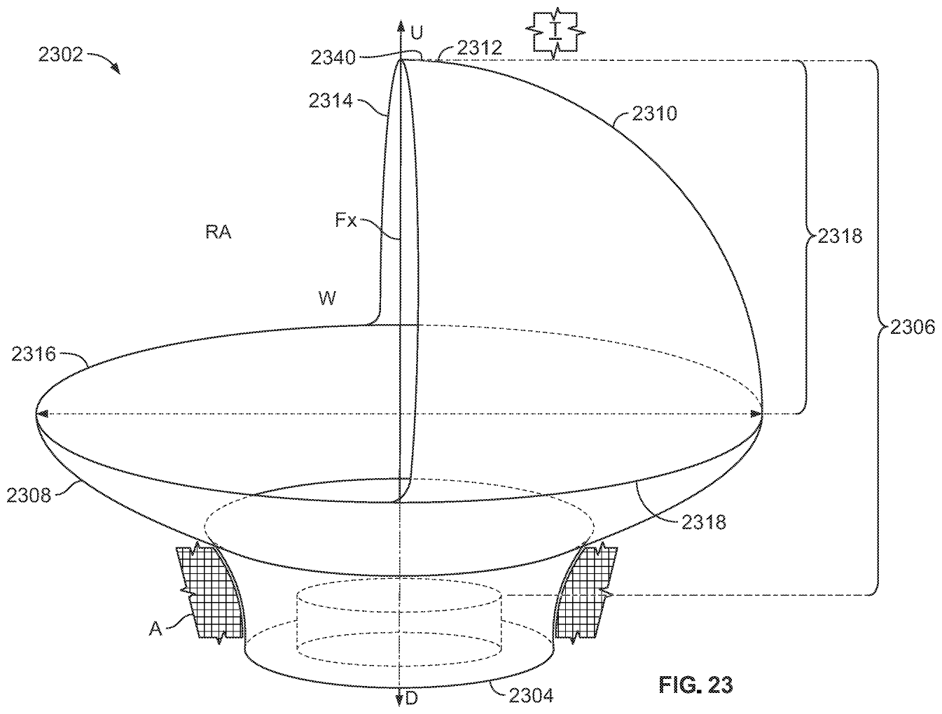

FIG. 23 shows illustrative prosthetic valve 2302 in valve annulus A. Valve 2302 may include one or more of the features that are shown or described in connection with FIGS. 2-14 and may have one or more features in common with one or more of the other valves shown or described herein. Valve 2302 may include fitting 2304. Valve 2302 may include brace 2306. Brace 2306 may include cage 2310. Brace 2306 may include support 2318. Cage 2310 and support 2318 may refer to the same structure. Brace 2306 may include stand 2308.

Support 2318 may define apex 2312. Apex 2312 may be the element of support 2318 that is furthest in direction U. Apex 2312 may abut atrial tissue T.

Support 2318 may define first edge 2314. Stand 2308 may define second edge 2316. When valve 2302 does not include a stand, fitting 2304 may define a second edge. First edge 2314 and second edge 2316 may define window W. Window W may be an opening in cage 2310.

The window may allow valve 2302 to contact the atrial tissue without contacting the CCS and thereby avoiding change in a rhythm of contractions of the heart.

The window may provide clearance for an instrument to pass through such as a catheter. The device may pass through from an entrance of the atrium to an exit of the atrium.

Brace 2306 may have an outer surface 2340. In operation, some or all the outer surface 2340 may be in contact with atrial tissue.

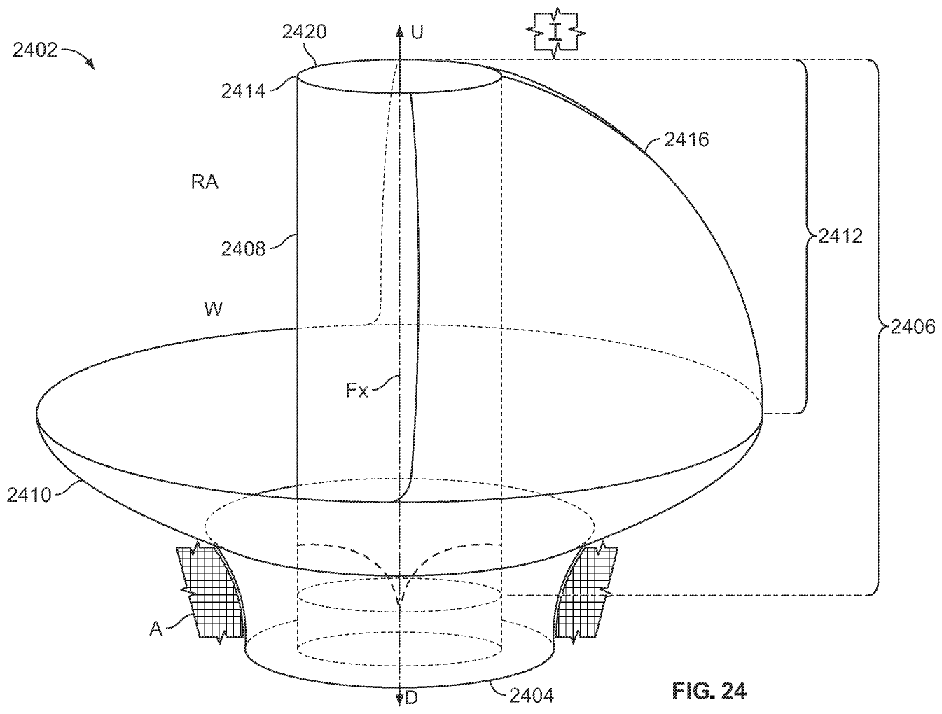

FIG. 24 shows illustrative prosthetic valve 2402 in valve annulus A. Valve 2402 may include one or more of the features that are shown or described in connection with FIGS. 2-14 and may have one or more features in common with one or more of the other valves shown or described herein. Valve 2402 may include fitting 2404. Valve 2402 may include brace 2406. Brace 2406 may include cage 2416. Brace 2406 may include support 2412. Support 2412 may include cage 2416. Brace 2406 may include stand 2410. Brace 2406 may include span 2408. Brace 2406 may include a second end 2414 at the top of span 2408 in direction U.

The second end 2414 may abut atrial tissue T.

Stand 2410, span 2408, and support 2412 may define window W. Window W may be an opening in cage 2416.

The window may allow valve 2402 to contact the atrial tissue without contacting the CCS and thereby avoiding change in a rhythm of contractions of the heart.

The window may provide clearance for an instrument to pass through such as a catheter. The device may pass through from an entrance of the atrium to an exit of the atrium.

Brace 2406 may have an outer surface 2420. In operation, some or all the outer surface 2420 may be in contact with atrial tissue.

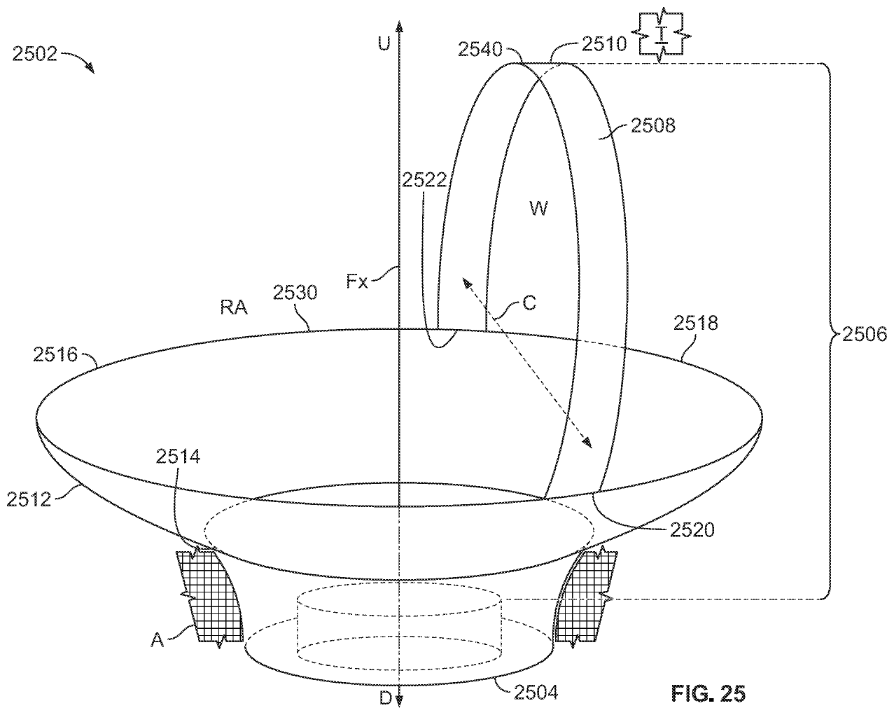

FIG. 25 shows illustrative prosthetic valve 2502 in valve annulus A. Valve 2502 may include one or more of the features that are shown or described in connection with FIGS. 2-14 and may have one or more features in common with one or more of the other valves shown or described herein. Valve 2502 may include fitting 2504. Fitting 2054 may define a rim on the surface of fitting 2504 in direction U. Valve 2502 may include brace 2506. Brace 2506 may include stand 2512. Brace 2506 may include arch 2508. Arch 2508 may be off-center from axis Fx.

Stand 2512 may support arch 2508 at a first base 2520. Stand 2512 may support arch 2508 at a second base 2522. Stand 2512 may define surface 2530 in direction U. Arch 2508 may define apex 2510. Apex 2510 may be the part of arch 2508 that is furthest in direction U. Apex 2510 may abut tissue T. Tissue T may be atrial tissue. Tissue T may be right atrial tissue.

The distance between the first base 2520 and the second base 2522 may be measured in circular arc, when the apparatus is in an unconstrained state, about the central axis. The larger circular arc may be called the major arc (MaA) 2516. The smaller circular arc may be called the minor arc (MiA) 2518. Values of MaA 2516 may be found in Table 12. Values of MiA 2518 may be found in Table 13.

Arch 2508 may provide window W. Areas around the arch may define window W. Window W may provide for valve 2502 to contact tissue T without directly contacting the CCS.

Window W may provide clearance C for a device to pass through arch 2508 such as a catheter.

Brace 2506 may have an outer surface 2540. In operation, some or all the outer surface 2540 may be in contact with atrial tissue.

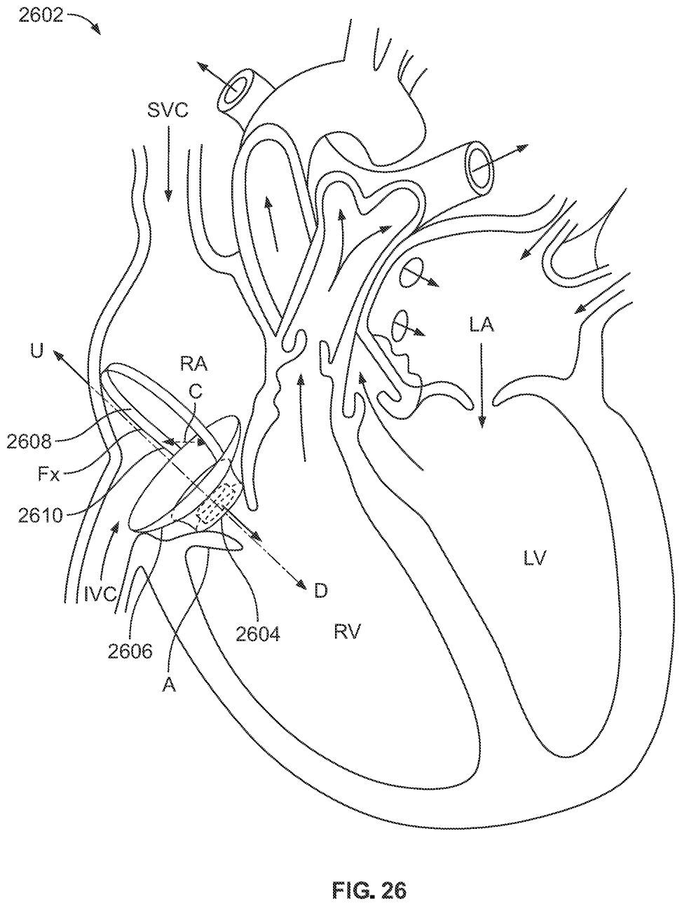

FIG. 26 shows illustrative prosthetic valve 2602 in the right atrium RA in the valve annulus A. Valve 2602 may include one or more of the features that are shown or described in connection with FIGS. 2-14 and may have one or more features in common with one or more of the other valves shown or described herein. Valve 2602 may include fitting 2604. Valve 2602 may include stand 2606. Valve 2602 may include arch 2608. Stand 2606 and arch 2608 may define window 2610. Window 2610 may provide for valve 2602 to contact tissue T without directly contacting the CCS. Window 2610 may provide an area for a device such as a catheter to pass through valve 2602. The catheter may pass through window 2610, between arch 2608 and atrial tissue, and avoid passing through arch 2608. The catheter may pass through window 2610 and clearance C in arch 2608.

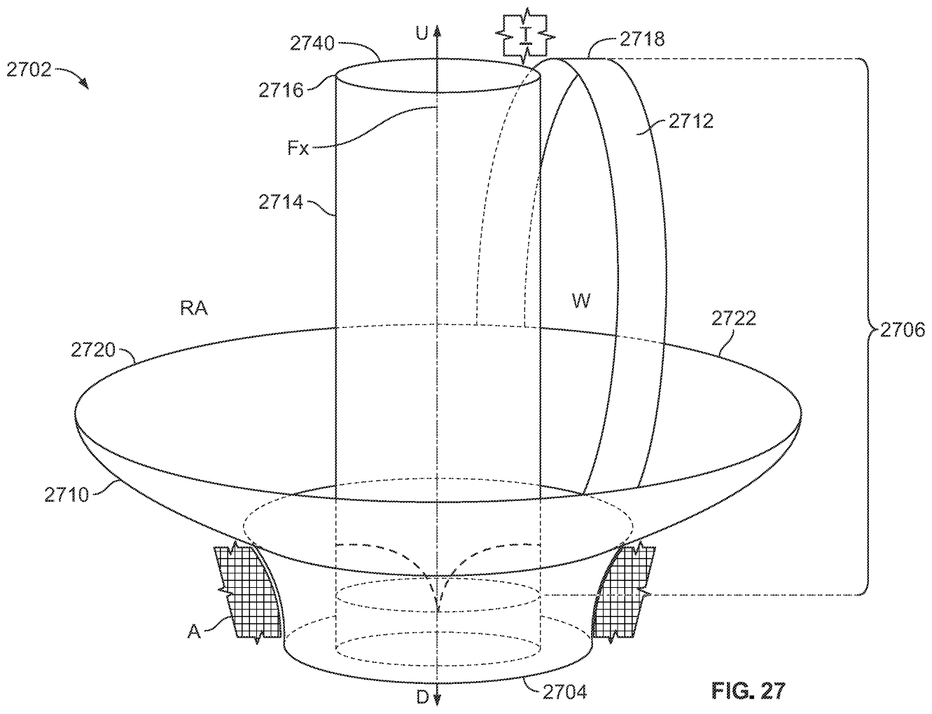

FIG. 27 shows illustrative prosthetic valve 2702 in valve annulus A. Valve 2702 may include one or more of the features that are shown or described in connection with FIGS. 2-14 and may have one or more features in common with one or more of the other valves shown or described herein. Valve 2702 may include fitting 2704. Valve 2702 may include brace 2706. Brace 2706 may include span 2714. Brace 2706 may include one or more such as arch 2712. Brace 2706 may include stand 2710.

Span 2714 may share axis Fx as a central axis. Span 2714 may have a second end 2716.

Arch 2712 may be off-center from axis Fx. Arch may define apex 2718.

When span 2714 and arch 2712 both contact tissue T, fitting 2704 may be secured more firmly in A than if only span 2714 or arch 2712 contacted tissue T. Span 2714 may contact tissue T at the second end 2716. Arch 2712 may contact tissue T at apex 2718.

The distance between a first base of arch 2712 and a second base of arch 2712 may be measured in circular arc, when the apparatus is in an unconstrained state, about the central axis. The larger circular arc may be called the major arc 2720. The smaller circular arc may be called the minor arc 2722.

One or more of stand 2710, arch 2712, and span 2714 and tissue T may provide window W. Window W may be upstream of the major arc in an area where span 2714 is not present. Window W may provide for valve 2702 to contact tissue T without directly contacting the CCS. The window may provide clearance for a device to pass through such as a catheter.

Brace 2706 may have an outer surface 2740. In operation, some or all the outer surface 2740 may be in contact with atrial tissue.

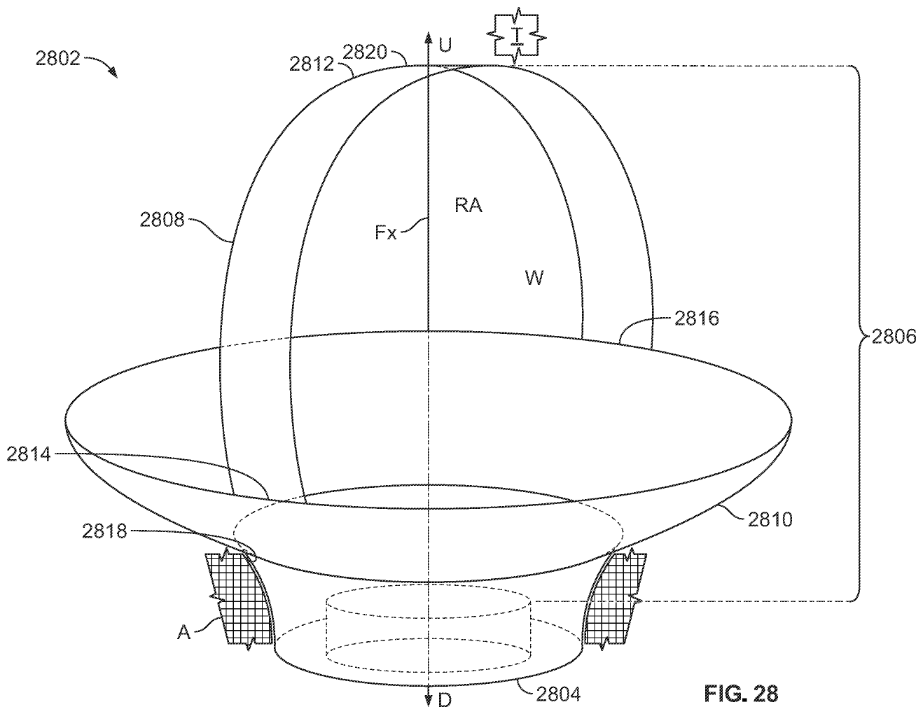

FIG. 28 shows illustrative prosthetic valve 2802 in valve annulus A. Valve 2802 may include one or more of the features that are shown or described in connection with FIGS. 2-14 and may have one or more features in common with one or more of the other valves shown or described herein. Valve 2802 may include fitting 2804. Fitting 2804 may define a rim 2818. Valve 2802 may include brace 2806. Brace 2806 may include stand 2810.

Brace 2806 may include arch 2808. Arch 2808 may meet stand 2810 at a first base 2814 and a second base 2816. Brace 2806 may include one or more arches such as arch 2808. Arch 2808 may define apex 2812. Apex 2812 may be a part of arch 2808 that is furthest in direction U. Arch 2808 may abut atrial tissue T. Apex 2812 may abut atrial tissue T. Tissue T may be right atrial tissue. Arch 2808 may intersect axis Fx. Arch 2808 may be centered with axis Fx. Apex 2812 may intersect axis Fx.

Window W may provide for the valve 2802 to contact tissue T without directly contacting the CCS. The window may provide clearance for a device to pass through such as a catheter.

Brace 2806 may have an outer surface 2820. In operation, some or all the outer surface 2820 may be in contact with atrial tissue.

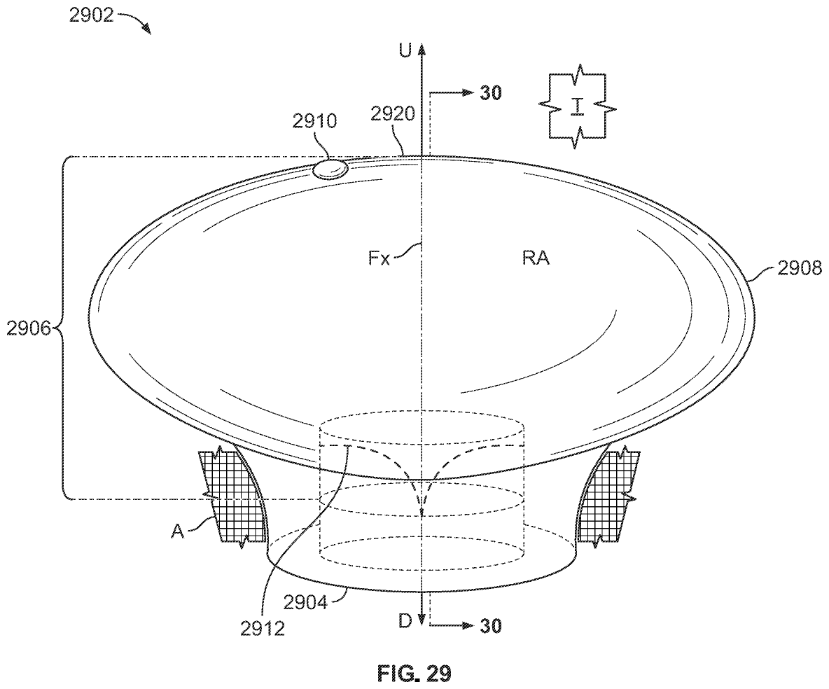

FIG. 29 shows illustrative prosthetic valve 2902 in valve annulus A. Valve 2902 may include one or more of the features that are shown or described in connection with FIGS. 2-14 and may have one or more features in common with one or more of the other valves shown or described herein. Valve 2902 may include fitting 2904. Valve 2902 may include brace 2906. Brace 2906 may include support 2908. Support 2908 may include cap 2910. Valve 2902 may include prosthetic leaflet 2912.

Support 2908 may have the shape of an ellipsoid. The ellipsoid may have a major axis. The ellipsoid may have a minor axis. The major axis and minor axis may be coplanar. The major axis and minor axis may lie on a plane which is perpendicular to axis Fx. The ratio of the major axis to the minor axis may be ratio S. See Table 14.

Support 2908 may be oriented to anchor in the right atrium such that one end of the major axis wedges into the IVC and the other end of the major axis wedges into the SVC. Wedged support 2908 may in operation bear a compressive force to press fitting 2904 into A.

Support 2908 may abut tissue T of right atrium RA. Support 2908 may abut part of tissue T of right atrium RA. Support 2908 may in operation bear a compressive force to press fitting 2904 into annulus A.

The mesh of support 2908 may be sized such that a window is formed around a node of the CCS.

The window may be sized such that a clearance is provided for a device to pass through such as a catheter.

Support 2908 may include cap 2910.

Brace 2906 may have an outer surface 2920. In operation, some or all the outer surface 2920 may be in contact with atrial tissue.

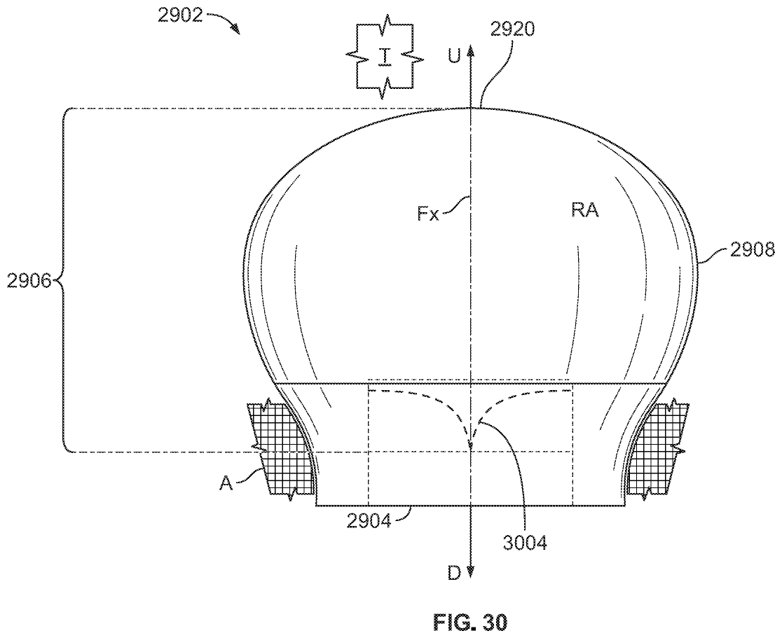

FIG. 30 shows prosthetic valve 2902, as viewed along view lines 30-30 (shown in FIG. 29). Valve 2902 may include fitting 2904. Valve 2902 may include brace 2906. Brace 2906 may include support 2908. Valve 2902 may include leaflet 3004. Valve 3102 may include outer surface 2920.

The mesh of support 2908 may be sized such that a window is formed around a node of the CCS. The window may be sized such that a clearance is provided for a device to pass through such as a catheter. The shape of 2902 along view lines 30-30 may show a narrower width along the minor axis of the ellipsoid.

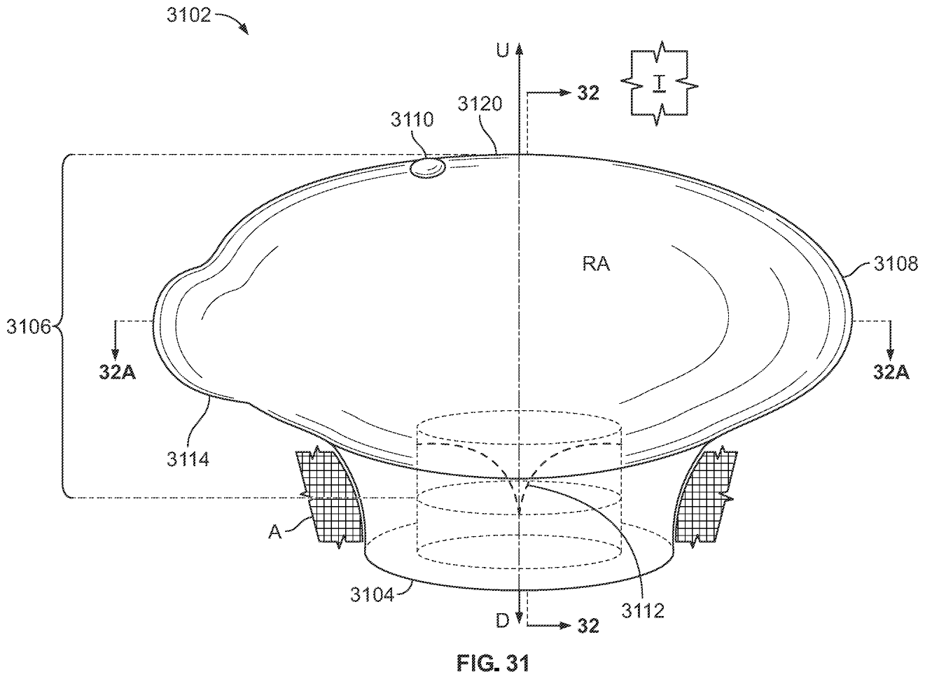

FIG. 31 shows illustrative prosthetic valve 3102 in valve annulus A. Valve 3102 may include one or more of the features that are shown or described in connection with FIGS. 2-14 and may have one or more features in common with one or more of the other valves shown or described herein. Valve 3102 may include fitting 3104. Valve 3102 may include brace 3106. Brace 3106 may include support 3108. Support 3108 may include cap 3110. Valve 3102 may include leaflet 3112.

Support 3108 may have the shape of an ellipsoid. The ellipsoid may have a major axis. The major axis may be perpendicular to axis Fx. The ellipsoid may have a minor axis. The minor axis may be coplanar with axis Fx. Support 3108 may include protrusion 3114. Protrusion 3114 may help valve 3102 to sit within the right atrium RA. Protrusion 3114 may help fitting 3104 to sit within annulus A. Protrusion 3114 may be seated in the IVC. Protrusion 3114 may be seated in the SVC. A second protrusion may be on the opposite side of the major axis. Two protrusions on opposite sides of the major axis may allow for the protrusions to be seated in the IVC and SVC.

Support 3108 may be oriented to anchor in the right atrium such that one end of the major axis wedges into the IVC and the other end of the major axis wedges into the SVC. Wedged support 3108 may in operation bear a compressive force to press fitting 2904 into A.

The mesh of support 3108 may be sized such that a window is formed around a node of the CCS.

The window may be sized such that a clearance is provided for a device to pass through such as a catheter.

Brace 3106 may have an outer surface 3120. In operation, some or all the outer surface 3120 may be in contact with atrial tissue.

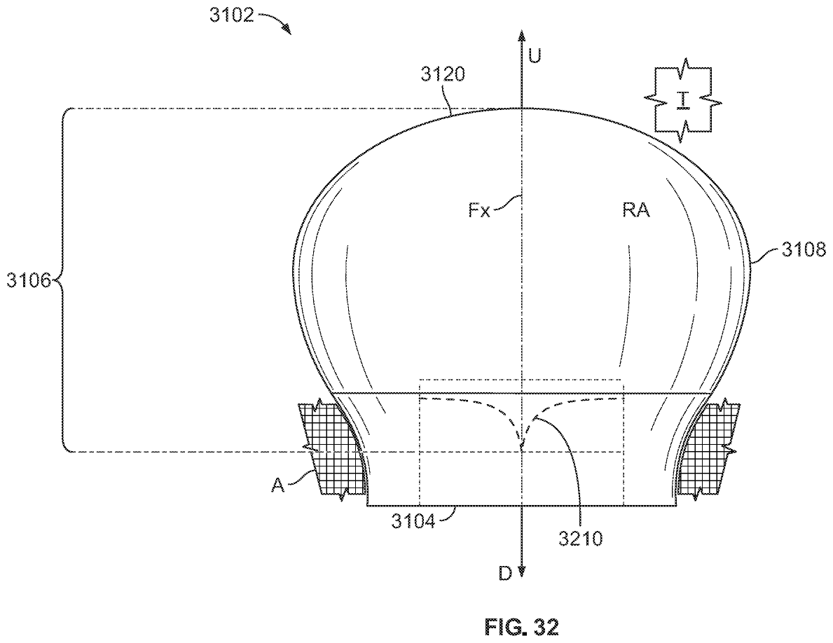

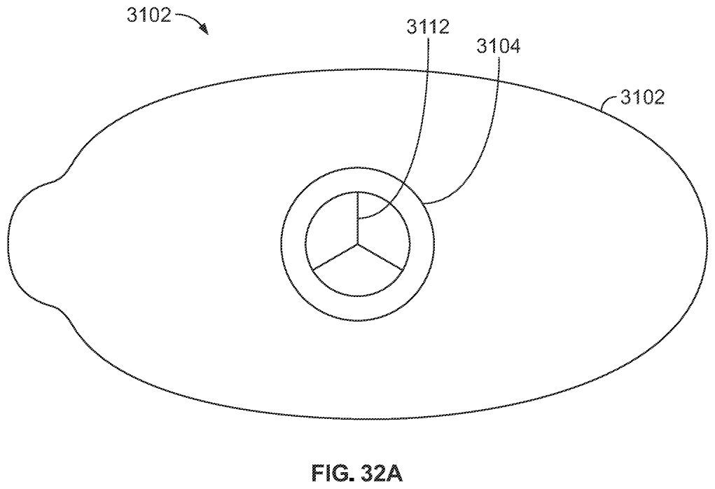

FIG. 32 shows prosthetic valve 3102, as viewed along view lines 32-32 (shown in FIG. 31). Valve 3102 may include fitting 3104. Valve 3102 may include brace 3106. Brace 3106 may include support 3108. Valve 3102 may include leaflet 3210. Valve 3102 may include outer surface 3120.

The mesh of support 3108 may be sized such that a window is formed around a node of the CCS. The window may be sized such that a clearance is provided for a device to pass through such as a catheter. The shape of 3102 along view lines 32-32 may show a narrower width along the minor axis of the ellipsoid.

FIG. 32A shows prosthetic valve 3102, as viewed along view lines 32A-32A (shown in FIG. 31). Valve 3102 may include fitting 3104. Valve 3102 may include support 3108. Valve 3102 may include leaflet 3112.

The mesh of support 3108 may be sized such that a window is formed around a node of the CCS. The window may be sized such that a clearance is provided for a device to pass through such as a catheter. The shape of 3102 along view lines 32A-32A may show a narrower width along the minor axis of the ellipsoid and a larger protrusion on the left side than the right side. The larger protrusion may wedge into the IVC while the smaller protrusion may wedge into the SVC. The larger protrusion may wedge into the SVC while the smaller protrusion may wedge into the IVC. Wedging may assist in keeping valve 3102 secured and fitting 3104 in a location in annulus A.

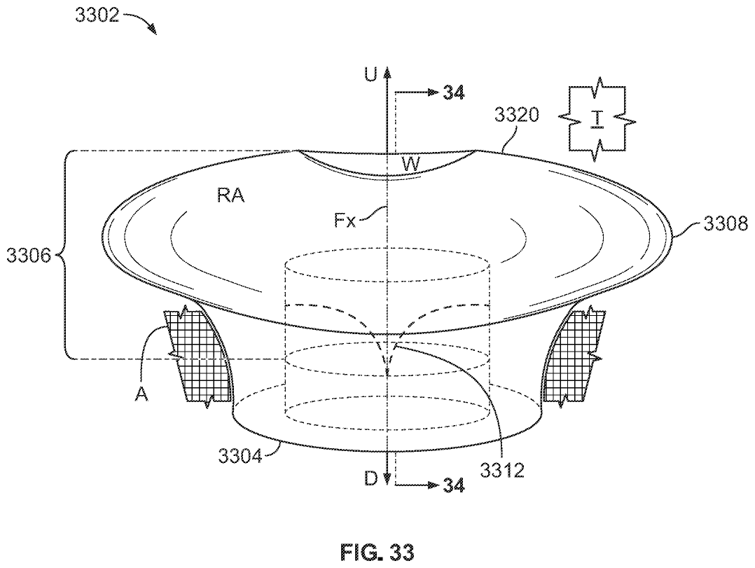

FIG. 33 shows illustrative prosthetic valve 3302 in valve annulus A. Valve 3302 may include one or more of the features that are shown or described in connection with FIGS. 2-14 and may have one or more features in common with one or more of the other valves shown or described herein. Valve 3302 may include fitting. Valve 3302 may include brace 3306. Brace 3306 may include support 3308. Valve 3302 may include leaflet 3312.

Support 3308 may have the shape of an ellipsoid. The ellipsoid may have a cavity at P. The ellipsoid may have a major axis. The major axis may be perpendicular to axis Fx. The ellipsoid may have a minor axis. The minor axis may be parallel to axis Fx. The ratio of the major axis to the minor axis may be 0.7×.

One of the ends of support 3308 along the major axis may be oriented to wedge into the IVC. One of the ends of support 3308 along the major axis may be oriented to wedge into the SVC. Support 3308 may in operation bear a compressive force to press fitting 3304 into annulus A.

Support 3308 may abut tissue T of right atrium RA. Support 3308 may abut part of tissue T of right RA. Support 3308 may in operation bear a compressive force to press fitting 3304 into annulus A.

Window W may be positioned to include a node of the CCS. The mesh of support 3308 may be sized such that a window is formed around a node of the CCS.

The mesh may be sized such that a clearance is provided for a device to pass through such as a catheter. When one of the ends of supports 3308 along the latter's major axis is wedged into the IVC, a catheter may be passed through the IVC and through mesh in support 3308. The catheter may then emerge from window W of support 3308 and continue through the right atrium into the SVC.

When one of the ends of support 3308 along the latter's major axis is wedged into the SVC, a catheter may be passed through the IVC and through window W in support 3308. The catheter may then emerge through the mesh of the wedged end of support 3308 and continue through the right atrium into the SVC.

Brace 3306 may have an outer surface 3320. In operation, some or all the outer surface 3320 may be in contact with atrial tissue.

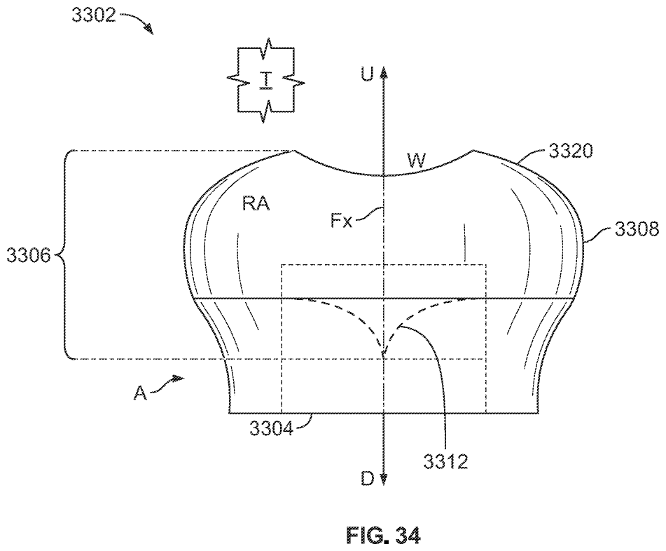

FIG. 34 shows prosthetic valve 3302, as viewed along view lines 34-34 (shown in FIG. 33). Valve 3302 may include fitting 3304. Valve 3302 may include brace 3306. Brace 3306 may include support 3308. Valve 3302 may include leaflet 3312. Valve 3302 may include outer surface 3320.

The mesh of support 3308 may be sized such that a window is formed around a node of the CCS. The window may be sized such that a clearance is provided for a device to pass through such as a catheter. The shape of 3302 along view lines 34-34 may show a narrower width along the minor axis of the ellipsoid and a similar shape to window W.

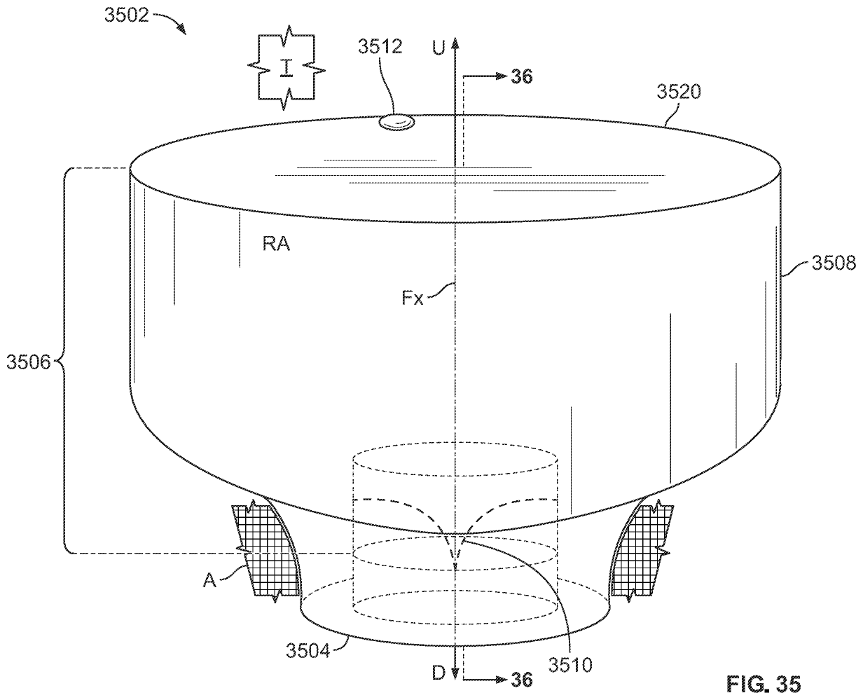

FIG. 35 shows illustrative prosthetic valve 3502 in valve annulus A. Valve 3502 may include one or more of the features that are shown or described in connection with FIGS. 2-14 and may have one or more features in common with one or more of the other valves shown or described herein. Valve 3502 may include fitting 3504. Valve 3502 may include brace 3506. Brace 3506 may include support 3508. Support 3508 may include cap 3512. Valve 3502 may include leaflet 3510.

Support 3508 may have an ovular shape along axis Fx in the part of valve 3502 in direction U. The dimensions of the oval may stay constant with respect to distance from axis Fx in direction U. Support 3508 may have an elliptical shape along axis Fx in the part of valve 3502 in direction D. The dimensions of the ellipse may decrease in direction D until support 3508 meets fitting 3504. Support 3508 in direction D may have a similar shape and size as an outside wall of fitting 3504 in direction U.

Support 3508 may abut T of RA. Support 3508 may abut part of T of RA. Support 3508 may in operation bear a compressive force to press fitting 3504 into A.

The mesh of support 3508 may include a window. The window may be sized such that a window is formed around a node of the CCS.

The window may be sized such that a clearance is provided for a device to pass through such as a catheter.

Brace 3506 may have an outer surface 3520. In operation, some or all the outer surface 3520 may be in contact with atrial tissue.

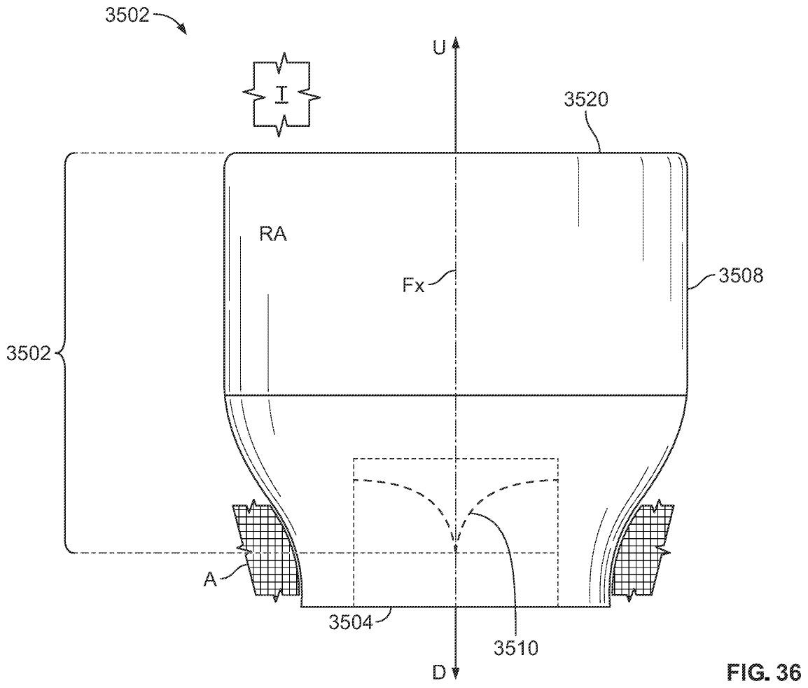

FIG. 36 shows prosthetic valve 3502, as viewed along view lines 36-36 (shown in FIG. 35). Valve 3502 may include one or more of the features that are shown or described in connection with FIGS. 2-14 and may have one or more features in common with one or more of the other valves shown or described herein. Valve 3502 may include fitting 3504. Valve 3502 may include brace 3506. Brace 3506 may include support 3508. Valve 3502 may include leaflet 3510. Valve 3502 may include outer surface 3520.

Support 3508 may abut tissue T of RA. Support 3508 may abut part of tissue T of RA. Support 3508 may in operation bear a compressive force to press fitting 3504 into A.

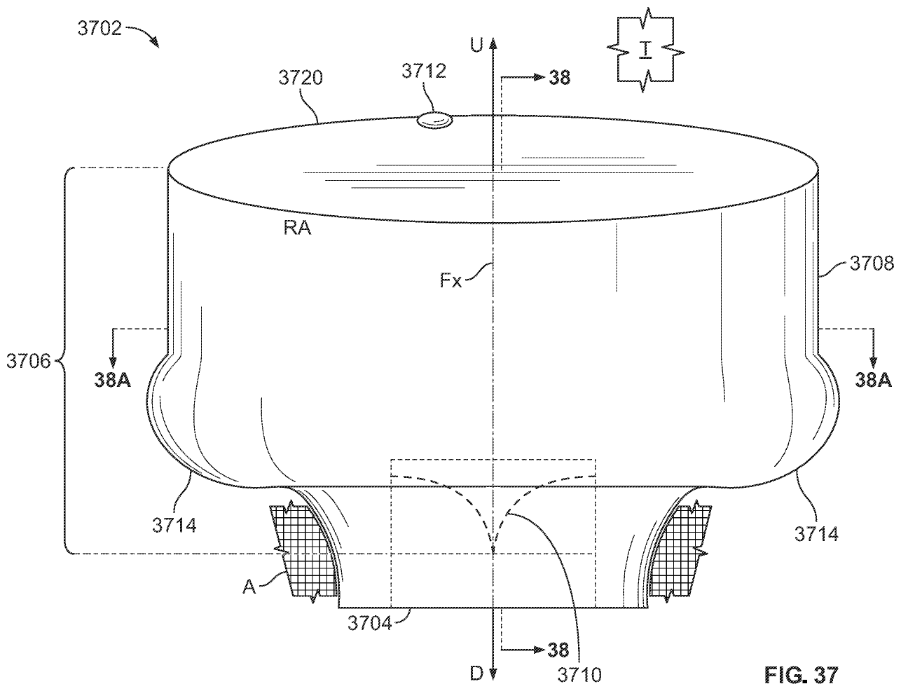

FIG. 37 shows illustrative prosthetic valve 3702 in valve annulus A. Valve 3702 may include one or more of the features that are shown or described in connection with FIGS. 2-14 and may have one or more features in common with one or more of the other valves shown or described herein. Valve 3702 may include fitting 3704. Valve 3702 may include brace 3706. Brace 3706 may include support 3708. Support 3708 may include cap 3712. Valve 3702 may include leaflet 3710.

Support 3708 may have an ovular shape along axis Fx in the part of valve 3502 in direction U. The dimensions of the oval may stay constant with respect to distance from axis Fx in direction U. Support 3708 may have an elliptical shape along axis Fx in the part of valve 3702 in direction D. The dimensions of the eclipse may decrease in direction D until support 3708 meets fitting 3704. Support 3708 in direction D may have a similar shape and size as an outside wall of fitting 3704 in direction U.

Protrusions 3714 may be seated in the IVC and the SVC. Seating protrusions 3714 in the IVC and SVC may help secure fitting 3704 in annulus A.

Two protrusions on opposite sides of the major axis may allow for the protrusions to be seated in the IVC and SVC.

Valve 3702 may contain one protrusion 3714. The one protrusion 3714 may seat in the IVC. The one protrusion 3714 may seat in the SVC. The one protrusion 3714 may help secure fitting 3704 in annulus A.

Support 3708 may abut tissue T of right atrium RA. Support 3708 may abut part of tissue T of right atrium RA. Support 3708 may in operation bear a compressive force to press fitting 3704 into A.

The mesh of support 3708 may be sized such that a window is formed around a node of the CCS.

The window may be sized such that a clearance is provided for a device to pass through such as a catheter.

Brace 3706 may have an outer surface 3720. In operation, some or all the outer surface 3720 may be in contact with atrial tissue.

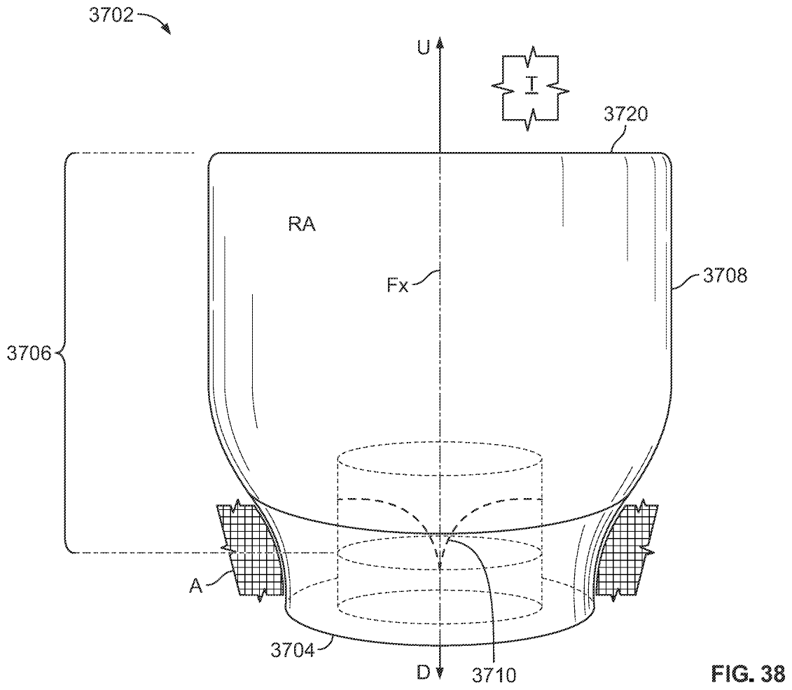

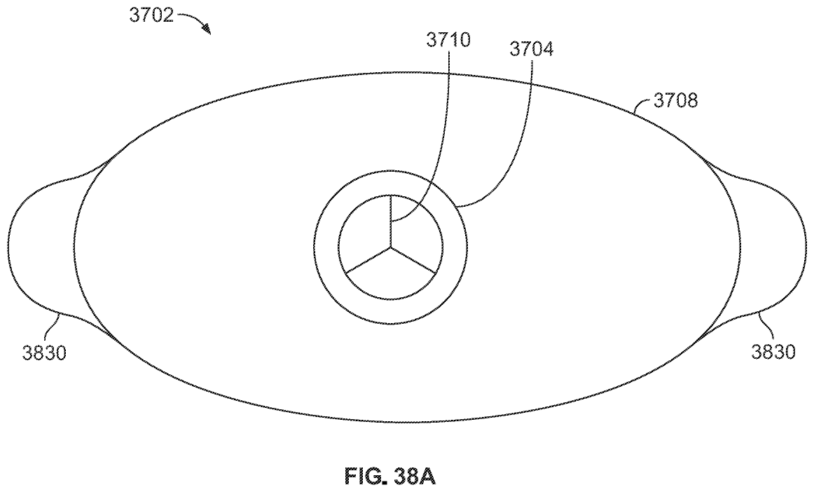

FIG. 38 shows prosthetic valve 3702, as viewed along view lines 38-38 (shown in FIG. 37). Valve 3702 may include fitting 3704. Valve 3702 may include brace 3706. Brace 3706 may include support 3708. Valve 3702 may include leaflet 3710. Valve 3702 may include outer surface 3720.

Support 3708 may abut tissue T of right atrium RA. Support 3708 may abut part of tissue T of right atrium RA. Support 3708 may in operation bear a compressive force to press fitting 3704 into annulus A.

FIG. 38A shows prosthetic valve 3702, as viewed along view lines 38A-38A (shown in FIG. 37). Valve 3702 may include fitting 3704. Valve 3702 may include support 3708. Valve 3702 may include leaflet 3710.

Support 3708 may have the shape of an ellipsoid along view lines 38A-38A. The ellipsoid may have a major axis and a minor axis. Protrusions 3830 may extend along the major axis of support 3708. Protrusions 3830 may wedge into the IVC and SVC, which may help secure fitting 3704 in the annulus.

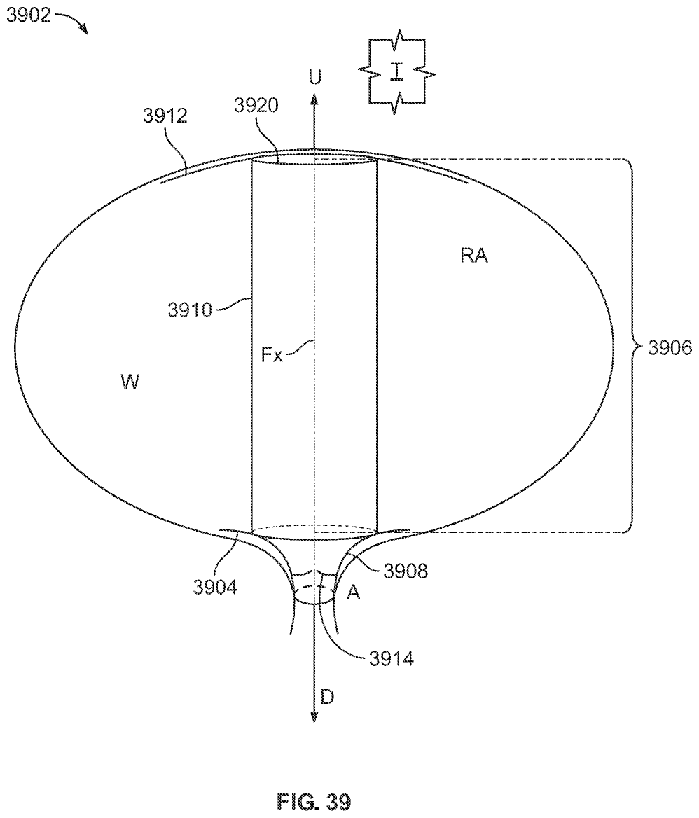

FIG. 39 shows illustrative prosthetic valve 3902 in valve annulus A. Valve 3902 may include one or more of the features that are shown or described in connection with FIGS. 2-14 and may have one or more features in common with one or more of the other valves shown or described herein. Valve 3902 may include fitting 3908. Fitting 3908 may be a lower anchor. Fitting 3908 may have one or more lower arms 3904. Lower arms 3904 of fitting 3908 may contact A. Lower arms 3904 of fitting 3908 may contact the tissue above A in direction U. Lower arms 3904 may help provide anchoring against tissue T. Lower arms 3904 may help provide anchoring against annulus A.

Fitting 3908 may include prosthetic leaflets 3914. Valve may include brace 3906. Brace 3906 may include span 3910. Brace 3906 may define a second end. Brace 3906 may include one or more upper arms 3912 coming off span 3910 at the second end. Upper arms 3912 may contact the right atrium to provide a counter pressure for keeping fitting 3908 in annulus A.

Upper arms 3912 may be disposed less than 180°, about axis Fx, away from each other.

Several upper arms 3912 may be used to distribute pressure around the tissue such as the right atrial tissue. Distributing the pressure may reduce or prevent changes to the heart beating rhythm.

Fitting 3908, lower arms 3904, span 3910, and upper arms 3912 may define a window. The window may provide clearance for a catheter to pass by as it is transmitting the right atrium.

Upper arms 3912 may be biased to flex upward in direction U. Upper arms 3912 may be deformed to flex upward. Upper arms 3912 in such a deformed configuration may provide force against the upper portion of tissue T as the arms seek to return to an undeformed configuration. The lower anchor structure may be biased to rotate in direction D. The lower anchor structure may be deformed to provide force against annulus A or tissue above annulus A going into the right atrium RA in direction U. This lower anchor structure in such a deformed configuration may provide force against the lower portion of the right atrium RA tissue as the lower anchor structure seeks to return to an undeformed configuration.

Similar to the valves beforehand, valve 3902 may be configured for translation through a lumen of a delivery tube in a collapsed configuration and delivery to the subject heart chamber.

Brace 3906 may have an outer surface 3920. In operation, some or all the outer surface 3920 may be in contact with atrial tissue.

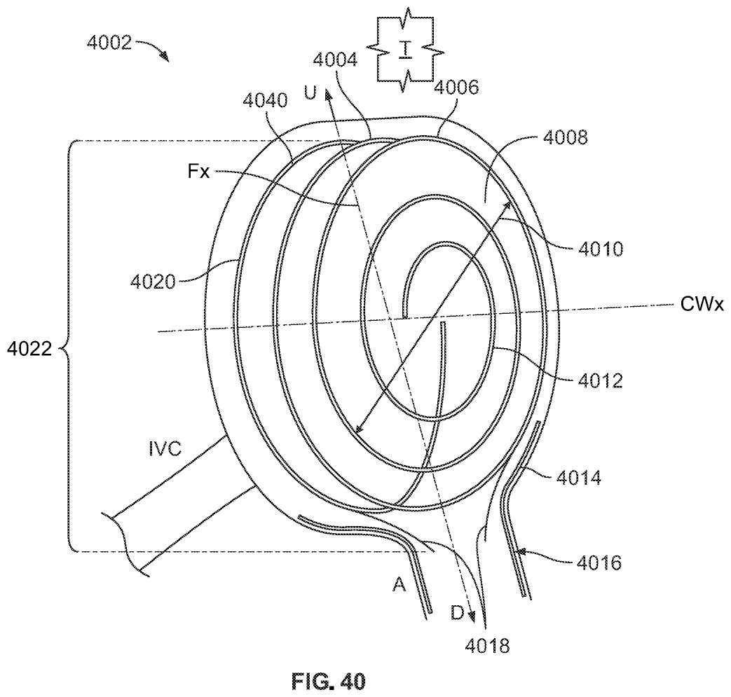

FIG. 40 shows illustrative prosthetic valve 4002 in valve annulus A. Valve 4002 may include one or more of the features that are shown or described in connection with FIGS. 2-14 and may have one or more features in common with one or more of the other valves shown or described herein. Valve 4002 may include fitting 4016. Fitting 4016 may support valve 4002 in a tricuspid annulus. Fitting 4016 may include a valve support extension. Fitting 4016 may include leaf support 4014. Valve 4002 may include brace 4022. Brace 4022 may include prosthetic leaflets 4018. Brace 4022 may include anchor 4008. Brace 4022 may include coiled wire 4012. Coiled wire 4012 may include inner coil 4020. Coiled wire may include intermediate coil 4004. Coiled wire may include outer coil 4006.

Coiled wire 4012 may define an axis CWx. Coiled wire may have a width 4010. Width 4010 may be a width of the coiled wire in expanded state. Width 4010 may be measured in a perpendicular to CWx. Axis Fx may be perpendicular to CWx.

Brace 4022 and anchor 4008 may be deployed to be expanded against anatomy such as a RA. RA may limit expansion of coiled wire 4012. Coiled wire 4012 may thus be biased against the anatomy.

Coiled wire 4012 may expand in at least one, and as many as three spatial dimensions, e.g., height H, depth D, and/or width W. In coiled wire 4012, outer coil 4006 may be spaced apart from intermediate coil 4004 which may be spaced apart from a second outer coil when expanded. Outer coil 4006 and second outer coils may be on opposing sides of intermediate coil 4004 and may be of substantially the same diameter around CWx when expanded. Inner coil 4020 may have a smaller diameter than the other coils.

The number of wire coils of the same diameter may be greater or less than three. A smaller interior intermediate coil may be present to achieve the goals of the present disclosure.

The apparatus may include an outer and an inner wire coil that are of effectively the same diameter when expanded or in any collapsed configuration (and therefore may contribute to the minimum depth when collapsed) and one or more inner wire coils that are of a smaller diameter than the two outer wires (and therefore may not contribute to the minimum depth when collapsed). One or more intermediate coils may be of larger diameter than the first inner and first outer coils.

A first outer coil may have a diameter that is substantially the same as a second outer coil. One or more intermediate coils may be in spaced-apart disposition with each other and with the first and second outer coils. The intermediate coils, if more than one, may have diameters that allow nesting within the intermediate coils and within the first and second outer coils. In this case, the minimum depth at collapse may be the cumulative thickness of the first and second outer coil. If one intermediate coil is included, the diameter may be smaller, or larger, than the first and/or second outer coil to allow a nested collapsing.

A collapsing configuration may be achieved in a first spatial dimension, followed by collapsing in two additional, different spatial dimensions.

The anchor section is illustrated as a substantially circular coiled wire profile when expanded. This configuration allows reduction of dimensions height and width to a collapsed configuration at substantially the same time and rate. Other shapes, e.g., cylindrical, or eccentric/asymmetric expanded forms may also be employed as the skilled artisan will recognize.

An expanded configuration of anchor section may be provided to provide radial forces that may essentially oppose each other when anchor section presses against the tissue of the heart chamber and may thereby anchor the device in place for subsequent endothelization.

An illustrative prosthetic leaflet support is shown in FIG. 40 as disposed at, or near, the downstream region of the anchor section. The prosthetic leaflet support may include a valve support including one or more extensions (extending away from the anchor region in a downstream direction) that may be individually extending elements in some embodiments or that may include a cylinder or other fully or partially closed extension. The prosthetic leaflets may be attached on an inner side of the valve support extension(s). The valve support extension(s) may be used to assist in orienting the prosthetic heart valve for full expansion and anchoring within the heart chamber.

The length of the valve support extension away, and downstream, from the anchor section may ensure that the valve support extension(s) do not interfere with the natural functioning of the native leaflets.

The length of the valve support extension away, and downstream, from the anchor section may ensure that the prosthetic leaflets do not interfere with the natural functioning of the native leaflets. This may preserve functionality in the native leaflets while supplementing valve function.

The valve support extension(s) may include a length that engages and modifies, reduces, or even prevents, the native leaflets from functioning.

The prosthetic leaflets may be located at any point along the valve support extension(s).

Valve support extensions may be included. The prosthetic leaflets may be attached at the downstream region of the anchor, either directly to the anchor wire or via an attachment element. Both may be at substantially the same level as the downstream region of the anchor. The anchor section may be oriented to implant wherein the prosthetic leaflets are positioned or located above the annulus.

Valve 4002 may be configured for translation through a lumen of a delivery tube in a collapsed configuration and delivery to the subject heart chamber.

Brace 4006 may have an outer surface 4040. In operation, some or all the outer surface 4040 may be in contact with atrial tissue.

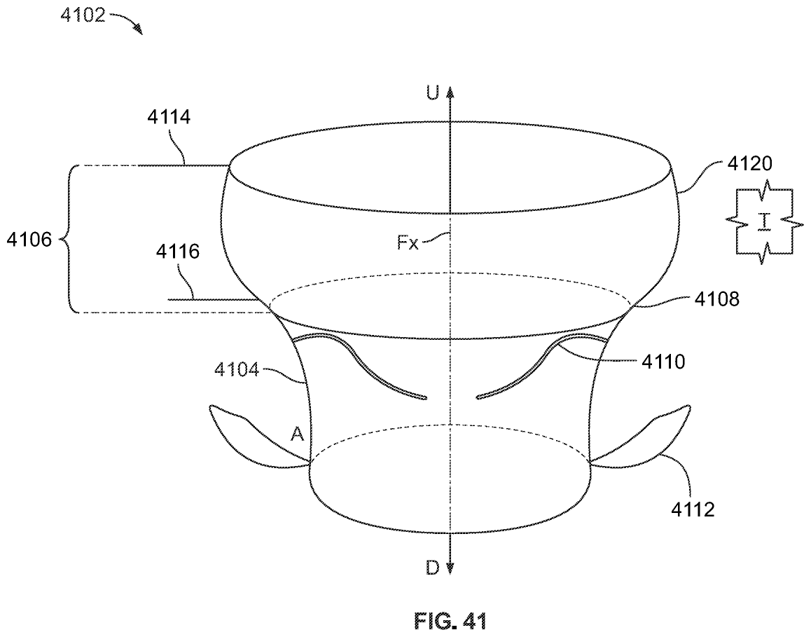

FIG. 41 shows illustrative prosthetic valve 4102 in valve annulus A. Valve 4102 may include fitting 4104. Fitting 4104 may support valve 4102 in a tricuspid annulus. Valve 4102 may include one or more of the features that are shown or described in connection with FIGS. 2-14 and may have one or more features in common with one or more of the other valves shown or described herein. Fitting 4104 may push natural leaflets against A. Valve 4102 may include brace 4106. Brace 4106 may include open cup section 4116. Valve 4102 may define an inflection point 4108 between fitting 4104 and brace 4106. Inflection point 4108 may allow valve 4102 to be seated in annulus A. Valve 4102 may include anchor 4112. Anchor 4112 may anchor valve 4102 into the tricuspid annulus. Valve 4102 may include prosthetic leaflets 4110. Prosthetic leaflets may be attached to fitting 4104. Prosthetic leaflets may be attached to brace 4106.

Valve 4102 may include open cup 4116, with the upper portion of open cup 4116 facing in direction U upon implantation into the patient's right atrium. Open cup 4116 may define an edge 4114 in direction U. Valve 4102 may extend partially upward into the right atrium.

The open cup section 4116 may provide a radial anchoring force against the atrial tissue. The atrial tissue may be tissue T. The atrial tissue may be annulus A. The atrial tissue may be tissue between tissue T and annulus A. The section may funnel or curve smoothly down to a tube section also referred to fitting 4104 that descends in direction D through the annulus. The open cup section 4116 and fitting 4104 may define a fluid-flow pathway between the right atrium and right ventricle. At least one prosthetic leaflet 4110 may be attached within the fitting 4104 on an inner wall thereof. At least one prosthetic leaflet 4110 may be attached within the brace 4106 on an inner wall thereof. The prosthetic leaflets may be positioned at, or slightly downstream of, an inflection point 4108 between the open cup section and the cylindrical portion of the tube section also referred to as fitting 4104. On the outer wall of the tube section, near direction D end of fitting 4104, may include anchor 4112 which may extend away from fitting 4104 in a direction that extends outward from axis Fx, wherein the angle between anchor 4112 and fitting 4104 may be acute. The angle between anchor 4112 and axis Fx may be acute. Anchor 4112 may be provided to assist in anchoring the expanded device by engaging one or more of the right ventricle, the right atrium, the tricuspid annulus, and any other suitable anatomical feature.

In some cases, the D end of the tube section may curve inwardly in a symmetric inward curvature, or in an asymmetric curvature, wherein the curvature may be configured to aim or direct the outgoing blood flow to a specific location within the right ventricle. This may promote fluid flow efficiency. Anchoring elements may collapse against the outer wall of the tube section for transport and may further be biased to expand by rotating away from the outer wall to engage the right ventricle. The anchoring elements may be positioned to engage the right atrium. The anchoring elements may be positioned to engage the tricuspid annulus. Anchoring elements may include a single plane/flattened stent frame and/or may include a shape memory material such as Nitinol or the equivalent.

The prosthetic tricuspid valve device of FIG. 41 may include an expandable and collapsible stent in the open cup section and/or tube section to facilitate collapsed translation and delivery via a lumen in a delivery tube such as a delivery catheter or sheath. A fabric or tissue cover or skirt may be provided on the inner and/or outer surfaces of open cup and/or tube section of the device.

One or both tube section and the cup section, and any other suitable apparatus elements, may be assembled outside the patient and delivered in an assembled state. One or both tube section and the cup section, and any other suitable apparatus elements, may be delivered in an unassembled state. One or both tube section and the cup section, and any other suitable apparatus elements, may be assembled inside the patient. Deployment of the apparatus may thus be performed in one, two or more deliveries.

Valve 4102 may be configured for translation through a lumen of a delivery tube in a collapsed configuration and delivery to the subject heart chamber.

Brace 4106 may have an outer surface 4120. In operation, some or all the outer surface 4120 may be in contact with atrial tissue.

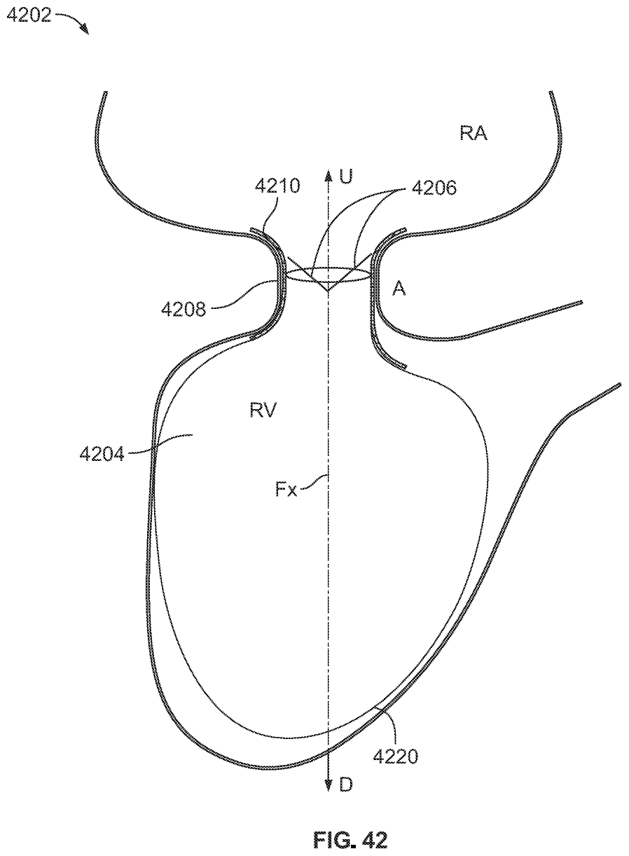

FIG. 42 shows illustrative prosthetic valve 4202 in valve annulus A. Valve 4202 may include one or more of the features that are shown or described in connection with FIGS. 2-14 and may have one or more features in common with one or more of the other valves shown or described herein. Valve 4202 may include fitting 4210. Fitting 4210 may support valve 4202 in a tricuspid annulus. Fitting 4210 may push natural tricuspid leaflets 4208 against annulus A. Valve 4202 may include brace 4204. Brace 4204 may be a ball frame. Valve 4202 may include leaflet 4206. Leaflet 4206 may be in the tricuspid annulus. Leaflet 4206 may be attached to fitting 4210.

Valve 4202 may be implanted at the level and below the level of the native tricuspid. Valve 4202 may be implanted above the level, at the level, and below the level of the native tricuspid.

Valve 4202 may include an expandable and collapsible frame defining an outer section that may have an inner surface and may have an outer surface. Brace 4204 may expand to anchor within right ventricle RV. Brace 4204 may be referred to as ball frame.

Fitting 4210 may also be referred to as a valve support. Fitting 4210 may be provided and may be operatively engaged with natural tricuspid leaflets 4208. Natural tricuspid leaflets 4208 include natural tricuspid leaflet. Fitting may be supported by the outer section. Fitting 4210 may include one or more of: inflow end, which may extend in direction U away from the outer section into the annulus, outflow end, inner surface, and outer surface.

Brace 4204 may be formed by an outward turn in the collapsible and expandable stent cell and an inward turn occurring across a transition section, operatively disposed to transition the device between the outer section and the inner valve support. The transition section may enable the brace 4204 to be inverted within the interior of the outer section. Thus, the inflow end of the valve support may be located radially inside the interior of the outer section. A stent device made of two or more pieces are coupled together. Brace 4204 may define a flow channel between the inflow and outflow ends and, respectively, along the inner surface of brace 4204. Prosthetic valve leaflets 4206 may be positioned at any location along the inner surface of fitting 4210. Native tricuspid leaflets 4208 may be pinned against the annular tissue by fitting 4210. Fitting 4210 may be a valve support that does not extend in direction U a distance that is sufficient to pin the native leaflets. This may leave the tricuspid native leaflet function intact. The native leaflet function may be supplemented by the prosthetic leaflets and, as the native leaflets continue to degrade in functionality, may eventually replace the native leaflet function.

Prosthetic leaflets 4206 may function as a one-way valve, allowing flow through flow channel F in the outflow, direction D and may reduce or prevent regurgitating flow in direction U. At least a portion of the outer surface of fitting 4210 may be covered by a fabric or tissue covering. The fabric or tissue may aid in apposition. The fabric or tissue may reduce or prevent paravalvular leakage. Portions of the inner surface of fitting 4210 may be covered by a fabric or tissue covering. The fabric or tissue may aid in fluid flow. The fabric or tissue may prevent or reduce thrombosis. Some or all of an inner surface of valve 4202 may be covered with a fabric or tissue covering. Some or all of a transition section of valve 4202 may be covered with a fabric or tissue covering. Some or all or an outer surface of valve 4202 may be covered with a fabric or tissue covering.

Valve 4204 may include paddles. The paddles may be used to anchor within the right atrium RA. A collapsible and expandable flange, or partial flange, may extend away in a radial direction from the upper surface of fitting 4204 to provide right atrium anchoring. The upper portion of valve 4202 may engage the wall tissue of the right ventricle RV. The right atrium RA paddles or flange may be used to provide anchoring. The paddles or flange may be flexible. The paddles or flange may be biased to fold against the outer surface fitting 4210 so that the flange may be in a deformed configuration providing downward force against the RA tissue as flange seeks to achieve the undeformed configuration.

One or more of the brace 4204, fitting 4210, leaflets 4206, and any other suitable apparatus elements, may be assembled outside the patient and delivered in an assembled state. One or more of the brace 4204, fitting 4210, leaflets 4206, and any other suitable apparatus elements, may be delivered in an unassembled state. One or more of the brace 4204, fitting 4210, leaflets 4206, and any other suitable apparatus elements, may be assembled inside the patient. Deployment of the apparatus may thus be performed in one, two, three or more deliveries.

A prosthetic tricuspid valve device 4204 for expanded implantation into RV of a patient's heart and treating native tricuspid valve leaflet regurgitation may include an expandable and collapsible ball-shaped brace 4204 comprising stent cells, a fitting 4210 formed from the stent cells and turned radially outwardly away from the expandable and collapsible ball-shaped brace 4204 at an upstream end of the anchor section, and one or more leaflets 4206 operatively attached to an inner side of fitting 4210.

Valve 4202 may be configured for translation through a lumen of a delivery tube in a collapsed configuration and delivery to the subject heart chamber.

Brace 4206 may have an outer surface 4220. In operation, some or all the outer surface 4220 may be in contact with atrial tissue.

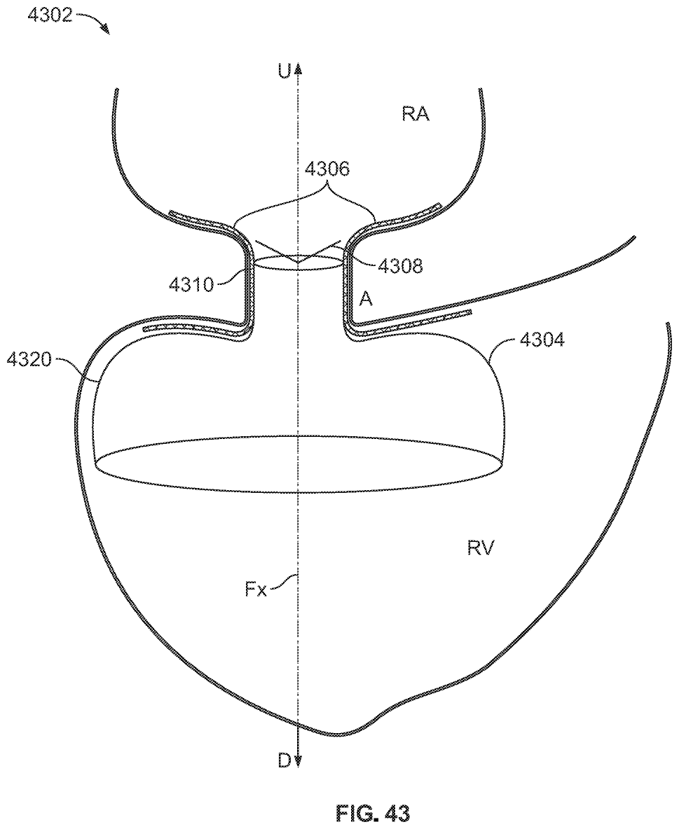

FIG. 43 shows illustrative prosthetic valve 4302 in valve annulus A. Valve 4302 may include one or more of the features that are shown or described in connection with FIGS. 2-14 and may have one or more features in common with one or more of the other valves shown or described herein. Valve 4302 may include fitting 4310. Fitting 4310 may support valve 4302 in a tricuspid annulus. Fitting 4310 may push native leaflets against annulus A. Valve 4302 may include brace 4304. Brace 4304 may be an open cup frame. Brace 4304 may be in RV. Valve 4302 may include a RA flange 4306. RA flange 4306 may assist in anchoring valve 4302 in annulus A. Brace 4304 may assist in anchoring valve 4302 in annulus A. Valve 4302 may include prosthetic leaflets 4308. Leaflets 4308 may be attached to fitting 4310. Leaflets 4308 may be attached to RA flange 4306.

Valve 4302 may include brace 4304 which may be an open cup frame. The open cup frame may include a stent that does not form a complete enclosure. The open cup frame may provide an opening in direction D. Valve 4302 may have similarities to valve 4102 in FIG. 41, but is inverted, so that fitting 4310 may extend in direction U away from the expandable and collapsible open cup frame. The open cup frame may be expanded in RV. Valve 4302 may include paddles. Such paddles may be used to anchor, within the right atrium, valve 4302. A collapsible and expandable flange 4306, or partial flange, may extend away in a radial direction from the upper surface of the valve support as shown to provide RA anchoring. The upper portion of the open cup frame may engage the top surface and portions of the side wall tissue of the RV. The RA paddles or flange may be used to provide anchoring. The paddles or flange may be flexible. The paddles or flange 4306 may be biased to fold against the outer surface of fitting 4310 so that the flange 4306 may be in a deformed configuration providing downward force against the RA tissue as flange 4306 seeks to achieve the undeformed configuration.

A prosthetic tricuspid valve device 4302 for expanded implantation into RV of a patient's heart and treating native tricuspid valve leaflet regurgitation may include an expandable and collapsible brace 4304 comprising stent cells, where brace 4304 is open at a downstream end, fitting 4310 formed from the stent cells and turned radially outwardly from the expandable and collapsible frame at direction U of the frame, and one or more prosthetic leaflets 4308 attached to an inner side of fitting 4310. Valve 4302 may include a right atrial supplemental anchoring elements 4306 of the group consisting of one or more paddles, and one or more flanges.

Valve 4302 may be configured for translation through a lumen of a delivery tube in a collapsed configuration and delivery to the subject heart chamber.

Brace 4306 may have an outer surface 4320. In operation, some or all the outer surface 4320 may be in contact with atrial tissue.

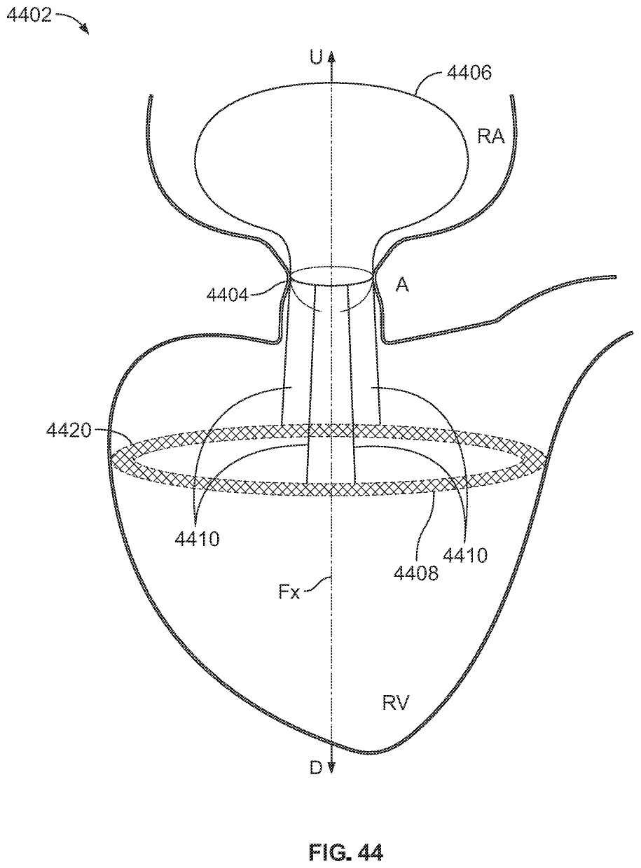

FIG. 44 shows illustrative prosthetic valve 4402 in valve annulus A. Valve 4402 may include one or more of the features that are shown or described in connection with FIGS. 2-14 and may have one or more features in common with one or more of the other valves shown or described herein. Valve 4402 may include fitting 4404. Fitting 4404 may support valve 4402 in a tricuspid annulus. Fitting 4404 may push natural leaflets against annulus A. Valve 4402 may include a brace. The brace may include an element in the atrium and an element in the ventricle. Atrium brace 4406 and ventricle brace 4408 may assist in keeping fitting 4404 in annulus A. Ventricle brace 4408 may include a RV anchor. Anchor strut 4410 may connect fitting 4404 to ventricle brace 4408. Anchor strut 4410 may be attached at its direction U end to fitting 4404. Anchor strut 4410 may be attached at its direction U end to fitting 4404. Anchor strut 4410 may extend in direction D to ventricle brace 4408. Ventricle brace 4408 may be expanded against the RV tissue. Ventricle brace 4408 may include a circumferential expandable and collapsible stent structure.

Valve 4402 may include prosthetic leaflets. Prosthetic leaflets may be attached to fitting 4404. Prosthetic leaflets may be attached to atrial brace 4406. Prosthetic leaflets may be attached to anchor struts 4410.

Ventricle brace 4408 may have an outer surface 4420. In operation, some or all the outer surface 4420 may be in contact with RV tissue.

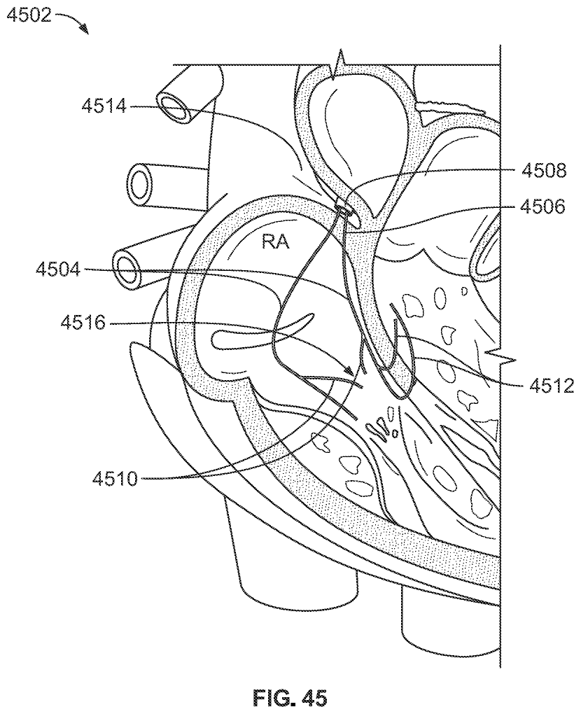

FIG. 45 shows illustrative prosthetic valve 4502 in valve annulus A. Valve 4502 may include one or more of the features that are shown or described in connection with FIGS. 2-14 and may have one or more features in common with one or more of the other valves shown or described herein. Valve 4502 may include prosthetic leaflet support wires 4504. Leaflet support wires 4504 may support prosthetic leaflets 4510 in or near a tricuspid annulus 4516. Leaflet support wires 4504 extend, in a direction away from the leaflets, through a septal access hole 4506 in the septic wall 4514 and are capped on the other side of the septic wall by an anchor cap 4508. At least one leaflet support wire 4504 may anchor into the septal wall in a downstream direction near the tricuspid annulus. This anchor 4512 may be shaped like a shield.

Valve 4502 may be anchored in the septal wall between the right and left atria. Anchor cap 4508 may be provided on the left atrial side of the septal wall, access may be provided through a puncture hole in the septal access hole 4506. The anchor cap 4508 may have a diameter that is larger than the puncture hole diameter to ensure anchoring. The anchor cap 4508 may have one or more barbs to engage septal wall tissue, the barbs may contain bioabsorbable or resorbable material. The anchor cap 4508 may include a flange. The flange may be expandable. The flange may be deployed on a distal side of the puncture hole. The flange may be expanded to engage tissue surrounding the puncture hole. The anchor cap may include a second flange. The second flange may be extendable. The second flange may be deployed on a proximal side of the puncture hole. The flange may be expanded to engage tissue surrounding the puncture hole. The two flanges may be drawn together to compress the tissue surrounding the puncture hole.

A plurality of prosthetic leaflet support wires 4504 may be attached at a proximal end to the anchor cap and extend through the puncture hole in the septal access hole 4506. The prosthetic leaflet support wires 4504 may contain a distal end and one or more prosthetic leaflets attached or connected to each of the prosthetic leaflet support wires 4504. As shown, the prosthetic leaflet(s) 4510 may be attached proximate to, and proximal of, the distal end of each of the prosthetic leaflet support wires 4504. The prosthetic leaflets 4510 may be attached at the distal end of the relevant prosthetic leaflet support wire 4504.

The prosthetic leaflet support wires 4504 may be curved in a middle portion. That curvature may be used to create a radially outward biasing force by the prosthetic leaflet support wire 4504 against the annulus to help anchor the wires and prosthetic leaflets in the correct position. Thus, in some cases the curvature may contain a deformation that creates the outwardly biased radial force when the distal end of the wire is placed within the annulus.

A native leaflet shield 4512 may be provided at or near the distal ends of the prosthetic leaflet support wires 4504 and may be distal to the attachment points of the prosthetic leaflet(s) 4510 to the prosthetic leaflet support wires 4504. The shield 4512 may be at the same location as the prosthetic leaflet attachment points.

If present, this shield structure 4512 may provide a biasing force that is directed radially outward against the annular tissue. This may help stabilize and support valve 4502 and prosthetic leaflets 4510. The shield 4512 may wrap all the way around the subject annulus or may wrap part of the way around. The shield 4512 may contain a single wire or may contain more than one spaced apart wire as shown in FIG. 45. A layer of stent cells may contain the shield. The shield 4512 may pin the native leaflets against the annular tissue. The shield may be a shield that does not pin the native leaflets against the annular tissue. The native leaflets may be positioned to extend through gaps in shield wires or cells so that the native leaflets may retain any functionality and the prosthetic leaflets provide a supplementary function, then a replacement function as the native leaflets begin to fail.

One deployment method may involve creating the puncture hole through the septal access hole 4506, then installing the anchor cap 4508. The remaining components may then be deployed, with attachment or connection to the anchor cap 4508.

One or more of the components, and any other suitable apparatus elements, may be assembled outside the patient and delivered in an assembled state. One or more of the components, and any other suitable apparatus elements, may be delivered in an unassembled state. One or more of the components, and any other suitable apparatus elements, may be assembled inside the patient. Deployment of the apparatus may thus be performed in one, two, three or more deliveries.

The device may be introduced as a unit. When introduced as a unit, the wires are passed through the puncture hole in the septal wall and the anchor cap, connected with the wires, is installed at the septal wall hole, with subsequent deployment and positioning of the wires and related prosthetic leaflets.

The device may be configured such that one or more of the prosthetic leaflets replaces one or more of the tricuspid valve leaflets. The device may be configured such that one or more of the prosthetic leaflets supplements one or more of the tricuspid valve leaflets.

The device may be configured such that one or more of the prosthetic leaflets replaces one or more of the mitral valve leaflets. The device may be configured such that one or more of the prosthetic leaflets supplements one or more of the mitral valve leaflets.

Valve 4502 for treating a patient with tricuspid valve regurgitation may include a septal anchor cap 4508 configured to anchor the device a plurality of prosthetic leaflet support wires 4504 comprising proximal and distal ends. The prosthetic leaflet support wires 4504 may be attached to the septal anchor cap 4508 at the proximal end, and one or more prosthetic leaflets 4510 attached proximate to the distal end of each prosthetic leaflet support wire 4504, where each prosthetic leaflet support wire 4504 may include a curvilinear middle portion between the proximal and distal ends. Valve 4502 may contain a curvilinear middle portion that comprises a deformed configuration and wherein the distal end of each prosthetic leaflet support wire seeks to return to an undeformed configuration. Valve 4502 may contain one or more prosthetic leaflets 4510 which may be disposed at the end of each prosthetic leaflet support wire. Valve 4502 may contain shield structure 4512 operationally connected proximate the distal end of the prosthetic leaflet support wires 4504.

All ranges and parameters disclosed herein shall be understood to encompass any and all subranges subsumed therein, every number between the endpoints, and the endpoints. For example, a stated range of “1 to 10” should be considered to include any and all subranges between (and inclusive of) the minimum value of 1 and the maximum value of 10; that is, all subranges beginning with a minimum value of 1 or more (e.g. 1 to 6.1), and ending with a maximum value of 10 or less (e.g., 2.3 to 10.4, 3 to 8, 4 to 7), and finally to each number 1, 2, 3, 4, 5, 6, 7, 8, 10, and 10 contained within the range.

Thus, apparatus and methods for a prosthetic heart valve have been provided. Persons skilled in the art will appreciate that the present invention can be practiced by other than the described examples, which are presented for purposes of illustration rather than of limitation. The present invention is limited only by the claims that follow.

1-238. (canceled)

239. Apparatus for treating a heart, the apparatus comprising:
a prosthetic heart-valve fitting that is configured to be seated in a valve annulus of the heart;
a brace that:
is configured to be urged by an atrial tissue of the heart to maintain a position of the fitting in the annulus; and
includes:
a first arm; and
a second arm that is disposed less than 180°, about a central axis of the fitting, away from the first arm.

240. The apparatus of claim 239 wherein:
the fitting has an outside diameter;
the first arm extends to the outside diameter; and
the second arm extends to the outside diameter.

241. The apparatus of claim 240 wherein the first arm and the second arm intersect each other at a central axis defined by the fitting.

242. The apparatus of claim 239 further comprising a circumferential stabilizer that extends circumferentially, relative to a central axis defined by the fitting, from the first arm to the second arm.

243. The apparatus of claim 242 wherein the circumferential stabilizer is disposed, in a direction of the central axis, in a plane that is perpendicular to the central axis.

244. The apparatus of claim 242 wherein circumferential stabilizers and arms extend circumferentially for 360°, in a direction of the central axis, in a plane that is perpendicular to the central axis.

245. The apparatus of claim 239 wherein, when the heart is in a delivery state and the apparatus is deployed in the heart:
the brace provides clearance along an access path that extends from an entrance of a chamber to an exit of the chamber; and
the clearance is not less than that required for passage of a 30 French instrument.

246-248. (canceled)

249. The apparatus of claim 245 wherein the entrance is a superior vena cava and the exit is a septal wall or the inferior vena cava.

250-252. (canceled)

253. The apparatus of claim 239 defining a window that:
accommodates a node of a cardiac conduction system; and,
in operation, faces the node;
wherein the node is a sinoatrial node or an atrioventricular node.

254. (canceled)

255. The apparatus of claim 253 wherein the window is large enough to accommodate the node and a margin around the node.

256. The apparatus of claim 255 wherein the margin has an area that is 2% to 10% larger than an area of the node.

257. The apparatus of claim 253 wherein the brace defines the window.

258-259. (canceled)

260. The apparatus of claim 239 further comprising a stand that extends from the fitting;
wherein:
the stand defines a surface opposite where the stand extends from the fitting;
the first arm extends to the surface; and
the second arm extends to the surface.

261. The apparatus of claim 260 wherein, when the heart is in a delivery state and the apparatus is deployed in the heart:
the brace provides clearance along an access path that extends from an entrance of a chamber to an exit of the chamber; and
the clearance is not less than that required for passage of a 30 French instrument.

262-268. (canceled)

269. The apparatus of claim 260 defining a window that:
accommodates a node of a cardiac conduction system; and,
in operation, faces the node;
wherein the node is a sinoatrial node or an atrioventricular node.

270-341. (canceled)

342. The apparatus of claim 239 including mesh that comprises cells;
wherein:
the mesh contains a first cell that defines a window; and
the first cell has a size that is larger than second cells in the mesh.

343. The apparatus of claim 240, wherein the brace has a first end and a second end, and further comprises:
a span that extends from the first end to the second end;
wherein:
the first end is connected to the fitting; and
the second end contacts the atrial tissue.

344. The apparatus of claim 343, wherein the span has a width that, when the apparatus is in an unconstrained state, is a maximum of 0.8× the outside diameter.

345. The apparatus of claim 260 wherein:
the first arm and the second arm extend away from the stand in a direction that is parallel to the central axis and then curves toward the central axis;
the first arm extends from a first position on the stand to a second position on the stand;
the second arm extends from a third position on the stand; and
the first arm and the second arm intersect each other.

346. The apparatus of claim 245 wherein the entrance is an inferior vena cava, and the exit is a septal wall.
